(19) United States
(12) Patent Application Publication
(10) Pub. No.: US 2017/0351806 A1
(43) Pub. Date: Dec. 7, 2017

(54) METHOD FOR ASSESSING FERTILITY BASED ON MALE AND FEMALE GENETIC AND PHENOTYPIC DATA

(71) Applicant: Celmatix Inc., New York, NY (US)

(72) Inventor: Piraye Yurttas Beim, New York, NY (US)

(21) Appl. No.: 15/610,058

(22) Filed: May 31, 2017

Related U.S. Application Data
(60) Provisional application No. 62/381,916, filed on Aug. 31, 2016.
(60) Provisional application No. 62/345,526, filed on Jun. 3, 2016.

(51) Int. Cl. G06F 19/18; C12Q 1/68

(52) U.S. Cl. CPC G06F 19/18; C12Q 1/6874

(57) ABSTRACT

The present invention generally relates to systems and methods for assessing female fertility and infertility, male fertility and infertility and the combined fertility profile of a male and a female. Systems and methods of the invention determine the fertility potential of a female and a male combined by conducting an assay on a sample obtained from the male and female to determine the presence of one or more fertility-associated genetic variants, obtain fertility-associated phenotypic and/or environmental data from the male and the female, accepting as input data, the genetic variants determined from the female and male and phenotypic and/or environmental exposure data from the male and female, analyze the input data using a prognosis predictor correlated with fertility, and generate a fertility profile that reflects the fertility potential of the male and the female combined by using the prognosis predictor on the input data.

### CROSS-REFERENCE TO RELATED APPLICATIONS

This application claims priority to and the benefit of U.S. Provisional Patent Application Ser. No. 62/345,526, filed Jun. 3, 2016, and U.S. Provisional Patent Application Ser. No. 62/381,916, filed Aug. 31, 2016, the contents of each of which are incorporated by reference herein in their entirety.

### TECHNICAL FIELD

The invention generally relates to methods for assessing the combined fertility profile of a male and a female.

### BACKGROUND

Approximately one in seven couples has difficulty conceiving. Infertility may be due to a single cause in either or both partner(s), or a combination of factors (e.g., genetic factors, diseases, or environmental factors) that may prevent a pregnancy from occurring or continuing. With respect to female infertility, every woman will become infertile in her lifetime due to menopause. On average, egg quality and number begins to decline precipitously at 35. However, some women experience this decline much earlier in life, while a number of women are fertile well into their 40's. Though, generally, advanced maternal age (35 and above) is associated with poorer fertility outcomes, there is at current time no way of diagnosing egg quality issues in younger women or knowing when a particular woman will start to experience decline in her egg quality or reserve, such that fertility is impacted.

In addition to female infertility, it is estimated that for around a third of couples unable to conceive a child, subfertility of the male partner is the sole explanation. This subfertility remains unexplained for almost half of these men, even after extensive clinical evaluation.

From the time a couple seeks medical assistance for difficulty conceiving, the couple is advised to undergo a number of diagnostic procedures to ascertain potential causes for why the couple is having difficulty conceiving. Often the procedures can be highly invasive, costly, and time consuming. Thus, there is a need for faster, non-invasive methods of assessing infertility. Additionally, given that couples are attempting to conceive well into their 30s and 40s, it may also be desirable for the couple to assess their fertility prior to any attempts to conceive.

### SUMMARY

The invention provides methods for assessing fertility and or infertility in by taking into consideration one or more factors, such as genetic variations (e.g., mutations, polymorphisms, expression levels) and phenotypic traits or environmental exposures in order to arrive at an assessment of fertility. According to the invention, certain genetic polymorphisms give rise to a predisposition to conditions that affect fertility, such as primary ovarian insufficiency or premature decline in ovarian function in a woman, which reduces egg count and/or viability, or for example, reduced sperm motility in a man. Moreover, specific combinations of genetic polymorphisms are significant with respect to a couple's combined fertility status.

As discussed below, an array of genetic information concerning the status of, for example, various fertility-associated genes, such as maternal effect genes, is used in order to assess fertility status. The genetic information may include one or more polymorphisms in one or more infertility-related genetic regions, mutations in one or more of those genetic regions, or particular epigenetic signatures affecting the expression of those genetic regions. The molecular consequence of variants in one or more of those regions could be one or a combination of the following: alternative splicing, lowered or increased RNA expression, and/or alterations in protein expression. These alterations could also include a different protein product being produced, such as one with reduced or increased activity, or a protein that elicits an abnormal immunological reaction. All of this information is significant in terms of informing a couple of their fertility profile.

In addition to looking exclusively at genomic information, the invention also contemplates combining genetic information (e.g., polymorphisms, mutations, etc.) with phenotypic and/or environmental data, methods of the invention to provide an additional level of clinical clarity. For example, polymorphisms in genes discussed below may provide information about a couple's fertility. However, in certain cases, the clinical outcome may not be determinative unless combined with certain phenotypic and/or environmental information. Thus, methods of the invention provide for a combination of genetic predispositional analysis in combination with phenotypic and environmental exposure data in order to assess the couple's fertility potential.

Certain aspects of the invention provide methods for assessing infertility in a couple that involve conducting an assay on at least a portion of an infertility-related genetic region in the female and the male to determine presence or absence of one or more variants in a plurality of genes in which the presence of a variant in at least one of those genes is indicative of infertility. Variants detected according to the invention may be any type of genetic variant. Exemplary variants include a single nucleotide polymorphism, a deletion, an insertion, an inversion, other rearrangements, a copy number variation, chromosomal microdeletion, genetic mosaicism, karyotype abnormality, or a combination thereof, as shown in FIG. 2. Any method of detecting genetic variants is useful with methods of the invention, and numerous methods are known in the art. In certain embodiments, sequencing is used to determine the presence of genetic variants. In particularly-preferred embodiments, the sequencing is sequencing-by-synthesis.

In other embodiments, one or more assays are performed on a gene product. In particular embodiments, the gene product is a product of a fertility-associated gene. The gene product may be RNA or protein. Any assay known in the art may be used to analyze the gene product(s). In certain embodiments, the assay involves determining an amount of the gene product and comparing the determined amount to a reference.

Methods of the invention may further involve obtaining a sample from the mammal that includes the plurality of infertility-related genes. The sample may be a human tissue or body fluid. In particular embodiments, samples are derived from both the male and female partners who are trying to conceive. The sample may be collected at any age before, during, or after puberty. In particular embodiments, the sample from the female is of maternal origin, such as blood or saliva, and the sample from the male is from semen. Methods of the invention may also involve enriching the sample for the plurality of fertility-related genes.

Methods of the invention are applicable to female fertility and infertility, male fertility and infertility, or combined male and female fertility and infertility. Examples of application to male fertility and infertility are shown below in Example 14.

In certain embodiments, an infertility-associated phenotypic trait or environmental exposure is used in combination with genomic results in order to assess fertility. Exemplary “phenotypic traits” include age, cholesterol levels, body mass index, and combinations thereof. Exemplary “environmental exposures” include smoking, alcohol intake, diet, residence history, or combinations thereof.

In one aspect of the invention, a method of determining the fertility potential of a female and a male combined is provided, including the steps of conducting an assay on a sample obtained from the female to determine the presence of one or more fertility-associated genetic biomarkers; conducting an assay on a sample obtained from the male to determine the presence of one or more fertility-associated genetic biomarkers; obtaining fertility-associated phenotypic and/or environmental data from the male and the female; accepting as input data, the genetic biomarkers determined from the female and male and phenotypic and/or environmental exposure data from the male and female; analyzing the input data using a prognosis predictor correlated with fertility and generated by obtaining training data from a reference set of females and males, wherein the training data corresponds to fertility-associated characteristics including male and female fertility-associated genetic biomarkers and fertility-associated phenotypic and environmental data, determining one or more correlations between the data and a known pregnancy outcome, training the prognosis predictor with said training data to provide outputs indicative of fertility; and generating a fertility profile that reflects the fertility potential of the male and the female combined by using the prognosis predictor on the input data.

In another aspect of the invention, a method of determining the fertility potential of a female and a male combined is provided that includes the steps of conducting an assay on a sample obtained from the female to determine the presence of one or more genetic variants associated with fertility; conducting an assay on a sample obtained from the male to determine the presence of one or more genetic variants associated with fertility; obtaining infertility-associated phenotypic characteristics and/or environmental exposure data from the male and the female; accepting as input data, the genetic variants determined from the female and male and phenotypic and/or environmental data from the male and female; identifying variables predictive of infertility from genetic, infertility-associated phenotypic and environmental exposure data obtained from a reference set of males and females;

generating weighted predictor variables based on a magnitude of change in fertility attributed to each predictor variable; and applying the weighted predictor variables to the input data to generate a fertility profile that reflects the fertility potential of the male and the female combined.

In addition to providing information to couples related to their fertility profile or risk of infertility, methods of the invention may also be used by a physician for treatment purposes, e.g., allowing a physician to make vitamin/drug recommendations to help reduce or eliminate the risk to early-onset reduction in fertility. For example, data showing a variant in a gene that affects infertility may be used by a physician to generate a treatment plan that may help remediate the infertility risk in a woman. For example, the physician may advise the woman to take a high dose of folic acid or other vitamin supplements/drugs in order to improve fertility.

### BRIEF DESCRIPTION OF THE DRAWINGS

FIG. 1 depicts the rate of decline of fertility with age and the corresponding increase in the risk of infertility with age in females.

FIG. 2 depicts the different kinds of genetic variants associated with risk of infertility.

FIG. 3 depicts important mammalian egg structures.

FIG. 4 depicts female reproduction/fertility related processes.

FIG. 5 depicts male reproduction/fertility related processes.

FIG. 6 depicts spermatogenic processes.

FIG. 7 depicts a method for filtering through variants detected in whole genome sequencing for the identification of genetic regions related to infertility.

FIG. 8 depicts some of the components of the Fertilome® Database, a tool for correlating genetic regions with risk for infertility (Fertilome® Score).

FIG. 9 is a bioinformatics pipeline used to identify biologically interesting and statistically significant genetic variants in infertile patients.

FIG. 10 depicts a methodology for integrating clinical data with genomic data to predict treatment dependent and independent fertility outcomes.

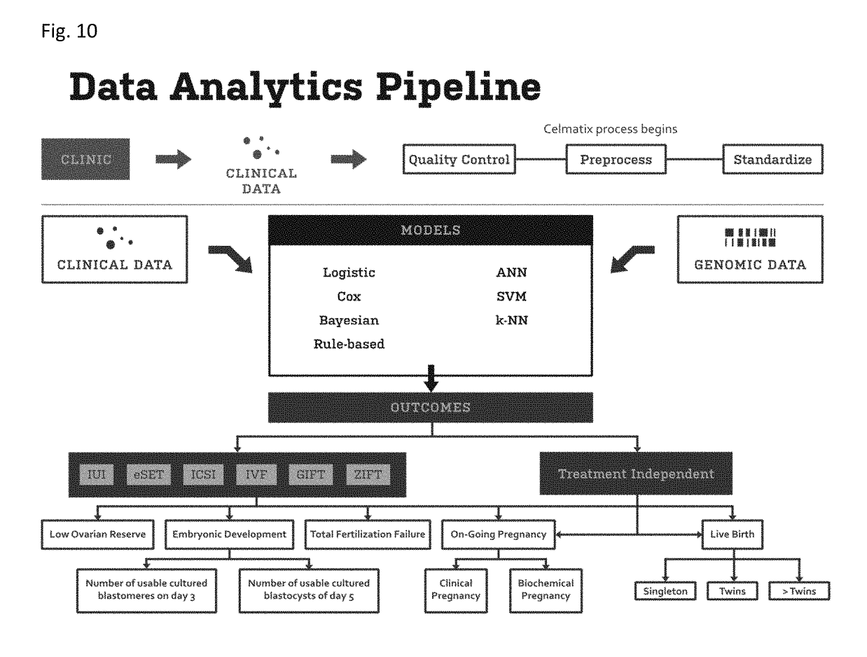

FIG. 11 illustrates population stratification correction of two patient groups (ZA=patients who did not get pregnant with IVF treatment, ZB=patients with infertility who did get pregnant with IVF treatment).

FIG. 12 depicts an area of the cluster analysis results.

FIG. 13 illustrates a system for implementing methods of the invention.

FIG. 14 depicts the procedural steps for determining the fertility profile of a couple, in accordance with one embodiment of the invention.

### DETAILED DESCRIPTION

Genetic variation, along with phenotypic and environmental factors, is used to assess infertility in a couple and can be used to select appropriate therapies and methods including in vitro fertilization. Methods of the invention analyze infertility-associated biomarkers and use results of that analysis to evaluate and/or quantify factors determinative of fertility in a couple, the couple being a man and a woman. For the purposes of this invention, use of the term “couple” also includes situations in which a sperm or egg donation and/or a surrogate is used to conceive a child, such that the donor and/or surrogate is one member of the “couple”.

Certain aspects of the invention are especially amenable for implementation using a computer. In those embodiments, systems and methods of the invention encompass a central processing unit (CPU) and storage coupled to the CPU. The storage stores instructions that when executed by the CPU, cause the CPU to accept as input data that is representative of a plurality of fertility-associated genotypic and phenotypic traits of a male and female subject. The executed instructions also cause the computer to provide a fertility profile. In one aspect, the profile can be generated as a result of comparing the input data to a reference set of data gathered from a plurality of men and women for whom fertility-associated characteristics are known.

The disclosed methods are also suitable when the female subject interested in having a child is not the one who will carry the baby. For example, if a surrogate is used, a couple may wish to know the likelihood that the surrogate can carry the embryo to live birth. Potential surrogates can include traditional and gestational surrogates. With a traditional surrogate, pregnancy may be achieved through insemination alone or through the assisted reproductive technologies described above, and the surrogate will be biologically related to the child. With a gestational carrier, eggs are removed from the female subject, fertilized with her partner's sperm, and transferred to the uterus of the gestational carrier. The gestational carrier will not be genetically related to the child. Whatever type of surrogate is used, the disclosed methods can also be applied to the surrogate as the primary (traditional) or secondary (gestational) female subject.

### Genotypic Data

It is known that certain genetic biomarkers are associated with infertility. Variations in these biomarkers may affect pregnancy outcomes. Therefore, in certain aspects of the invention, genotypic data is obtained from a couple.

Biomarkers, e.g., molecules that may act as an indicator of a biological state, for use with methods of the invention may be any marker that is associated with infertility. Exemplary biomarkers include genes (e.g. any region of DNA encoding a functional product), genetic regions (e.g. regions including genes and intergenic regions with a particular focus on regions conserved throughout evolution in placental mammals), and gene products (e.g., RNA and protein). In certain embodiments, the biomarker is an infertility-associated genetic region. An infertility-associated genetic region is any DNA sequence in which variation is associated with a change in fertility. Examples of changes in fertility include, but are not limited to, the following: a homozygous mutation of an infertility-associated gene leads to a complete loss of fertility; a homozygous mutation of an infertility-associated gene is incompletely penetrant and leads to reduction in fertility that varies from individual to individual; a heterozygous mutation is completely recessive, having no effect on fertility; and the infertility-associated gene is X-linked, such that a potential defect in fertility depends on whether a non-functional allele of the gene is located on an inactive X chromosome (Barr body) or on an expressed X chromosome.

In particular embodiments, the assessed infertility-associated genetic region is a maternal effect gene. Maternal effects genes are genes that have been found to encode key structures and functions in mammalian oocytes (Yurttas et al., Reproduction 139:809-823, 2010). Maternal effect genes are described, for example in, Christians et al. (Mol Cell Biol 17:778-88, 1997); Christians et al., Nature 407:693-694, 2000); Xiao et al. (EMBO J 18:5943-5952, 1999); Tong et al. (Endocrinology 145:1427-1434, 2004); Tong et al. (Nat Genet 26:267-268, 2000); Tong et al. (Endocrinology, 140:3720-3726, 1999); Tong et al. (Hum Reprod 17:903-911, 2002); Ohsugi et al. (Development 135:259-269, 2008); Borowczyk et al. (Proc Natl Acad Sci USA., 2009); and Wu (Hum Reprod 24:415-424, 2009). Maternal effects genes are also described in U.S. Ser. No. 12/889,304. The content of each of these is incorporated by reference herein in its entirety.

In particular embodiments, the infertility-associated genetic region is a gene (including exons, introns, and 10 kb of DNA flanking either side of said gene) selected from the genes shown in Table 1 below. In Table 1, OMIM reference numbers are provided when available.

TABLE 1
Human Infertility-Related Genes (OMIM #)

| | | | |
|---|---|---|---|
| ABCA1 (600046) | ACTL6A (604958) | ACTL8 | ACVR1 (102576) |
| ACVR1B (601300) | ACVR1C (608981) | ACVR2(102581) | ACVR2A (102581) |
| ACVR2B (602730) | ACVRL1 (601284) | ADA (608958) | ADAMTS1 (605174) |
| ADM (103275) | ADM2 (608682) | AFF2 (300806) | AGT (106150) |
| AHR (600253) | AIRE (607358) | AK2 (103020) | AK7 |
| AKR1C1 (600449) | AKR1C2 (600450) | AKR1C3 (603966) | AKR1C4 (600451) |
| AKT1 (164730) | ALDOA (103850) | ALDOB (612724) | ALDOC (103870) |
| ALPL (171760) | AMBP (176870) | AMD1 (180980) | AMH (600957) |
| AMHR2 (600956) | ANK3 (600465) | ANXA1 (151690) | APC (611731) |
| APOA1 (107680) | APOE (107741) | AQP4 (600308) | AR (313700) |
| AREG (104640) | ARF1 (103180) | ARF3 (103190) | ARF4 (601177) |
| ARF5 (103188) | ARFRP1 (604699) | ARL1 (603425) | ARL10 (612405) |
| ARL11 (609351) | ARL13A | ARL13B (608922) | ARL15 |
| ARL2 (601175) | ARL3 (604695) | ARL4A (604786) | ARL4C (604787) |
| ARL4D (600732) | ARL5A (608960) | ARL5B (608909) | ARL5C |
| ARL6 (608845) | ARL8A | ARL8B | ARMC2 |
| ARNTL (602550) | ASCL2 (601886) | ATF7IP (613644) | ATG7 (608760) |
| ATM (607585) | ATR (601215) | ATXN2 (601517) | AURKA (603072) |
| AURKB (604970) | AUTS2 (607270) | BARD1 (601593) | BAX (600040) |
| BBS1 (209901) | BBS10 (610148) | BBS12 (610683) | BBS2 (606151) |
| BBS4 (600374) | BBS5 (603650) | BBS7 (607590) | BBS9 (607968) |
| BCL2 (151430) | BCL2L1 (600039) | BCL2L10 (606910) | BDNF (113505) |
| BECN1 (604378) | BHMT (602888) | BLVRB (600941) | BMP15 (300247) |
| BMP2 (112261) | BMP3 (112263) | BMP4 (112262) | BMP5 (112265) |
| BMP6 (112266) | BMP7 (112267) | BMPR1A (601299) | BMPR1B (603248) |
| BMPR2 (600799) | BNC1 (601930) | BOP1 (610596) | BRCA1 (113705) |
| BRCA2 (600185) | BRIP1 (605882) | BRSK1 (609235) | BRWD1 |
| BSG (109480) | BTG4 (605673) | BUB1 (602452) | BUB1B (602860) |
| C2orf86 (613580) | C3 (120700) | C3orf56 | C6orf221 (611687) |
| CA1 (114800) | CARD8 (609051) | CARM1 (603934) | CASP1 (147678) |
| CASP2 (600639) | CASP5 (602665) | CASP6 (601532) | CASP8 (601763) |
| CBS (613381) | CBX1 (604511) | CBX2 (602770) | CBX5 (604478) |
| CCDC101 (613374) | CCDC28B (610162) | CCL13 (601391) | CCL14 (601392) |
| CCL4 (182284) | CCL5 (187011) | CCL8 (602283) | CCND1 (168461) |
| CCND2 (123833) | CCND3 (123834) | CCNH (601953) | CCS (603864) |
| CD19 (107265) | CD24 (600074) | CD55 (125240) | CD81 (186845) |
| CD9 (143030) | CDC42 (116952) | CDK4 (123829) | CDK6 (603368) |
| CDK7 (601955) | CDKN1B (600778) | CDKN1C (600856) | CDKN2A (600160) |
| CDX2 (600297) | CDX4 (300025) | CEACAM20 | CEBPA (116897) |
| CEBPB (189965) | CEBPD (116898) | CEBPE (600749) | CEBPG (138972) |
| CEBPZ (612828) | CELF1 (601074) | CELF4 (612679) | CENPB (117140) |
| CENPF (600236) | CENPI (300065) | CEP290 (610142) | CFC1 (605194) |
| CGA (118850) | CGB (118860) | CGB1 (608823) | CGB2 (608824) |
| CGB5 (608825) | CHD7 (608892) | CHST2 (603798) | CLDN3 (602910) |
| COIL (600272) | COL1A2 (120160) | COL4A3BP (604677) | COMT (116790) |
| COPE (606942) | COX2 (600262) | CP (117700) | CPEB1 (607342) |
| CRHR1 (122561) | CRYBB2 (123620) | CSF1 (120420) | CSF2 (138960) |
| CSTF1 (600369) | CSTF2 (600368) | CTCF (604167) | CTCFL (607022) |
| CTF2P | CTGF (121009) | CTH (607657) | CTNNB1 (116806) |
| CUL1 (603134) | CX3CL1 (601880) | CXCL10 (147310) | CXCL9 (601704) |
| CXorf67 | CYP11A1 (118485) | CYP11B1 (610613) | CYP11B2 (124080) |
| CYP17A1 (609300) | CYP19A1 (107910) | CYP1A1 (108330) | CYP27B1 (609506) |
| DAZ2 (400026) | DAZL (601486) | DCTPP1 | DDIT3 (126337) |
| DDX11 (601150) | DDX20 (606168) | DDX3X (300160) | DDX43 (606286) |
| DEPDC7 (612294) | DHFR (126060) | DHFRL1 | DIAPH2 (300108) |
| DICER1 (606241) | DKK1 (605189) | DLC1 (604258) | DLGAP5 |
| DMAP1 (605077) | DMC1 (602721) | DNAJB1 (604572) | DNMT1 (126375) |
| DNMT3B (602900) | DPPA3 (608408) | DPPA5 (611111) | DPYD (612779) |
| DTNBP1 (607145) | DYNLL1 (601562) | ECHS1 (602292) | EEF1A1 (130590) |
| EEF1A2 (602959) | EFNA1 (191164) | EFNA2 (602756) | EFNA3 (601381) |
| EFNA4 (601380) | EFNA5 (601535) | EFNB1 (300035) | EFNB2 (600527) |
| EFNB3 (602297) | EGR1 (128990) | EGR2 (129010) | EGR3 (602419) |
| EGR4 (128992) | EHMT1 (607001) | EHMT2 (604599) | EIF2B2 (606454) |
| EIF2B4 (606687) | EIF2B5 (603945) | EIF2C2 (606229) | EIF3C (603916) |
| EIF3CL (603916) | EPHA1 (179610) | EPHA10 (611123) | EPHA2 (176946) |
| EPHA3 (179611) | EPHA4 (602188) | EPHA5 (600004) | EPHA6 (600066) |
| EPHA7 (602190) | EPHA8 (176945) | EPHB1 (600600) | EPHB2 (600997) |
| EPHB3 (601839) | EPHB4 (600011) | EPHB6 (602757) | ERCC1 (126380) |
| ERCC2 (126340) | EREG (602061) | ESR1 (133430) | ESR2 (601663) |
| ESR2 (601663) | ESRRB (602167) | ETV5 (601600) | EZH2 (601573) |
| EZR (123900) | FANCC (613899) | FANCG (602956) | FANCL (608111) |
| FAR1 | FAR2 | FASLG (134638) | FBN1 (134797) |
| FBN2 (612570) | FBN3 (608529) | FBRS (608601) | FBRSL1 |
| FBXO10 (609092) | FBXO11 (607871) | FCRL3 (606510) | FDXR (103270) |
| FGF23 (605380) | FGF8 (600483) | FGFBP1 (607737) | FGFBP3 |
| FGFR1 (136350) | FHL2 (602633) | FIGLA (608697) | FILIP1L (612993) |
| FKBP4 (600611) | FMN2 (606373) | FMR1 (309550) | FOLR1 (136430) |
| FOLR2 (136425) | FOXE1 (602617) | FOXL2 (605597) | FOXN1 (600838) |
| FOXO3 (602681) | FOXP3 (300292) | FRZB (605083) | FSHB (136530) |
| FSHR (136435) | FST (136470) | GALT (606999) | GBPS (611467) |
| GCK (138079) | GDF1 (602880) | GDF3 (606522) | GDF9 (601918) |
| GGT1 (612346) | GJA1 (121014) | GJA10 (611924) | GJA3 (121015) |
| GJA4 (121012) | GJA5 (121013) | GJA8 (600897) | GJB1 (304040) |
| GJB2 (121011) | GJB3 (603324) | GJB4 (605425) | GJB6 (604418) |
| GJB7 (611921) | GJC1 (608655) | GJC2 (608803) | GJC3 (611925) |
| GJD2 (607058) | GJD3 (607425) | GJD4 (611922) | GNA13 (604406) |
| GNB2 (139390) | GNRH1 (152760) | GNRH2 (602352) | GNRHR (138850) |
| GPC3 (300037) | GPRC5A (604138) | GPRC5B (605948) | GREM2 (608832) |
| GRN (138945) | GSPT1 (139259) | GSTA1 (138359) | H19 (103280) |
| H1FOO (142709) | HABP2 (603924) | HADHA (600890) | HAND2 (602407) |
| HBA1 (141800) | HBA2 (141850) | HBB (141900) | HELLS (603946) |
| HK3 (142570) | HMOX1 (141250) | HNRNPK (600712) | HOXA11 (142958) |
| HPGD (601688) | HS6ST1 (604846) | HSD17B1 (109684) | HSD17B12 (609574) |
| HSD17B2 (109685) | HSD17B4 (601860) | HSD17B7 (606756) | HSD3B1 (109715) |
| HSF1 (140580) | HSF2BP (604554) | HSP90B1 (191175) | HSPG2 (142461) |
| HTATIP2 (605628) | ICAM1 (147840) | ICAM2 (146630) | ICAM3 (146631) |
| IDH1 (147700) | IFI30 (604664) | IFITM1 (604456) | IGF1 (147440) |
| IGF1R (147370) | IGF2 (147470) | IGF2BP1 (608288) | IGF2BP2 (608289) |
| IGF2BP3 (608259) | IGF2BP3 (608259) | IGF2R (147280) | IGFALS (601489) |
| IGFBP1 (146730) | IGFBP2 (146731) | IGFBP3 (146732) | IGFBP4 (146733) |
| IGFBP5 (146734) | IGFBP6 (146735) | IGFBP7 (602867) | IGFBPL1 (610413) |
| IL10 (124092) | IL11RA (600939) | IL12A (161560) | IL12B (161561) |
| IL13 (147683) | IL17A (603149) | IL17B (604627) | IL17C (604628) |
| IL17D (607587) | IL17F (606496) | IL1A (147760) | IL1B (147720) |
| IL23A (605580) | IL23R (607562) | IL4 (147780) | IL5 (147850) |
| IL5RA (147851) | IL6 (147620) | IL6ST (600694) | IL8 (146930) |
| ILK (602366) | INHA (147380) | INHBA (147290) | INHBB (147390) |
| IRF1 (147575) | ISG15 (147571) | ITGA11 (604789) | ITGA2 (192974) |
| ITGA3 (605025) | ITGA4 (192975) | ITGA7 (600536) | ITGA9 (603963) |
| ITGAV (193210) | ITGB1 (135630) | JAG1 (601920) | JAG2 (602570) |
| JARID2 (601594) | JMY (604279) | KAL1 (300836) | KDM1A (609132) |
| KDM1B (613081) | KDM3A (611512) | KDM4A (609764) | KDM5A (180202) |
| KDM5B (605393) | KHDC1 (611688) | KIAA0430 (614593) | KIF2C (604538) |
| KISS1 (603286) | KISS1R (604161) | KITLG (184745) | KL (604824) |
| KLF4 (602253) | KLF9 (602902) | KLHL7 (611119) | LAMC1 (150290) |
| LAMC2 (150292) | LAMP1 (153330) | LAMP2 (309060) | LAMP3 (605883) |
| LDB3 (605906) | LEP (164160) | LEPR (601007) | LFNG (602576) |
| LHB (152780) | LHCGR (152790) | LHX8 (604425) | LIF (159540) |
| LIFR (151443) | LIMS1 (602567) | LIMS2 (607908) | LIMS3 |
| LIMS3L | LIN28 (611043) | LIN28B (611044) | LMNA (150330) |
| LOC613037 | LOXL4 (607318) | LPP (600700) | LYRM1 (614709) |
| MAD1L1 (602686) | MAD2L1 (601467) | MAD2L1BP | MAF (177075) |
| MAP3K1 (600982) | MAP3K2 (609487) | MAPK1 (176948) | MAPK3 (601795) |
| MAPK8 (601158) | MAPK9 (602896) | MB21D1 (613973) | MBD1 (156535) |
| MBD2 (603547) | MBD3 (603573) | MBD4 (603574) | MCL1 (159552) |
| MCM8 (608187) | MDK (162096) | MDM2 (164785) | MDM4 (602704) |
| MECP2 (300005) | MED12 (300188) | MERTK (604705) | METTL3 (612472) |
| MGAT1 (160995) | MITF (156845) | MKKS (604896) | MKS1 (609883) |
| MLH1 (120436) | MLH3 (604395) | MOS (190060) | MPPED2 (600911) |
| MRS2 | MSH2 (609309) | MSH3 (600887) | MSH4 (602105) |
| MSH5 (603382) | MSH6 (600678) | MST1 (142408) | MSX1 (142983) |
| MSX2 (123101) | MTA2 (603947) | MTHFD1 (172460) | MTHFR (607093) |
| MTO1 (614667) | MTOR (601231) | MTRR (602568) | MUC4 (158372) |
| MVP (605088) | MX1 (147150) | MYC (190080) | NAB1 (600800) |
| NAB2 (602381) | NAT1 (108345) | NCAM1 (116930) | NCOA2 (601993) |
| NCOR1 (600849) | NCOR2 (600848) | NDP (300658) | NFE2L3 (604135) |
| NLRP1 (606636) | NLRP10 (609662) | NLRP11 (609664) | NLRP12 (609648) |
| NLRP13 (609660) | NLRP14 (609665) | NLRP2 (609364) | NLRP3 (606416) |
| NLRP4 (609645) | NLRP5 (609658) | NLRP6 (609650) | NLRP7 (609661) |
| NLRP8 (609659) | NLRP9 (609663) | NNMT (600008) | NOBOX (610934) |
| NODAL (601265) | NOG (602991) | NOS3 (163729) | NOTCH1 (190198) |
| NOTCH2 (600275) | NPM2 (608073) | NPR2 (108961) | NR2C2 (601426) |
| NR3C1 (138040) | NR5A1 (184757) | NR5A2 (604453) | NRIP1 (602490) |
| NRIP2 | NRIP3 (613125) | NTF4 (162662) | NTRK1 (191315) |
| NTRK2 (600456) | NUPR1 (614812) | OAS1 (164350) | OAT (613349) |
| OFD1 (300170) | OOEP (611689) | ORAI1 (610277) | OTC (300461) |
| PADI1 (607934) | PADI2 (607935) | PADI3 (606755) | PADI4 (605347) |
| PADI6 (610363) | PAEP (173310) | PAIP1 (605184) | PARP12 (612481) |
| PCNA (176740) | PCP4L1 | PDE3A (123805) | PDK1 (602524) |
| PGK1 (311800) | PGR (607311) | PGRMC1 (300435) | PGRMC2 (607735) |
| PIGA (311770) | PIM1 (164960) | PLA2G2A (172411) | PLA2G4C (603602) |
| PLA2G7 (601690) | PLAC1L | PLAG1 (603026) | PLAGL1 (603044) |
| PLCB1 (607120) | PMS1 (600258) | PMS2 (600259) | POF1B (300603) |
| POLG (174763) | POLR3A (614258) | POMZP3 (600587) | POU5F1 (164177) |
| PPID (601753) | PPP2CB (176916) | PRDM1 (603423) | PRDM9 (609760) |
| PRKCA (176960) | PRKCB (176970) | PRKCD (176977) | PRKCDBP |
| PRKCE (176975) | PRKCG (176980) | PRKCQ (600448) | PRKRA (603424) |
| PRLR (176761) | PRMT1 (602950) | PRMT10 (307150) | PRMT2 (601961) |
| PRMT3 (603190) | PRMT5 (604045) | PRMT6 (608274) | PRMT7 (610087) |
| PRMT8 (610086) | PROK1 (606233) | PROK2 (607002) | PROKR1 (607122) |
| PROKR2 (607123) | PSEN1 (104311) | PSEN2 (600759) | PTGDR (604687) |
| PTGER1 (176802) | PTGER2 (176804) | PTGER3 (176806) | PTGER4 (601586) |
| PTGES (605172) | PTGES2 (608152) | PTGES3 (607061) | PTGFR (600563) |
| PTGFRN (601204) | PTGS1 (176805) | PTGS2 (600262) | PTN (162095) |
| PTX3 (602492) | QDPR (612676) | RAD17 (603139) | RAX (601881) |
| RBP4 (180250) | RCOR1 (607675) | RCOR2 | RCOR3 |
| RDH11 (607849) | REC8 (608193) | REXO1 (609614) | REXO2 (607149) |
| RFPL4A (612601) | RGS2 (600861) | RGS3 (602189) | RSPO1 (609595) |
| RTEL1 (608833) | SAFB (602895) | SAR1A (607691) | SAR1B (607690) |
| SCARB1 (601040) | SDC3 (186357) | SELL (153240) | SEPHS1 (600902) |
| SEPHS2 (606218) | SERPINA10 (605271) | SFRP1 (604156) | SFRP2 (604157) |
| SFRP4 (606570) | SFRP5 (604158) | SGK1 (602958) | SGOL2 (612425) |
| SH2B1 (608937) | SH2B2 (605300) | SH2B3 (605093) | SIRT1 (604479) |
| SIRT2 (604480) | SIRT3 (604481) | SIRT4 (604482) | SIRT5 (604483) |
| SIRT6 (606211) | SIRT7 (606212) | SLC19A1 (600424) | SLC28A1 (606207) |
| SLC28A2 (606208) | SLC28A3 (608269) | SLC2A8 (605245) | SLC6A2 (163970) |
| SLC6A4 (182138) | SLCO2A1 (601460) | SLITRK4 (300562) | SMAD1 (601595) |
| SMAD2 (601366) | SMAD3 (603109) | SMAD4 (600993) | SMAD5 (603110) |
| SMAD6 (602931) | SMAD7 (602932) | SMAD9 (603295) | SMARCA4 (603254) |
| SMARCA5 (603375) | SMC1A (300040) | SMC1B (608685) | SMC3 (606062) |
| SMC4 (605575) | SMPD1 (607608) | SOCS1 (603597) | SOD1 (147450) |
| SOD2 (147460) | SOD3 (185490) | SOX17 (610928) | SOX3 (313430) |
| SPAG17 | SPARC (182120) | SPIN1 (609936) | SPN (182160) |
| SPO11 (605114) | SPP1 (166490) | SPSB2 (611658) | SPTB (182870) |
| SPTBN1 (182790) | SPTBN4 (606214) | SRCAP (611421) | SRD5A1 (184753) |
| SRSF4 (601940) | SRSF7 (600572) | ST5 (140750) | STAG3 (608489) |
| STAR (600617) | STARD10 | STARD13 (609866) | STARD3 (607048) |
| STARD3NL (611759) | STARD4 (607049) | STARD5 (607050) | STARD6 (607051) |
| STARD7 | STARD8 (300689) | STARD9 (614642) | STAT1 (600555) |
| STAT2 (600556) | STAT3 (102582) | STAT4 (600558) | STAT5A (601511) |
| STAT5B (604260) | STAT6 (601512) | STC1 (601185) | STIM1 (605921) |
| STK3 (605030) | SULT1E1 (600043) | SUZ12 (606245) | SYCE1 (611486) |
| SYCE2 (611487) | SYCP1 (602162) | SYCP2 (604105) | SYCP3 (604759) |
| SYNE1 (608441) | SYNE2 (608442) | TAC3 (162330) | TACC3 (605303) |
| TACR3 (162332) | TAF10 (600475) | TAF3 (606576) | TAF4 (601796) |
| TAF4B (601689) | TAF5 (601787) | TAF5L | TAF8 (609514) |
| TAF9 (600822) | TAP1 (170260) | TBL1X (300196) | TBXA2R (188070) |
| TCL1A (186960) | TCL1B (603769) | TCL6 (604412) | TCN2 (613441) |
| TDGF1 (187395) | TERC (602322) | TERF1 (600951) | TERT (187270) |
| TEX12 (605791) | TEX9 | TF (190000) | TFAP2C (601602) |
| TFPI (152310) | TFPI2 (600033) | TG (188450) | TGFB1 (190180) |
| TGFB1I1 (602353) | TGFBR3 (600742) | THOC5 (612733) | THSD7B |
| TLE6 (612399) | TM4SF1 (191155) | TMEM67 (609884) | TNF (191160) |
| TNFAIP6 (600410) | TNFSF13B (603969) | TOP2A (126430) | TOP2B (126431) |
| TP53 (191170) | TP53I3 (605171) | TP63 (603273) | TP73 (601990) |
| TPMT (187680) | TPRXL (611167) | TPT1 (600763) | TRIM32 (602290) |
| TSC2 (191092) | TSHB (188540) | TSIX (300181) | TTC8 (608132) |
| TUBB4Q (158900) | TUFM (602389) | TYMS (188350) | UBB (191339) |
| UBC (191340) | UBD (606050) | UBE2D3 (602963) | UBE3A (601623) |
| UBL4A (312070) | UBL4B (611127) | UIMC1 (609433) | UQCR11 (609711) |
| UQCRC2 (191329) | USP9X (300072) | VDR (601769) | VEGFA (192240) |
| VEGFB (601398) | VEGFC (601528) | VHL (608537) | VIM (193060) |
| VKORC1 (608547) | VKORC1L1 (608838) | WAS (300392) | WISP2 (603399) |
| WNT7A (601570) | WNT7B (601967) | WT1 (607102) | XDH (607633) |
| XIST (314670) | YBX1 (154030) | YBX2 (611447) | ZAR1 (607520) |
| ZFX (314980) | ZNF22 (194529) | ZNF267 (604752) | ZNF689 |
| ZNF720 | ZNF787 | ZNF84 | ZP1 (195000) |
| ZP2 (182888) | ZP3 (182889) | ZP4 (613514) | |

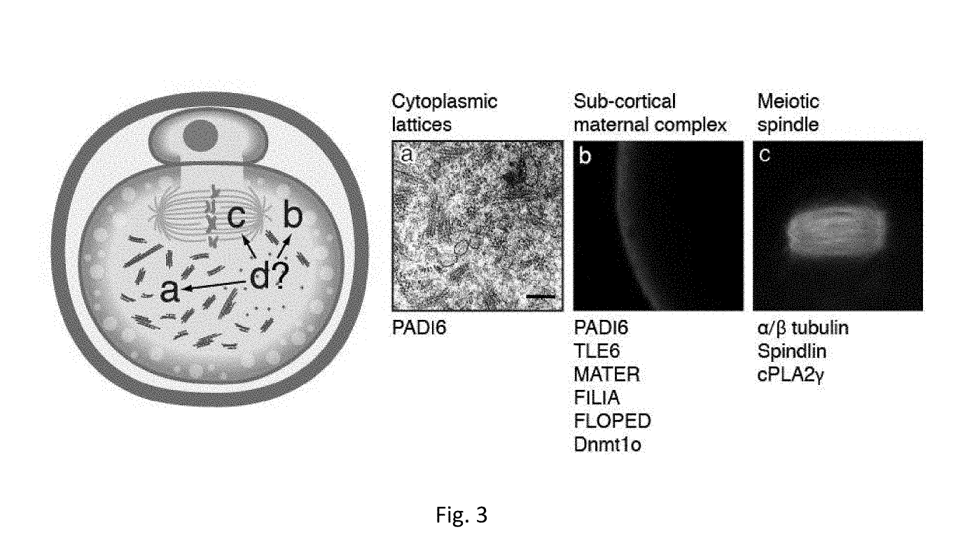

The molecular products of the genes in Table 1 are involved in different aspects of oocyte and embryo physiology from transcription and chromosome remodeling to RNA processing and binding. FIG. 3 depicts important mammalian egg structures: the Cytoplasmic Lattices, the Subcortical Maternal Complex (SCMC), and the Meiotic Spindle, that infertility-associated gene products localize to and regulate.

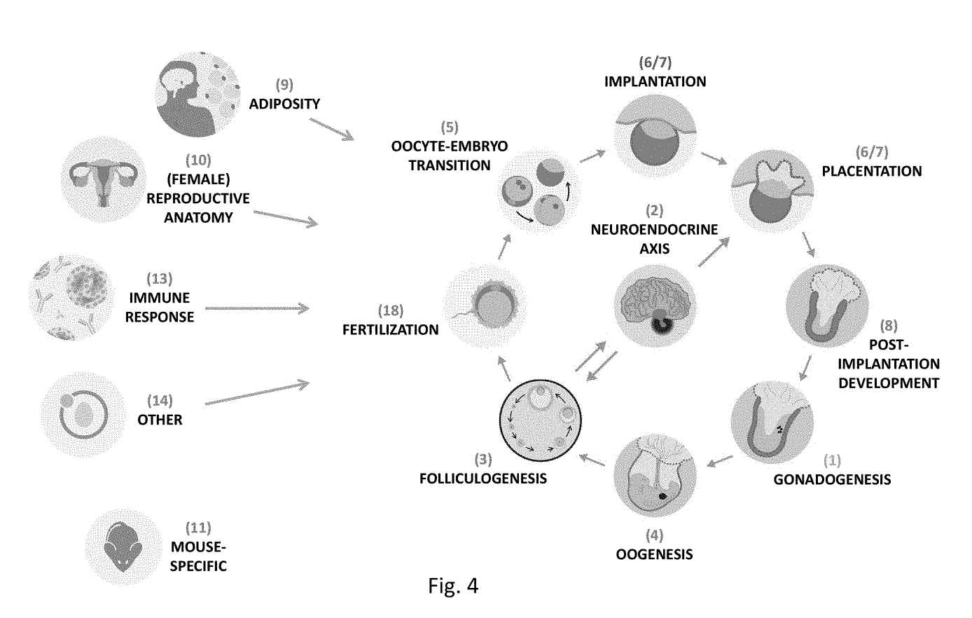

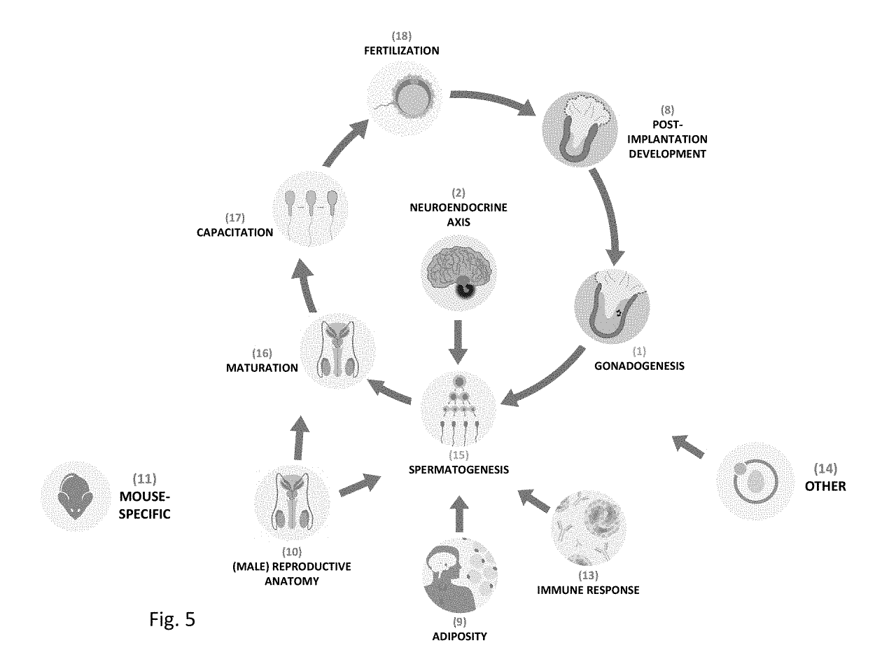

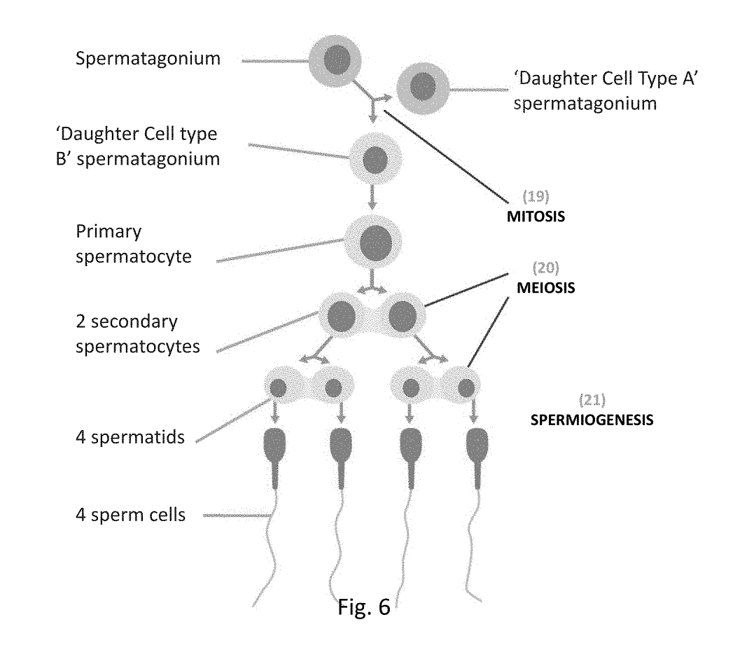

The genes listed in Table 1 can also be involved in different aspects of reproduction/fertility related processes. Furthermore additional genes beyond those maternal effect genes listed in Table 1 can also affect fertility. Genes affecting fertility can be involved with a number of male- and female-specific processes, such as those shown in FIGS. 4-6. As shown in FIG. 4, female reproductive/fertility related processes include gonadogenesis, neuroendocrine axis, folliculogensis, oogenesis, oocyte-embryo transition, placentation, post-implantation development, adiposity, (female) reproductive anatomy, immune response, fertilization and other processes. Male reproductive/fertility related processes include gonadogenesis neuroendocrine axis, post-implantation development, adiposity, (male) reproductive anatomy, immune response, spermatogenesis, sperm maturation and capacitation, fertilization, mitosis, meiosis, spermiogenesis, and other processes, as shown in FIGS. 5 and 6. These processes are described in more detail below.

Gonadogenesis encompasses the processes regulating the development of the ovaries and testes, and involves, but is not limited to, primordial germ cell specification and proliferation. The neuroendocrine axis encompasses for example the physiological pathways and structures regulating the production and activity of hormones in a number of different tissues in the human body, including the brain and gonads. Folliculogenesis encompasses the physiological mechanisms regulating the development of primordial follicles to cystic follicles in the ovary. Oogenesis encompasses the physiological mechanisms regulating the development of primordial oocytes to mature meiosis-II stage oocytes ready to be fertilized, hence those that are specific to female reproductive biology. Oocyte-embryo transition encompasses the physiological mechanisms regulating the development of the early embryo and includes mechanisms related to egg quality, such as oocyte cytoplasmic lattice formation, and paternal effect mechanisms. Placentation (Embryonic) encompasses the embryo-specific physiological mechanisms regulating implantation and the development of the placenta. Placentation (Uterine) encompasses the uterus-specific physiological mechanisms regulating embryo implantation and the development of the placenta. Post-implantation development encompasses the physiological mechanisms regulating post-implantation embryo development, particularly those whose disruption might lead to abnormal development or pregnancy loss in humans. Adiposity encompasses the physiological mechanisms regulating adipose tissue and body weight, which are known to play an important, indirect role in mammalian fecundity and infertility. Reproductive anatomy encompasses any phenotype relating to anatomical changes that could impact reproduction, fecundity or fertility. Immune response encompasses phenotypes that are specific to aspects of immune response mechanisms, which are known to play an important role in mammalian reproduction and fertility.

Spermatogenesis encompasses the processes involved in the production or development of mature spermatozoa, hence those that are specific to male reproductive biology. Maturation encompasses processes that enable spermatozoa to fertilize eggs, hence those that are specific to male reproductive biology. Capacitation encompasses processes specific to functional capacitation of spermatozoa in the vaginal canal and uterus. Fertilization encompasses processes relating to the union of a human egg and sperm. Mitosis encompasses processes involving changes to the cell division process such that it does not end with two daughter cells that have the same chromosomal complement as the parent cell. Such changes to the mitotic process may affect for example fertility-related cell proliferation or tissue maintenance. Meiosis encompasses processes regulating meiosis such that it results in four daughter cells each with exactly half the chromosome complement of the parent cell, for example during gametogenesis. Spermiogenesis encompasses processes regulating the morphological differentiation of haploid cells into sperm.

Variants in genes associated with these various processes result in fertility difficulties for males and/or females containing these variants. Exemplary genes that affect fertility are further described below.

BRCA1-Associated Ring Domain 1 (BARD1) encodes a protein that forms a heterodimer complex with the BRCA1 gene product, and this complex is required for spindle-pole assembly in mitosis, and hence chromosome stability. Mouse embryos carrying homozygous null alleles for BARD1 died between embryonic day 7.5 and embryonic day 8.5 due to severely impaired cell proliferation (McCarthy et al. Molec. Cell. Biol. 23: 5056-5063, 2003).

KH domain containing 3-like, subcortical maternal complex member (KHDC3L). The gene also has the identifier “C6orf221” [Entrez Gene id: 154288, HGNC id: 33699] and is a human homologue of the Khdc3l/FILIA mouse gene. FILIA was identified and named for its interaction with MATER (Ohsugi et al. Development 135:259-269, 2008). KH domains are protein domains that binds to RNA molecules, and KHDC3L is likely involved in genomic imprinting, a phenomenon where genes are expressed in a parental-origin specific manner. KHDC3L gene expression is maximal in germinal vesicle oocytes, tailing off through metaphase II oocytes, and its expression profile is similar to other oocyte-specific genes [Am J Hum Genet. 2011 Sep. 9; 89(3): 451-458]. It is also found within the set of maternal factors constituting the subcortical maternal complex (SCMC), which are important for driving the egg-to-embryo transition during fertilization [Reproduction. 2010 May; 139(5):809-23]. Like other components of the SCMC, maternal inheritance of the Khdc3/KHDC3L gene product is required for early embryonic development. In humans, KHDC3L has been implicated in familial biparental hydatidiform mole, a maternal-effect recessive inherited disorder (Am J Hum Genet. 2011 Sep. 9; 89(3): 451-458). Loss of Khdc3 in mice results in aneuploidy, due to spindle checkpoint assembly (SAC) inactivation, abnormal spindle assembly, and chromosome misalignment (Zheng et al. Proc Natl Acad Sci USA 106:7473-7478, 2009). Thus, mice carrying homozygous null alleles for Khdc3 display a maternal effect defect in embryogenesis with delayed embryonic development and decreased litter sizes for homozygous females (Li et al., 2008).

DNA (cytosine-5)-methyltransferase 1 (DNMT1) [Entrez Gene id: 1786, HGNC id: 2976], belongs to a group of enzymes that transfer methyl groups to position 5 of cytosine bases in DNA. While this process, known as DNA methylation, does not alter DNA base composition, it leaves “epigenetic” modifications to DNA molecules that affect the biochemical properties of the DNA region. DNA methylation, mediated by DNMT1, is crucial in determining cell fate during embryogenesis (Genes Dev. 2008 Jun. 15; 22(12):1607-16, Dev Biol. 2002 Jan. 1; 241(1):172-82). Mouse embryos carrying homozygous null alleles for DNMT1 survive only to mid-gestation. The expression of the DNMT1 gene is significantly higher in reproductive tissues than other cell types, and is found within the set of maternal factors that are important for driving egg-to-embryo transition during fertilization (Reproduction. 2010 May; 139(5):809-23, BMC Genomics. 2009 Aug. 3; 10:348)].

Factor in Germline Alpha (FIGLA) [Entrez Gene id: 344018, HGNC id: 24669], also goes by the gene identifiers POF6, BHLHC8, and FIGALPHA. This gene product is a basic helix-loop-helix transcription factor that acts as an activator of oocyte genes. FIGLA is expressed in all ovarian follicular stages and in mature oocytes, and is required for normal folliculogenesis. FIGLA expression is also believed to repress genes expressed normal in male testes, and hence sustains the female phenotype by activating female and repressing male germ cell genetic hierarchies in growing oocytes during postnatal ovarian development (Mol Cell Biol. 2010 July; 30(14)). Female mice with FIGLA mutations result in decreased oocytes numbers and abnormal ovarian folliculogenesis. Heterozygous mutations in FIGLA has been implicated in women with premature ovarian failure (Am J Hum Genet. 2008 June; 82(6):1342-8).

Fragile X Mental Retardation 1 (FMR1) encodes for the RNA-binding protein FMRP that is implicated in the fragile-X syndrome. The inhibition of translation may be a function of FMR1 in vivo, and that failure of mutant FMR1 protein to oligomerize may contribute to the pathophysiologic events leading to fragile X syndrome. Fragile X premutations in female carriers appear to be a risk factor for premature ovarian failure: 16% of the premutation carriers, menopause occurred before the age of 40, compared with none of the full-mutation carriers and 1 (0.4%) of the controls, indicating a significant association between premature menopause and premutation carrier status. (Am. J. Med. Genet. 83: 322-325, 1999).

Forkhead box O3 (FOXO3) encodes a protein that induces apoptosis in cells, lying within the DNA damage response and repair pathways. FOXO3 knockout female mice exhibit infertility phenotypes, in particular abnormal ovarian follicular function. Mice mutants carrying a homozygous non-synonymous substitution in exon 2 of the FOXO3 gene show loss of fertility of sexual maturity and exhibit premature ovarian failures. (Mammalian Genome 22: 235-248, 2011).

Mucin 4 (MUC4) gene product belongs to a family of high-molecular-weight glycoproteins that protect and lubricate the epithelial surface of respiratory, gastrointestinal and reproductive tracts. The extracellular domain can interact with an epidermal growth factor receptor on the cell surface to modulate downstream cell growth signaling by stabilizing and/or enhancing the activity of cell growth receptor complexes (Nature Rev. Cancer. 4(1):45-60, 2004). MUC4 is expressed in the endometrial epithelium and is associated with endometriosis development and endometriosis-related infertility such as embryo implantation (BMC Med. 2011 9:19, 2011).

NLR family, pyrin domain containing 11 (NLRP11) encodes a leucine-rich protein belonging to a large family of proteins likely involved in inflammation (Nature Rev. Molec. Cell Biol. 4: 95-104, 2003), and is expressed in the ovary, testes and pre-implantation embryos (BMC Evol Biol. 2009 Aug. 14; 9:202. doi: 10.1186/1471-2148-9-202). NLRP11 gene expression shows specificity to reproductive tissues.

NLR family, pyrin domain containing 14 (NLRP14) encodes a leucine-rich protein belonging to a large family of proteins likely involved in inflammation [Nature Rev. Molec. Cell Biol. 4: 95-104, 2003], and is expressed in the ovary, testes and pre-implantation embryos [BMC Evol Biol. 2009 Aug. 14; 9:202. doi: 10.1186/1471-2148-9-202.]. NPRL14 is also found within the set of maternal factors that are important for driving egg-to-embryo transition during fertilization [Reproduction. 2010 May; 139(5):809-23, BMC Genomics. 2009 Aug. 3; 10:348].

NLR family, pyrin domain containing 8 (NLRP8) encodes a leucine-rich protein belonging to a large family of proteins likely involved in inflammation [Nature Rev. Molec. Cell Biol. 4: 95-104, 2003], and is expressed in the ovary, testes and pre-implantation embryos [BMC Evol Biol. 2009 Aug. 14; 9:202. doi: 10.1186/1471-2148-9-202.]. NLRP8 gene expression shows specificity to reproductive tissues.

Postmeiotic Segregation Increased 2 (PMS2) is involved in DNA mismatch repair and involved in fertilization and pre-implantation development. It has been identified by knockout mouse studies as one of many maternal effect genes essential for development [Nature Cell Bio. 4 Suppl, pp. s41-9].

Scavenger receptor class B, member 1 (SCARB1) gene encodes a glycoprotein that is a receptor for mediating cholesterol transport. SCARB1-null homozygous female mice were infertile with dysfunctional oocytes [J. Clin. Invest. 108: 1717-1722, 2001], hence, mutations in SCARB1 may affect female fertility by regulating lipoprotein metabolism.

Spindlin 1 (SPIN1) is a gene abundantly expressed in early embryo development, during the transition from oocyte to pluripotent early-embryo. SPIN1 is phosphorylated in a cell-cycle dependent manner and is associated with the meiotic spindle [Development 124: 493-503, 1997].

Zona pellucida glycoprotein 1 (ZP1) encodes for a protein that is a structural component of the zona pellucida—an extracellular matrix that surrounds the oocyte and early embryo.

Zona pellucida glycoprotein 2 (ZP2) encodes for a protein that is a structural component of the zona pellucida—an extracellular matrix that surrounds the oocyte and early embryo. ZP2 binds to acrosome-reacted sperm and is important in preventing polyspermy [Hum Reprod. 2004 July; 19(7):1580-6.].

Zona pellucida glycoprotein 3 (ZP3) [Entrez Gene id: 7784, HGNC id: 13189], is a structural component of the zona pellucida—an extracellular matrix that surrounds the oocyte and early embryo. It is found within the set of maternal factors that are important for driving egg-to-embryo transition during fertilization [BMC Genomics. 2009 Aug. 3; 10:348]. ZP3 is also expressed in oocytes from early ovarian development, and likely to have a role in the development of primordial follicle before zona pellucida formation [Mol Cell Endocrinol. 2008 Jul. 16; 289(1-2):10-5]. Female mice carrying null alleles for ZP3 exhibit decreased ovary size and weight, abnormal ovarian folliculogenesis and ovulation, ultimately resulting in female infertility.

Zona pellucida glycoprotein 4 (ZP4) encodes for a protein that is a structural component of the zona pellucida—an extracellular matrix that surrounds the oocyte and early embryo. ZP4 stimulates acrosome reaction as part of a signaling pathway that involves Protein Kinase A [Biol Reprod. 2008 November; 79(5):869-77]

Peptidylarginine deiminase 6 (PADI6) Padi6 was originally cloned from a 2D murine egg proteome gel based on its relative abundance, and Padi6 expression in mice appears to be almost entirely limited to the oocyte and pre-implantation embryo (Yurttas et al., 2010). Padi6 is first expressed in primordial oocyte follicles and persists, at the protein level, throughout pre-implantation development to the blastocyst stage (Wright et al., Dev Biol. 256:73-88, 2003). Inactivation of Padi6 leads to female infertility in mice, with the Padi6-null developmental arrest occurring at the two-cell stage (Yurttas et al., 2008).

Nucleoplasmin 2 (NPM2) Nucleoplasmin is another maternal effect gene, and is thought to be phosphorylated during mouse oocyte maturation. NPM2 exhibits a phosphate sensitive increase in mass during oocyte maturation. Increased phosphorylation is retained through the pronuclear stage of development. NPM2 then becomes dephosphorylated at the two-cell stage and remains in this form throughout the rest of pre-implantation development. Further, its expression pattern appears to be restricted to oocytes and early embryos. Immunofluorescence analysis of NPM2 localization shows that NPM2 primarily localizes to the nucleus in mouse oocytes and early embryos. In mice, maternally-derived NPM2 is required for female fertility (Burns et al., 2003).

Maternal antigen the embryos require (MATER/NLRP5) MATER is another highly abundant mouse oocyte protein that is essential for embryonic development beyond the two-cell stage. MATER was originally identified as an oocyte-specific antigen in a mouse model of autoimmune premature ovarian failure (Tong et al., Endocrinology, 140:3720-3726, 1999). MATER demonstrates a similar expression and subcellular expression profile to PADI6. Like PADI6 null animals, MATER null females exhibit normal oogenesis, ovarian development, oocyte maturation, ovulation and fertilization. However, embryos derived from Mater-null females undergo a developmental block at the two-cell stage and fail to exhibit normal embryonic genome activation (Tong et al., Nat Genet 26:267-268, 2000; and Tong et al. Mamm. Genome 11:281-287, 2000b).

SWI/SNF related, matrix associated, actin dependent regulator of chromatin, subfamily a, member 4 (SMARCA4, aka BRG1). Mammalian SWI/SNF-related chromatin remodeling complexes regulate transcription and are believed to be involved in zygotic genome activation (ZGA). Such complexes are composed of approximately nine subunits, which can be variable depending on cell type and tissue. The BRG1 catalytic subunit exhibits DNA-dependent ATPase activity, and the energy derived from ATP hydrolysis alters the conformation and position of nucleosomes. Brg1 is expressed in oocytes and has been shown to be essential in the mouse as null homozygotes do not progress beyond the blastocyst stage (Bultman et al., 2000).

Oocyte expressed protein (OOEP, aka FLOPED). The subcortical maternal complex (SCMC) is a poorly characterized murine oocyte structure to which several maternal effect gene products localize (Li et al. Dev Cell 15:416-425, 2008). PADI6, MATER, FILIA, TLE6, and FLOPED have been shown to localize to this complex (Li et al. Dev Cell 15:416-425, 2008; Yurttas et al. Development 135:2627-2636, 2008). This complex is not present in the absence of Floped and Nlrp5, and similar to embryos resulting from Nlrp5-depleted oocytes, embryos resulting from Floped-null oocytes do not progress past the two cell stage of mouse development (Li et al., 2008). FLOPED is a small (19 kD) RNA binding protein that has also been characterized under the name of MOEP19 (Herr et al., Dev Biol 314:300-316, 2008).

Basonuclin (BNC1) Basonuclin is a zinc finger transcription factor that has been studied in mice. It is found expressed in keratinocytes and germ cells (male and female) and regulates rRNA (via polymerase I) and mRNA (via polymerase II) synthesis (Iuchi and Green, 1999; Wang et al., 2006). Depending on the amount by which expression is reduced in oocytes, embryos may not develop beyond the 8-cell stage. In Bsn1 depleted mice, a normal number of oocytes are ovulated even though oocyte development is perturbed, but many of these oocytes cannot go on to yield viable offspring (Ma et al., 2006).

Zygote Arrest 1 (ZAR1) Zar1 is an oocyte-specific maternal effect gene that is known to function at the oocyte to embryo transition in mice. High levels of Zar1 expression are observed in the cytoplasm of murine oocytes, and homozygous-null females are infertile: growing oocytes from Zar1-null females do not progress past the two-cell stage.

Phospholipase A2 group IV C (PLA2G4C, aka cPLA2γ). Under normal conditions, cPLA2γ expression is restricted to oocytes and early embryos in mice. At the subcellular level, cPLA2γ mainly localizes to the cortical regions, nucleoplasm, and multivesicular aggregates of oocytes. It is also worth noting that while cPLA2γ expression does appear to be mainly limited to oocytes and pre-implantation embryos in healthy mice, expression is considerably up-regulated within the intestinal epithelium of mice infected with Trichinella spiralis. This suggests that cPLA2γ may also play a role in the inflammatory response. The human cPLA2γ orthologue differs in that rather than being abundantly expressed in the ovary, it is abundantly expressed in the heart and skeletal muscle. Also, the human protein contains a lipase consensus sequence but lacks a calcium binding domain found in other PLA2 enzymes. Accordingly, another cytosolic phospholipase may be a better candidate.

Transforming, Acidic Coiled-Coil Containing Protein 3 (TACC3) In mice, Maskin/TACC3 is required for microtubule anchoring at the centrosome and for spindle assembly and cell survival.

In certain embodiments, the gene is a gene that is expressed in an oocyte. Exemplary genes include CTCF, ZFP57, POU5F1, SEBOX, and HDAC1. In other embodiments, the gene is a gene that is involved in DNA repair pathways, including but not limited to, MLH1, PMS1 and PMS2. In other embodiments, the gene is BRCA1 or BRCA2.

In other embodiments, the biomarker is a gene product (e.g., RNA or protein) of an infertility-associated gene. In particular embodiments, the gene product is a gene product of a maternal effect gene. In other embodiments, the gene product is a product of a gene from Table 1. In certain embodiments, the gene product is a product of a gene that is expressed in an oocyte, such as a product of CTCF, ZFP57, POU5F1, SEBOX, and HDAC1. In other embodiments, the gene product is a product of a gene that is involved in DNA repair pathways, such as a product of MLH1, PMS1, or PMS2. In other embodiments, the gene product is a product of BRCA1 or BRCA2.

In other embodiments, the biomarker may be an epigenetic factor, such as methylation patterns (e.g., hypermethylation of CpG islands), genomic localization or post-translational modification of histone proteins, or general post-translational modification of proteins such as acetylation, ubiquitination, phosphorylation, or others.

In certain embodiments, the biomarker is a genetic region, gene, or RNA/protein product of a gene associated with the one carbon metabolism pathway and other pathways that effect methylation of cellular macromolecules. Exemplary genes and products of those genes are described below.

Methylenetetrahydrofolate Reductase (MTHFR) In particular embodiments a mutation (677C>T) in the MTHFR gene is associated with infertility. The enzyme 5,10-methylenetetrahydrofolate reductase regulates folate activity (Pavlik et al., Fertility and Sterility 95(7): 2257-2262, 2011). The 677TT genotype is known in the art to be associated with 60% reduced enzyme activity, inefficient folate metabolism, decreased blood folate, elevated plasma homocysteine levels, and reduced methylation capacity. Pavlik et al. (2011) investigated the effect of the MTHFR 677C>T on serum anti-Mullerian hormone (AMH) concentrations and on the numbers of oocytes retrieved (NOR) following controlled ovarian hyperstimulation (COH). Two hundred and seventy women undergoing COH for IVF were analyzed, and their AMH levels were determined from blood samples collected after 10 days of GnRH superagonist treatment and before COH. Average AMH levels of TT carriers were significantly higher than those of homozygous CC or heterozygous CT individuals. AMH serum concentrations correlated significantly with the NOR in all individuals studied. The study concluded that the MTHFR 677TT genotype is associated with higher serum AMH concentrations but paradoxically has a negative effect on NOR after COH. It was proposed that follicle maturation might be retarded in MTHFR 677TT individuals, which could subsequently lead to a higher proportion of initially recruited follicles that produce AMH, but fail to progress towards cyclic recruitment. The tissue gene expression patterns of MTHFR do not show any bias towards oocyte expression. Analyzing a sample for this mutation or other mutations (Table 1) in the MTHFR gene or abnormal gene expression of products of the MTHFR gene allows one to assess a risk of infertility.

Jeddi-Tehrani et al. (American Journal of Reproductive Immunology 66(2):149-156, 2011) investigated the effect of the MTHFR 677TT genotype on Recurrent Pregnancy Loss (RPL). One hundred women below 35 years of age with two successive pregnancy losses and one hundred healthy women with at least two normal pregnancies were used to assess the frequency of five candidate genetic risk factors for RPL—MTHFR 677C>T, MTHFR 1298A>C, PAI1 −675 4G/5G (Plasminogen Activator Inhibitor-1 promoter region), BF −455G/A (Beta Fibrinogen promoter region), and ITGB3 1565T/C (Integrin Beta 3). The frequencies of the polymorphisms were calculated and compared between case and control groups. Both the MTHFR polymorphisms (677C>T and 1298 A>C) and the BF −455G/A polymorphism were found to be positively and ITGB3 1565T/C polymorphism was found to be negatively associated with RPL. Homozygosity but not heterozygosity for the PAI-1 −6754G/5G polymorphism was significantly higher in patients with RPL than in the control group. The presence of both mutations of MTHFR genes highly increased the risk of RPL. Analyzing a sample for these mutation and other mutations (Table 1) in the MTHFR gene or abnormal gene expression of products of the MTHFR gene allows one to assess a risk of infertility.

Catechol-O-methyltransferase (COMT) In particular embodiments a mutation (472G>A) in the COMT gene is associated with infertility. Catechol-O-methyltransferase is known in the art to be one of several enzymes that inactivates catecholamine neurotransmitters by transferring a methyl group from SAM (S-adenosyl methionine) to the catecholamine. The AA gene variant is known to alter the enzyme's thermostability and reduces its activity 3 to 4 fold (Schmidt et al., Epidemiology 22(4): 476-485, 2011). Salih et al. (Fertility and Sterility 89(5, Supplement 1): 1414-1421, 2008) investigated the regulation of COMT expression in granulosa cells and assessed the effects of 2-ME2 (COMT product) and COMT inhibitors on DNA proliferation and steroidogenesis in JC410 porcine and HGL5 human granulosa cell lines in in vitro experiments. They further assessed the regulation of COMT expression by DHT (Dihydrotestosterone), insulin, and ATRA (all-trans retinoic acid). They concluded that COMT expression in granulosa cells was up-regulated by insulin, DHT, and ATRA. Further, 2-ME2 decreased, and COMT inhibition increased granulosa cell proliferation and steroidogenesis. It was hypothesized that COMT overexpression with subsequent increased level of 2-ME2 may lead to ovulatory dysfunction. Analyzing a sample for this mutation in the COMT gene or abnormal gene expression of products of the COMT gene allows one to assess a risk of infertility.

Methionine Synthase Reductase (MTRR) In particular embodiments a mutation (A66G) in the Methionine Synthase Reductase (MTRR) gene is associated with infertility. MTRR is required for the proper function of the enzyme Methionine Synthase (MTR). MTR converts homocysteine to methionine, and MTRR activates MTR, thereby regulating levels of homocysteine and methionine. The maternal variant A66G has been associated with early developmental disorders such as Down's syndrome (Pozzi et al., 2009) and Spina Bifida (Doolin et al., American journal of human genetics 71(5): 1222-1226, 2002). Analyzing a sample for this mutation in the MTRR gene or abnormal gene expression of products of the MTRR gene allows one to assess the risk of infertility.

Betaine-Homocysteine S-Methyltransferase (BHMT) In particular embodiments a mutation (G716A) in the BHMT gene is associated with infertility. Betaine-Homocysteine S-Methyltransferase (BHMT), along with MTRR, assists in the Folate/B-12 dependent and choline/betaine-dependent conversions of homocysteine to methionine. High homocysteine levels have been linked to female infertility (Berker et al., Human Reproduction 24(9): 2293-2302, 2009). Benkhalifa et al. (2010) discuss that controlled ovarian hyperstimulation (COH) affects homocysteine concentration in follicular fluid. Using germinal vesicle oocytes from patients involved in IVF procedures, the study concludes that the human oocyte is able to regulate its homocysteine level via remethylation using MTR and BHMT, but not CBS (Cystathione Beta Synthase). They further emphasize that this may regulate the risk of imprinting problems during IVF procedures. Analyzing a sample for this mutation in the BHMT gene or abnormal gene expression of products of the BHMT gene allows one to assess a risk of infertility.

Ikeda et al. (Journal of Experimental Zoology Part A: Ecological Genetics and Physiology 313A(3): 129-136, 2010) examined the expression patterns of all methylation pathway enzymes in bovine oocytes and preimplantation embryos. Bovine oocytes were demonstrated to have the mRNA of MAT1A (Methionine adenosyltransferase), MAT2A, MAT2B, AHCY (S-adenosylhomocysteine hydrolase), MTR, BHMT, SHMT1 (Serine hydroxymethyltransferase), SHMT2, and MTHFR. All these transcripts were consistently expressed through all the developmental stages, except MAT1A, which was not detected from the 8-cell stage onward, and BHMT, which was not detected in the 8-cell stage. Furthermore, the effect of exogenous homocysteine on preimplantation development of bovine embryos was investigated in vitro. High concentrations of homocysteine induced hypermethylation of genomic DNA as well as developmental retardation in bovine embryos. Analyzing a sample for these irregular methylation patterns allows one to assess a risk of infertility.

Folate Receptor 2 (FOLR2) In particular embodiments a mutation (rs2298444) in the FOLR2 gene is associated with infertility. Folate Receptor 2 helps transport folate (and folate derivatives) into cells. Elnakat and Ratnam (Frontiers in bioscience: a journal and virtual library 11: 506-519, 2006) implicate FOLR2, along with FOLR1, in ovarian and endometrial cancers. Analyzing sample mutations in the FOLR2 or FOLR1 genes or abnormal gene expression of products of the FOLR2 or FOLR1 genes allows one to assess a risk of infertility.

Transcobalamin 2 (TCN2) In particular embodiments a mutation (C776G) in the TCN2 gene is associated with infertility. Transcobalamin 2 facilitates transport of cobalamin (Vitamin B12) into cells. Stanislawska-Sachadyn et al. (Eur J Clin Nutr 64(11): 1338-1343, 2010) assessed the relationship between TCN2 776C>G polymorphism and both serum B12 and total homocysteine (tHcy) levels. Genotypes from 613 men from Northern Ireland were used to show that the TCN2 776CC genotype was associated with lower serum B12 concentrations when compared to the 776CG and 776GG genotypes. Furthermore, vitamin B12 status was shown to influence the relationship between TCN2 776C>G genotype and tHcy concentrations. The TCN2 776C>G polymorphism may contribute to the risk of pathologies associated with low B12 and high total homocysteine phenotype. Analyzing a sample for this mutation in the TCN2 gene or abnormal gene expression of products of the TCN2 gene allows one to assess a risk of infertility.

Cystathionine-Beta-Synthase (CBS) In particular embodiments a mutation (rs234715) in the CBS gene is associated with infertility. With vitamin B6 as a cofactor, the Cystathionine-Beta-Synthase (CBS) enzyme catalyzes a reaction that permanently removes homocysteine from the methionine pathway by diverting it to the transsulfuration pathway. CBS gene mutations associated with decreased CBS activity also lead to elevated plasma homocysteine levels. Guzman et al. (2006) demonstrate that Cbs knockout mice are infertile. They further explain that Cbs-null female infertility is a consequence of uterine failure, which is a consequence of hyperhomocysteinemia or other factor(s) in the uterine environment. Analyzing a sample for this mutation in the CBS gene or abnormal gene expression of products of the CBS gene allows one to assess a risk of infertility.

Sirtuin 1 (SIRT1) A homolog of the yeast Sir2 protein, which regulates epigenetic gene silencing and suppresses recombination of rDNA histone. The catalytic domain regulating the deacetylase activity of Sirt1 is evolutionary conserved in the genomes of both primitive organisms and mammals (Frye 2000). Mice lacking the Sirt1 gene are not viable in inbred strain backgrounds and show pleiotropic phenotypes in outcrossed strains, including small size, developmental defects and sterility (McBurney et al., 2003). Mice that overexpress SIRT1 display lower levels of circulating free fatty acids, leptin and adiponectin (Bordone et al., 2007) and activation of SIRT1 by resveratrol has been observed to protect against age- and obesity-related infertility in mice (Liu et al., 2013, Zhou et al., 2014). In vitro experiments in human granulosa-like tumor cell lines suggest that SIRT1 is part of the positive feedback loop regulating estrogen synthesis in human granulosa cells (Zhang et al., 2016).

FK506 binding protein 4 (FKBP4, aka FKBP52). Member of the immunophilin protein family, which play a role in immunoregulation and basic cellular processes involving protein folding and trafficking. FKBP4 is an isomerase that binds to the immunosuppressants FK506 and rapamycin. FKBP4 is expressed in both male and female reproductive organs, including testis, ovary and uterus (Cheung-Flynn et al., 2005). Knockdown of FKBP4 expression in a human HeLa cell model reduced the effect of androgens on these cells via a reduction in androgen receptor expression (Yong et al., 2007; Cheung-Flynn et al., 2005). It is likely that through this mechanism, crosses between Fkbp4-null males with wild-type females fail to result in pregnancy (Hong et al., 2007, Cheung-Flynn et al., 2005). A decrease in the steady-state level of AR was also observed in the testis and epididymis of Fkbp-null mice. While this did not alter organogenesis of these tissues, it may result in reduced sperm motility and decreased fertilization rates (Cheung-Flynn et al., 2005). Abnormalities like those observed in Fkbp4 males have been observed in humans that produce inadequate androgen levels or that respond inadequately to androgens due to AR gene mutation (Miller, 2002).

Zinc finger protein 42 (ZFP42 aka Rex1) encodes a zinc finger protein which functions as a DNA-binding transcription factor. It is highly expressed in preimplantation embryos (Rogers et al., 1991), where it is likely to regulate ICM identity, due to the role of Rex1 in the regulation of pluripotency. It is also expressed in the placenta, and is only conserved among placental mammals (Kim et al., 2008). The protein sequence of Rex1 shares high levels of sequence identity with another C2H2 zinc finger protein YY1, which is expressed in the oocyte and required for follicle expansion (Griffith et al., 2011). However, about half of both homozygous and heterozygous Rex1 mice die during the late gestation and neonatal stages (Masui et al., 2008). This delayed phenotypic consequence suggests potential roles for Rex1 in establishing and maintaining unknown epigenetic modifications. Consistent with this, Rex1-null blastocysts display hypermethylation in the differentially methylated regions (DMRs) of Peg3 and Gnas imprinted domains, which are known to contain YY1 binding sites. Further analyses confirmed in vivo binding of Rex1 only to the unmethylated allele of these two regions. Thus, Rex1 may function as a protector for these DMRs against DNA methylation (Kim et al., 2008).

### Assays

Genotypic data can be obtained, for example, by conducting an assay on a sample from a male or female that detects either a mutation in an infertility-associated genetic region or abnormal (over or under) expression of an infertility-associated genetic region. The presence of certain mutations in those genetic regions or abnormal expression levels of those genetic regions is indicative of fertility outcomes, i.e., whether a pregnancy or live birth is achievable. Exemplary variants include, but are not limited to, a single nucleotide polymorphism, a deletion, an insertion, an inversion, a genetic rearrangement, a copy number variation, or a combination thereof.

A sample may include a human tissue or bodily fluid and may be collected in any clinically acceptable manner. A tissue is a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CSF. A sample may also be a fine needle aspirate or biopsied tissue, e.g. an endometrial aspirate, breast tissue biopsy, and the like. A sample also may be media containing cells or biological material. A sample may also be a blood clot, for example, a blood clot that has been obtained from whole blood after the serum has been removed. In certain embodiments, the sample may include reproductive cells or tissues, such as gametic cells, gonadal tissue, fertilized embryos, and placenta. In certain embodiments, the sample is blood, saliva, or semen collected from the subject.

Genotypic information from the sample can be obtained by nucleic acid extraction from the sample. Methods for extracting nucleic acid from a sample are known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, a sample is collected from a subject followed by enrichment for genes or gene fragments of interest, for example by hybridization to a nucleotide array including fertility-related genetic regions or genetic fragments of interest. The sample may be enriched for genetic regions of interest (e.g., infertility-associated genetic regions) using methods known in the art, such as hybrid capture. See for examples, Lapidus (U.S. Pat. No. 7,666,593), the content of which is incorporated by reference herein in its entirety.

RNA may be isolated from eukaryotic cells by procedures that involve lysis of the cells and denaturation of the proteins contained therein. Tissue of interest includes gametic cells, gonadal tissue, endometrial tissue, fertilized embryos, and placenta. Fluids of interest include blood, menstrual fluid, mammary fluid, follicular fluid of the ovary, peritoneal fluid, or culture medium. Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+ RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989)). Alternatively, separation of RNA from DNA can be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., CURRENT PROTOCOLS IN MOLECULAR BIOLOGY, vol. 2, Current Protocols Publishing, New York (1994). Once bound, poly(A)+ mRNA is eluted from the affinity column using 2 mM EDTA/0.1% SDS.

Detailed descriptions of conventional methods, such as those employed to make and use nucleic acid arrays, amplification primers, hybridization probes, and the like can be found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.). Applied Biosystems (Foster City, Calif.), and Agilent Technologies (Santa Clara, Calif.).

Methods of detecting variants in genetic regions are known in the art. In certain embodiments, a variant in a single infertility-associated genetic region indicates infertility. In other embodiments, the assay is conducted on more than one infertility-associated genetic regions (e.g., the genes from Table 2), and a variant in at least two infertility-associated genetic regions indicates infertility. In other embodiments, a variant in at least three infertility-associated genetic regions indicates infertility; a variant in at least four infertility-associated genetic regions indicates infertility; a variant in at least five infertility-associated genetic regions indicates infertility; a variant in at least six infertility-associated genetic regions indicates infertility; a variant in at least seven infertility-associated genetic regions indicates infertility; a variant in at least eight infertility-associated genetic regions indicates infertility; a variant in at least nine infertility-associated genetic regions indicates infertility; a variant in at least 10 infertility-associated genetic regions indicates infertility; a variant in at least 15, 20, 25, 30, 35, 50, 75, 100 or more, or any integer inbetween, infertility-associated genetic regions indicates infertility. In one embodiment, a variant in all of the genetic regions from Table 1 indicates infertility.

In certain embodiments, a known single nucleotide polymorphism at a particular position can be detected by single base extension for a primer that binds to the sample DNA adjacent to that position. See for example Shuber et al. (U.S. Pat. No. 6,566,101), the content of which is incorporated by reference herein in its entirety. In other embodiments, a hybridization probe might be employed that overlaps the SNP of interest and selectively hybridizes to sample nucleic acids containing a particular nucleotide at that position. See for example Shuber et al. (U.S. Pat. Nos. 6,214,558 and 6,300,077), the content of which is incorporated by reference herein in its entirety.

In particular embodiments, nucleic acids are sequenced in order to detect variants (i.e., mutations) in the nucleic acid compared to wild-type and/or non-mutated forms of the sequence. The nucleic acid can include a plurality of nucleic acids derived from a plurality of genetic elements. Methods of detecting sequence variants are known in the art, and sequence variants can be detected by any sequencing method known in the art e.g., ensemble sequencing or single molecule sequencing.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes

One conventional method to perform sequencing is by chain termination and gel separation, as described by Sanger et al., Proc Natl. Acad. Sci. USA, 74(12): 5463 67 (1977). Another conventional sequencing method involves chemical degradation of nucleic acid fragments. See, Maxam et al., Proc. Natl. Acad. Sci., 74: 560 564 (1977). Finally, methods have been developed based upon sequencing by hybridization. See. e.g., Harris et al., (U.S. patent application number 2009/0156412). The content of each reference is incorporated by reference herein in its entirety.

A sequencing technique that can be used in the methods of the provided invention includes, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslavsky, et al., PNAS (USA), 100: 3960-3964 (2003), the contents of each of these references is incorporated by reference herein in its entirety.

Another example of a DNA sequencing technique that can be used in the methods of the provided invention is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H⁺), which signal detected and recorded in a sequencing instrument. The signal strength is proportional to the number of nucleotides incorporated.

Another example of a sequencing technology that can be used in the methods of the provided invention is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using an electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

If the nucleic acid from the sample is degraded or only a minimal amount of nucleic acid can be obtained from the sample, PCR can be performed on the nucleic acid in order to obtain a sufficient amount of nucleic acid for sequencing (See e.g., Mullis et al. U.S. Pat. No. 4,683,195, the contents of which are incorporated by reference herein in its entirety).

In certain aspects, the invention provides a microarray including a plurality of oligonucleotides attached to a substrate at discrete addressable positions, in which at least one of the oligonucleotides hybridizes to a portion of a gene suspected of affecting fertility in a man or woman. Methods of constructing microarrays are known in the art. See for example Yeatman et al. (U.S. patent application number 2006/0195269), the content of which is hereby incorporated by reference in its entirety.

Microarrays are prepared by selecting probes that include a polynucleotide sequence, and then immobilizing such probes to a solid support or surface. The probe or probes used in the methods of the invention are preferably immobilized to a solid support which may be either porous or non-porous. See, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In preferred embodiments, a microarray comprises a support or surface with an ordered array of binding (e.g., hybridization) sites or “probes” each representing a fertility-associated gene, such as one of the genes described in Table 1. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the biomarkers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment. In one embodiment, probes representing each of the markers are present on the array. In a preferred embodiment, the array comprises probes for each of the genes listed in Table 1.

As noted above, the probe to which a particular polynucleotide molecule specifically hybridizes according to the invention contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR PROTOCOLS: A GUIDE TO METHODS AND APPLICATIONS, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983); Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In a particularly preferred embodiment, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells, which define the locations of the array elements (i.e., the different probes). Microarrays manufactured by this ink-jet method are typically of high density, preferably having a density of at least about 2,500 different probes per 1 cm² The polynucleotide probes are attached to the support covalently at either the 3′ or the 5′ end of the polynucleotide.

The polynucleotide molecules which may be analyzed by the present invention are DNA, RNA, or protein. The target polynucleotides are detectably labeled at one or more nucleotides. Any method known in the art may be used to detectably label the target polynucleotides. Preferably, this labeling incorporates the label uniformly along the length of the DNA or RNA, and more preferably, the labeling is carried out at a high degree of efficiency.

In a preferred embodiment, the detectable label is a luminescent label. For example, fluorescent labels, bioluminescent labels, chemiluminescent labels, and colorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a reference sample. The reference can comprise target polynucleotide molecules from normal tissue samples.

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B.V.; and Kricka, 1992, NONISOTOPIC DNA PROBE TECHNIQUES, Academic Press, San Diego, Calif.

Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled genes or gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as quantitative reverse transcription polymerase chain reaction (qRT-PCR) (Weis et al., Trends in Genetics 8:263 264 (1992), the contents of which are incorporated by reference herein in their entirety). Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

A differentially or abnormally expressed gene refers to a gene whose expression is activated to a higher or lower level in a subject suffering from a disorder, such as infertility, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as infertility, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure.

The first step is the isolation of mRNA from a target sample. The starting material is typically total RNA isolated from human tissues or fluids. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). The contents of each of these references are incorporated by reference herein in their entirety. In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE Complete DNA and RNA Purification Kit (EPICENTRE, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In certain embodiments, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and actin, beta (ACTB). For performing analysis on pre-implantation embryos and oocytes, conserved helix-loop-helix ubiquitous kinase (CHUK), UBC, HPRT, and H2AFZ are among genes that can be used for normalization.

A more recent variation of the RT-PCR technique is the real time quantitative PCR, which measures PCR product accumulation through a dual-labeled fluorogenic probe (i.e., TaqMan® probe). Real time PCR is compatible both with quantitative competitive PCR, in which internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996), the contents of which are incorporated by reference herein in their entirety.

In another embodiment, a MassARRAY-based gene expression profiling method is used to measure gene expression. In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059 3064 (2003).

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269), the content of which is incorporated by reference herein in its entirety.

In a specific embodiment of the microarray technique, PCR amplified inserts of cDNA clones are applied to a substrate in a dense array, for example, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996), the contents of which are incorporated by reference herein in their entirety). Microarray analysis can be performed by commercially available equipment, following manufacturer's protocols, such as by using the Affymetrix GenChip technology, or Incyte's microarray technology.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see. e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484 487 (1995); and Velculescu et al., Cell 88:243 51 (1997, the contents of each of which are incorporated by reference herein in their entirety).

In other embodiments Massively Parallel Signature Sequencing (MPSS) is used to measure gene expression. This method, described by Brenner et al., Nature Biotechnology 18:630 634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×106 microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the gene products of the present invention. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See, for example, U.S. Pat. Nos. 6,925,389; 6,989,100; and 6,890,763, each of which is incorporated by reference herein in their entirety.

### Identification of Genetic Loci Correlated with Fertility

As discussed above, genes of interest are not limited to those maternal effect genes listed in Table 1. Genes involved in all processes affecting fertility, for example but not limited to the processes shown in FIGS. 4-6, are contemplated herein. Methods and systems for identifying fertility-related genes of interest are also contemplated herein.

The invention provides applications and methods for determining the identity of genetic loci biologically or statistically correlated with fertility in an individual or a couple. In one aspect, the invention provides nucleic acid sequences that can be used to assess the presence or absence of particular nucleotides at polymorphic sites in an individual's RNA or genomic DNA that are associated with fertility. In certain aspects, the invention provides methods for observing commonly occurring or rare genetic variants within a subset of genes of interest for human infertility. In certain aspects, the invention provides methods for ranking the relative importance of individual genetic variants, genes, or genetic regions for allowing determination of infertility risk.

Whole genome sequencing (WGS) allows one to characterize the complete nucleic acid sequence of an individual's genome. With the amount of data obtained from WGS, a comprehensive collection of an individual's genetic variation is obtainable, which provides great potential for genetic biomarker discovery. The data obtained from WGS can be advantageously used to expand the ability to identify and characterize male and female infertility biomarkers. However, the ability to identify unknown variations of fertility significance within the vast WGS datasets is a challenging task that is analogous to finding a needle in a haystack.

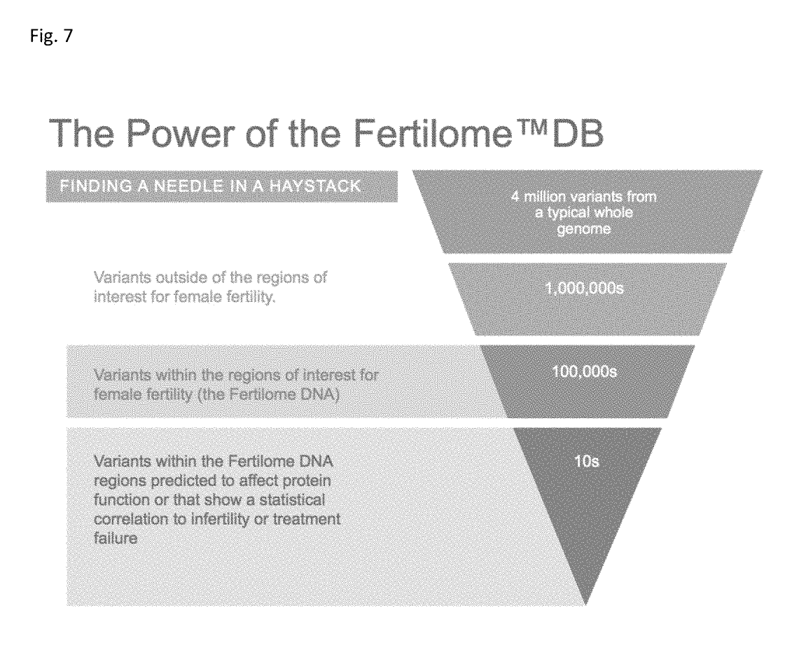

Methods of the invention, according to certain embodiments, rely on bioinformatics to filter through WGS data in order to identify and prioritize variations of infertility significance. Specifically, the invention relies on a combination of clinical phenotypic data and an infertility knowledgebase to rank and/or score genomic regions of interest and their likely impact on different fertility disorders. In certain aspects, the filtering approach involves assessing sequencing data to identify genomic variations, identifying at least one of the variations as being in a genomic region associated with infertility, determining whether the at least one variation is a biologically-significant variation and/or a statistically-significant variation, and characterizing at least one identified variation as an infertility biomarker based on the determining step. A genomic region associated with infertility is any DNA sequence in which variation is associated with a change in fertility. Such regions may include genes (e.g. any region of DNA encoding a functional product), genetic regions (e.g. regions including genes and intergenic regions with a particular focus on regions conserved throughout evolution in placental mammals), and gene products (e.g., RNA and protein). In particular embodiments, the infertility-associated genetic region is a maternal effect gene or any gene involved in the processes shown in FIGS. 7-9, as described above. In particular embodiments, the infertility-associated genetic region is a gene (including exons, introns, and evolutionarily conserved regions of DNA flanking either side of said gene) or region of non-coding DNA that affects the function of a gene or collection of genes, that impact(s) fertility.

This filtering approach facilitates rapid identification of functionally relevant variants within genomic regions of significance for fertility. The identified genetic variations with infertility significance obtained from WGS data may be used to further define an individual or couple's fertility profile, to assist in diagnostic testing, and ultimately to assist physicians in data interpretation, guide fertility therapeutics, and clarify why some patients are not responding to treatment. The following illustrates use of WGS data to identify variants of interest in accordance with methods of the invention. It is to be understood that the illustrated method can be expanded and/or modified to include regions of interest for male fertility and/or combined male and female fertility.

FIG. 7 generally illustrates filtering through variations obtained from WGS sequencing data in order to identify variations of infertility significance. As shown in FIG. 7, the first step is to identify sequence variants in whole genome sequence. A typical whole genome can include up to four million variants. The next filtering step involves eliminating variants outside of regions of interest for female fertility (which amounts to about one million variants). Next, the filtering method isolates variants within regions of interest for female fertility, which is described herein as Fertilome® nucleic acid (i.e. regions of the human genome that control egg quality and fertility). Variations located within the Fertilome® nucleic acid may be in the 100,000s. The variations within the Fertilome® nucleic acid are further filtered to identify and score variations of infertility significance (such variations are typically present in double digits). Particularly, variations of infertility significance include those within regions predicted to effect biological function or that show a statistical correlation to infertility or treatment failure. It is to be understood that the illustrated method can be expanded and/or modified to include regions of interest for male fertility and/or combined male and female fertility.

Biologically-significant variations within the Fertilome® nucleic acid include mutations that result in a change: 1) to a different amino acid predicted to alter the folding and/or structure of the encoded protein, 2) to a different amino acid occurring at a site with high evolutionarily conservation in mammals, 3) that introduces a premature stop termination signal, 4) that causes a stop termination signal to be lost, 5) that introduces a new start codon, 6) that causes a start codon to be lost or 7) that disrupts a splicing signal. Biologically significant variants can also include those that affect e.g. the promoter region of the gene, thereby affecting the ability of transcription factors and transcriptional machinery from binding to the promoter. This is among other examples of trans-regulatory elements.

Other methods for classifying variations as statistically- or biologically-significant includes scoring variations using an infertility knowledgebase which ranks genes based on attributes associated with infertility. The attributes include: diseases and disorders related to infertility, molecular pathways, molecular interactions, gene clusters, mouse phenotypes associated with each gene, gene expression data in reproductive tissues, proteomics data in oocytes, and accrued information from scientific publications through text-mining.

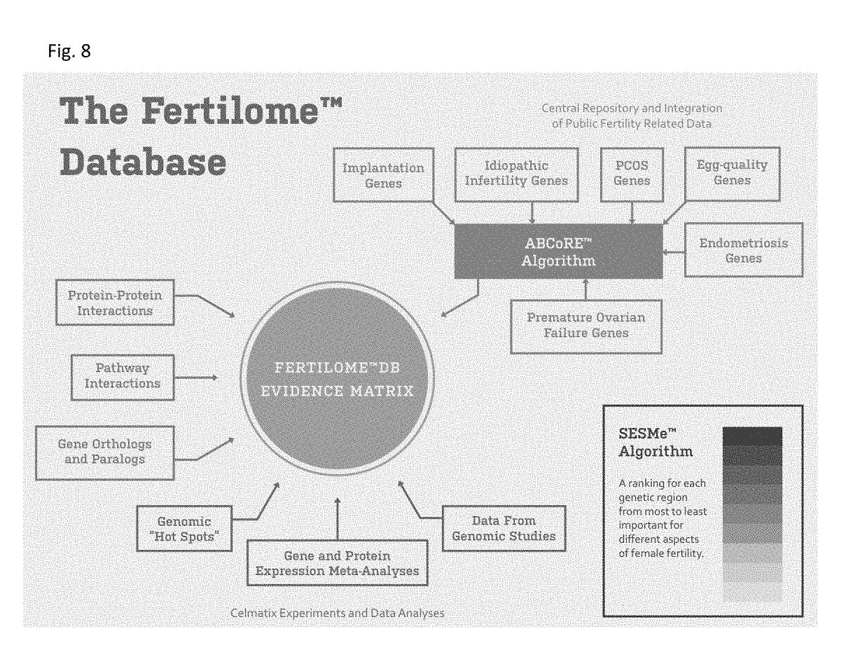

FIG. 8 illustrates various data sources that can be integrated into the infertility knowledgebase for analyzing whole-genome sequencing data according to certain embodiments. As shown in FIG. 8, information is obtained from private and public fertility-related data. Private and/or public fertility-related data may include implantation genes, idiopathic infertility genes, polycystic ovary syndrome (PCOS) genes, egg quality genes, endometriosis genes, and premature ovarian failure genes. Although not shown here, the data may also include those genes involved in male reproductive/fertility processes and other female reproductive/fertility processes. The private and/or public fertility-related data is then subjected to the ABCoRE Algorithm to provide genomic regions and variations of interest that can be introduced into a fertility database evidence matrix along with other fertility-related information. As described in more detail below, the ABCoRE algorithm identifies fertility regions of interest by performing evolutionary conservation analysis of one or more genes obtained from the private and/or public fertility-related data. The other fertility-related information includes, for example, protein-protein interactions, pathway interactions, gene orthologs and paralogs, genomic “hotspots”, gene protein expression and meta-analysis, and data from genomic studies. In operation, whole genomic sequencing data is compared to the compiled data in the fertility database evidence matrix to facilitate identification of potential genetic regions important for fertility. The fertility database evidence matrix filters through WGS variants to identify variants of fertility significance. In certain embodiments, the whole genomic sequencing data can also subjected to an algorithm that ranks each genetic region from most to least important for different aspects of male and female fertility. In one example, as shown in FIG. 8, the SESMe algorithm ranks each genetic region from most to least important for different aspects of female fertility, but can be expanded to include different aspects of male fertility as well.

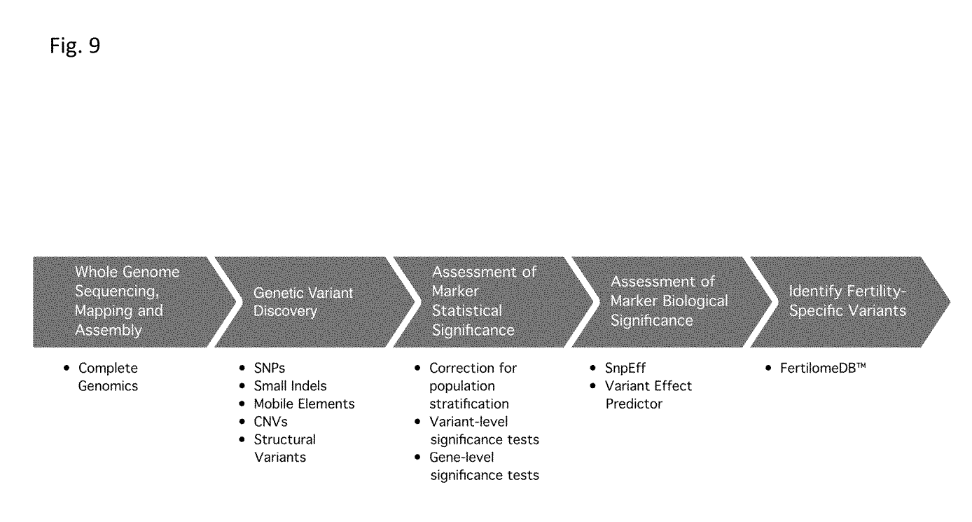

FIG. 9 illustrates a bioinformatics pipeline used to filter through WGS data to identify biomarkers associated with infertility according to certain embodiments. As shown in FIG. 9, samples are subjected to whole genome sequencing, mapping, and assembly. The WGS data is then analyzed to discover genetic variants such as SNPs, small indels, mobile elements, copy number variations, and structural variations. The identified variations are then assessed for statistical significance. This includes correction for population stratification, variation-level significance tests, and gene level significance tests. In addition, the biological significance of WGS variants is determined using the SnpEff and Variant Effect Predictor (www.ensembl.org) engines, in the case of variants within coding regions of DNA. Variants of known biological and/or statistical significance are then entered into an infertility knowledgebase (i.e. Fertilome® database) in order to classify those variants as fertility biomarkers.

According to certain aspects, methods of the invention provide for determining fertility/infertility genetic regions of interest based on data obtained from public and private fertility/infertility related databases. Infertility/fertility related data may include implantation genes, idiopathic infertility genes, polycystic ovary syndrome (PCOS) genes, egg quality genes, endometriosis genes, premature ovarian failure genes, other genes involved in female reproductive/fertility processes, and genes involved in male reproductive/fertility processes. As described below, the infertility/fertility related data can then be processed using evolutionary conservation to identify genomic regions and variations of interest.

Evolutionary conservation analysis involves, generally, comparing nucleic acid sequences among evolutionary and distantly related genomes to identify similarities and differences between coding and/or non-coding regions across the genomes. Conservation of coding and/or non-coding sequences is described in Hardison et al., W. 1997, Genome Res. 7: 959-966; Brenner et al., 2002, Proc. Natl. Acad. Sci. 99: 2936-2941; Karolchik et al., Comparative Genomics. Humana Press, 2008. 17-33; Santini et al., Genome research 13.6a (2003): 1111-1122; Roth et al., 1998, Nat. Biotechnol. 16: 939-945; and Blanchette, M. and Tompa, M. 2002, Genome Res. 12: 739-748. A degree of conservation (e.g. degree of similarity between a target genomic region and related genomes) that is considered to be functionally relevant depends on the particular application. For example, a functionally relevant degree of conservation may be 80%, 85%, 90%, 91%, 92%, 93%, 94%, 95%, 96% 97%, 98%, 99%, etc. Regions of genes identified by evolutionary conservation as being functionally-relevant can then be used as regions of interest for diagnosing diseases and disorders, such as infertility.

According to certain embodiments, infertility regions of interest are identified by performing evolutionary conservation analysis of one or more genes or genetic regions obtained from infertility and/or fertility-related data. The process of filtering through infertility/fertility related databases using evolutionary conservation, according to the invention, is called the ABCoRE algorithm (see FIG. 8). For example, nucleic acid data obtained from the infertility/fertility related databases can be compared to distantly related genomes in order to assess conservation of the infertility-related nucleic acid. Regions of the nucleic acid determined to be conserved are classified as infertility regions of interest.

In particular aspects, the following method is employed to determine whether a genomic region is a fertility region of interest using conservation analysis. First, private and/or public nucleic acid data corresponding to infertility or fertility is obtained. Next, one or more genetic loci from that data is examined for conservation. The coding regions (i.e. exons)) of a gene, non-coding regions of the gene, and/or regions flanking the gene (intergenic regions upstream and downstream from the gene being examined) are then analyzed for conservation. Coding, non-coding, and intergenic regions may be classified as an infertility region of interest if they have a degree of conservation of, for example, 80%, 85%, 90%, 91%, 92%, 93%, 94%, 95%, 96% 97%, 98%, 99%, etc.

Once genetic regions of interest are determined, the regions can then be ranked according to significance using any number of ranking schemes known in the art and/or one or more of the ranking schemes described below and in more detail in co-owned U.S. patent application Ser. No. 14/605,452, the contents of which are incorporated herein in its entirety.

In addition to ranking regions of interest determined through conservation analysis of private and public data as described above, genetic loci are ranked according to their expression levels in humans and mice. For example, in one aspect of an embodiment of the invention, it is determined whether a biomarker is expressed in mice. If the biomarker is expressed in mice, the biomarker receives a higher ranking. If the biomarker is also expressed in humans, the biomarker is ranked even higher by the ranking system. If a biomarker is not expressed in mice, or in humans, it would receive a low ranking. A biomarker would receive the lowest ranking if it was expressed neither in mouse nor in human.

Known methods in the art can be employed to rank genetic regions. It should be appreciated that any known ranking methodology can be utilized in the present invention, as discussed above. For example, the Friedman test. Kruskal-Wallis test. Spearman's rank correlation coefficient, Wilcoxon rank-sum test, and/or Wilcoxon signed-rank test are known statistical methods. The Friedman test is similar to the parametric repeated measures ANOVA; it is used to detect differences in treatments across multiple test attempts. The procedure involves ranking each row (or block) together, then considering the values of ranks by columns. See Friedman, Milton (December 1937). “The use of ranks to avoid the assumption of normality implicit in the analysis of variance”. Journal of the American Statistical Association (American Statistical Association) 32 (200): 675-701. Also, the Spearman's rank-order correlation is the nonparametric version of the Pearson product-moment correlation. Spearman's correlation coefficient measures the strength of association between two ranked variables. See Lehman, Ann (2005). Jmp For Basic Univariate And Multivariate Statistics: A Step-by-step Guide. Cary, N.C.: SAS Press. p. 123. The Wilcoxon signed-rank test is a non-parametric statistical hypothesis test used when comparing two related samples, matched samples, or repeated measurements on a single sample to assess whether their population mean ranks differ (i.e., it is a paired difference test). See Wilcoxon, Frank (December 1945). “Individual comparisons by ranking methods”. Biometrics Bulletin 1 (6): 80-83.

In one aspect of the invention, a first ranking scheme ranks genes according the number of variants that were predicted to significantly affect protein structure and function (biologically significant) out of a list of fertility genes. The most highly ranked genes contained the most variants. Genetic variants considered to be biologically significant include mutations that result in a change: 1) to a different amino acid predicted to alter the folding and/or structure of the encoded protein, 2) to a different amino acid occurring at a site with high evolutionarily conservation in mammals, 3) that introduces a premature stop termination signal, 4) that causes a stop termination signal to be lost, 5) that introduces a new start codon, 6) that causes a start codon to be lost, 7) that disrupts a splicing signal, 8) that alters the reading frame or 9) that alters the dosage of encoded protein or RNA. All genetic variants detected from re-sequencing exclude sites where the variant allele is detected in only one chromosome (singletons) and sites sequenced in only one individual.

A second ranking scheme ranks genes based on statistical significance of variants detected in the coding regions of the genes using a variant coding score. Genes can be ranked in order from most to least statistically significant. The statistical significance of a gene's correlation with infertility risk can be determined using the results of a study of unexplained female infertility based on variants detected in the coding regions of these genes. In one aspect, p-values <0.025 are considered statistically significant, such that fertility genes that do not meet this criteria are not ranked. For the coding level analysis, we a coding variant score for the coding regions for each individual/gene can be computed. The coding variant score represents the variability of the gene at coding regions in an individual and is computed as the sum of the proportion of variant locations within the coding regions of that gene for that individual. A series of linear regression models are fit, where the outcome variable is the coding variant score for a given gene, and the independent variables are group (infertile vs control) and principal component derived ethnicity (continuous). The p-value for group is used for statistical inference. The model is fit once for each gene. Additionally or alternatively, genes can be ranked in order from most to least statistically significant based on correlations with phenotype in mice. In this case, the outcome variable is the phenotype expression score for a given gene, and the independent variables are group (expressed phenotype v. control) and principal component derived ethnicity (for humans) or strain (for mice) (continuous).

A third ranking scheme is similar to the second ranking scheme noted above, except that it ranks genes based on statistical significance of variants detected in not only the coding regions, but also the non-coding, and conserved upstream and downstream regions of the fertility gene, using a gene variant score. In one aspect, p-values <0.025 are considered statistically significant, such that fertility genes that do not meet this criteria are not ranked. For the gene level analysis, a gene variant score is first computed for the entire transcript and flanking evolutionarily conserved regions for each individual/gene. The gene variant score represents the variability of the gene in an individual and is computed as the sum of the proportion of variant locations within that gene and its evolutionarily conserved regions flanking the gene for that individual. A series of linear regression models are fit, where the outcome variable is the gene variant score for a given gene, and the independent variables are group (infertile vs control) and principal component derived ethnicity (continuous). The p-value for group is used for statistical inference. The model is fit once for each gene.

A fourth ranking scheme ranks genes from most to least likely for variants in the gene to affect fertility, using a proprietary scoring model that reflects the likelihood that a gene is involved in fertility or reproduction. In one embodiment, genes can be ranked according to a Celmatix Fertilome® Score, G1Version2, that reflects the likelihood a gene is involved in fertility or reproduction. This score can be computed using a database of mined and curated data, containing attributes for each gene in the genome (See FIGS. 8 and 9). These attributes can include, but are not limited to: diseases and disorders related to infertility, molecular pathways, molecular interactions, gene clusters, mouse phenotypes associated with each gene, gene expression data in reproductive tissues, proteomics data in oocytes, and accrued information from scientific publications through text-mining.

One process for ranking fertility-related attributes of a gene or genetic region (locus) to obtain an infertility score is called the SESMe algorithm. The SESMe algorithm is applied to a database of features and attributes that might make a particular gene important for fertility. The algorithm assigns a score and a relative weight to each feature then ranks genetic regions from most to least important (or vice versa) by weighting features and attributes associated with that genetic region. For example, a score is assigned to a gene by compiling the combined weighted values of attributes associated with that gene. After each gene is scored based on its weighted attributes, the genes can be ranked in order of importance in accordance with their score. The weighted value for each infertility attribute may be scaled in any manner including and not limited to assigning a positive or negative integer to reflect the significance or severity of the attribute to infertility.

In certain embodiments, the weighted value for gene infertility attributes may be on a scale from −10 to +10. A +10 may indicate that an attribute of a gene being scored is highly associated with infertility because that attribute is prevalently found in infertile patient populations. A +4 may represent an attribute that is a latent infertility marker, meaning it will not cause infertility on its own, but may lead to infertility upon influence of external factors such as aging and smoking. Whereas +2 may represent an attribute found in some infertile patients but nothing directly relates the attribute to infertility. A zero on the scale may include an attribute not yet known to have any effect or any negative effect towards infertility. A −10 may include an attribute shown not to affect infertility whatsoever. Further, embodiments provide for the weighted scale to include a +1 for attributes that are commonly found in infertile patient populations, 0.5 for attributes similar to those found in infertile patient populations, and 0 for attributes without a causal link to infertility.

In addition, weighted values for attributes may be normalized based on the known significance of that attribute towards infertility. For example and in certain embodiments, when scoring attributes of a particular gene, each attribute may be assigned a 0 if the attribute is absent and a 1 if the attribute is present. The attributes may then be normalized based on the infertility significance of that attribute. For example, if the attribute is a genetic variant known to be associated with infertility, then that attribute may be normalized by a factor of 5. In another example, if the attribute is a signaling pathway defect sometimes associated with infertility, then that attribute may be normalized by a factor of 2.

A fifth ranking scheme ranks genes in the same manner as the fourth ranking scheme, except that it contains more fertility genes as an input for the score calculation (i.e., the Celmatix Fertilome™Score, G1Version3).

A sixth ranking scheme ranks genes according to how often a gene appears in one of the aforementioned five ranking schemes. A list of top 20 fertility-related genes in females obtained using this ranking scheme is provided in the table below, arranged in alphabetical order. It is also to be understood that the same scheme(s) can be used to rank fertility-related genes in males, as well as fertility-related genes in males and females combined.

TABLE 2

| Gene Symbol | Celmatix Gene ID | Entrez Gene ID | HGNC Gene ID |
|---|---|---|---|
| BARD1 | CMX-G0000004834 | 580 | 952 |
| C6orf221 | CMX-G0000010478 | 154288 | 33699 |
| DNMT1 | CMX-G0000026880 | 1786 | 2976 |
| FMR1 | CMX-G0000031614 | 2332 | 3775 |
| FOXO3 | CMX-G0000010672 | 2309 | 3821 |
| MUC4 | CMX-G0000006719 | 4585 | 7514 |
| NLRP11 | CMX-G0000028188 | 204801 | 22945 |
| NLRP14 | CMX-G0000016919 | 338323 | 22939 |
| NLRP5 | CMX-G0000028192 | 126206 | 21269 |
| NLRP8 | CMX-G0000028191 | 126205 | 22940 |
| NPM2 | CMX-G0000013114 | 10361 | 7930 |
| PADI6 | CMX-G0000000344 | 353238 | 20449 |
| PMS2 | CMX-G0000011251 | 5395 | 9122 |
| SCARB1 | CMX-G0000019991 | 949 | 1664 |
| SPIN1 | CMX-G0000014689 | 10927 | 11243 |
| TACC3 | CMX-G0000006818 | 10460 | 11524 |
| ZP1 | CMX-G0000017558 | 22917 | 13187 |
| ZP2 | CMX-G0000023549 | 7783 | 13188 |
| ZP3 | CMX-G0000011947 | 7784 | 13189 |
| ZP4 | CMX-G0000002903 | 57829 | 15770 |

All of the biologically and/or statistically significant variants detected in the genes depicted in Table 2 can be determined. Genetic variants considered to be biologically significant include mutations that result in a change: 1) to a different amino acid predicted to alter the folding and/or structure of the encoded protein, 2) to a different amino acid occurring at a highly evolutionarily conserved site, 3) that introduces a premature stop termination signal, 4) that causes a stop termination signal to be lost, 5) that introduces a new start codon, 6) that causes a start codon to be lost, 7) that disrupts a splicing signal, 8) that alters the reading frame or 9) that alters the dosage of encoded protein or RNA. Biologically significant variants can also include those that affect e.g. the promoter region of the gene, thereby affecting the ability of transcription factors and transcriptional machinery from binding to the promoter. This is among other examples of trans-regulatory elements. All genetic variants detected from resequencing exclude sites at the single nucleotide level where the variant allele is detected in only one chromosome (singletons) and sites sequenced in only one individual. Structural variants impacting biological function are also reported. Using these criteria applied to targeted re-sequencing data from a study of infertile females, we detected 490 variants. A list of these variants can be found in co-pending U.S. patent application Ser. No. 14/605,452, the contents of which are incorporated herein in its entirety.

For the statistically significant variant level analysis, a series of logistic regression models are fit, where the outcome variable is the binary indicator of variant status for a given location, and the independent variables are group (infertile vs. control) and principal component-derived ethnicity (continuous). The p-value and odds ratio for group are used for statistical inference. The model is fit once for each location. P-values <0.001 are considered statistically significant. We performed a SNP association study by targeted re-sequencing and identified a total of 147 SNPs significantly associated with female infertility (of which 52 are reported in Table 7 of co-pending U.S. patent application Ser. No. 14/605,452, incorporated herein in its entirety). Each variant was classified as novel or known. Novel sites are excluded from the p-value computation. For known variants, we apply a series of logistic regression models where the outcome variable is the binary indicator of variant status for a given location, and the independent variables are group (infertile vs. control) and principal component-derived ethnicity (continuous). The p-value and odds ratio for group are used for statistical inference. P-values less than 0.001 were considered significant.

In addition to using the existing infertility knowledge bases to identify new genetic variations associated with infertility, methods of the invention further utilize the existing infertility knowledgebase to identify commonalities between known infertility genes and genes having no prior association with infertility. By identifying commonalities between infertility genes and genes having no prior association with infertility, one is able to expand the list of potential genes associated with infertility and guide understanding as to what gene functions and changes are causally-linked to infertility. For example, genes having commonalities with known infertility genes can be identified as potential infertility biomarkers, and used in phenotypic studies (such those performed in mice) related to infertility, thereby expanding the breadth infertility knowledgebase.

In order to determine commonalities between infertility genes and genes without prior association with infertility, methods of the invention can utilize cluster analysis techniques. Generally, a cluster analysis involves grouping a set of objects in such a way that certain objects clustered in one group are more similar to each other than objects in another group or cluster. Methods of the invention cluster known infertility genes with genes not associated with infertility based on features such as gene expression, phenotype, and genetic pathways. From the cluster analysis, one can identify genes without prior association with infertility that exhibit features with a high degree of similarity (relatedness) to infertility genes. Those genes exhibiting a high degree of similarity (as shown through the cluster analysis) can be identified as a potential infertility biomarker. The genetic loci identified by cluster analysis can also be used in further phenotypic studies in mouse models, such that the clustering of particular genetic loci may provide an understanding of how variant(s) in the gene(s) of interest might bring about the molecular, cellular and physiological changes sufficient to affect particular aspects of infertility.

The following describes a clustering method used to identify a potential infertility biomarker in accordance with methods of the invention. The method is typically a computer-implemented method, e.g. utilizes a computer system that includes a processor and a computer readable storage medium. The processor of the computer system executes instructions obtained from the computer-readable storage device to perform the cluster analysis.

In accordance with to certain aspects, the method involves obtaining a gene data set that includes both known infertility genes and genes having no prior association with infertility. In certain embodiments, the gene data sets may be taken from known infertility databases, sequencing data obtained from patients, or sequencing data obtained from mouse modeling studies. The genes forming the cluster data set (those associated with infertility and those not known to be associated with infertility) are typically mammalian genes. The mammalian genes may correspond to mouse genes, human, genes, or a combination thereof. A cluster analysis is then performed on the gene data set to determine a relationship between the one or more genes not associated with infertility and the known infertility genes. If a gene not associated with infertility is shown to cluster with a known infertility gene, the method provides for identifying that gene as a potential infertility biomarker. If the gene not associated with infertility does not cluster with a known infertility gene, then that gene is less likely to be causally linked to infertility in the same/similar manner as that known infertility gene.

Methods of the invention assess several features (or parameters) of genes in order to determine commonalities and thus cluster genes not associated with infertility with known infertility genes based on the commonalities. In certain embodiments, those features include gene expression, phenotypes, gene pathways, and a combination thereof. One or more of those features can contribute to a gene's position in the clustering.

Feature data (such as gene expression, phenotype, gene pathway, etc.) is obtained for both known infertility genes and genes not known to be associated with infertility. Examples of feature data include functional annotation such as gene boundaries, exons, splice sites, areas of putative non-coding RNAs and other elements such as promoters or CpG islands and features associated with those regions such as tissue-specific transcriptional expression from multiple mammalian systems including mouse and human, transgenic mouse strain phenotypes, variants in genetic loci or genetic regions that have been associated with different human diseases, the relationship of particular genetic loci to particular molecular or cellular pathways, gene ontology, protein-protein interactions, and variants that have been observed. Some of the data is from public sources (e.g., mouse phenotypes) and some data is from research studies (e.g., non-public data related to mouse phenotypes and non-coding areas of interest or coding region variants observed in patients with infertility).

The feature and gene data is compiled to form a matrix that will be used to exhibit the cluster analysis. For example, the feature data is pre-processed to express each domain as a matrix with genetic loci in rows and features in columns (or vice versa). For domains with continuous values such as gene expression, the features are the individual tissues where gene expression was measured, and each value in the matrix (Xij) represents the expression of gene i in tissue j. For domains with categorical values such as phenotypes, the features are the individual phenotypes, and each value in the matrix (Xij) is a binary indicator representing whether gene i is associated with phenotype j. Each domain matrix has R rows and Ck columns. In one aspect, each domain matrix can then scaled so that each gene has mean 0 and standard deviation 1. All of the domain specific matrices can then be combined column-wise, giving a matrix with R rows and ΣCk columns. A distance metric can then be applied to each pair of rows and each pair of columns in the matrix. In certain embodiments, the distance metric is ‘Distance=1−correlation’. It is also understood that other standard distance metrics could be used (e.g. Euclidean). According to one aspect of the invention, the weighted correlation value is the Pearson correlation with higher weights applied to specific features (columns). Since interest is in infertility driven clustering, infertility/reproductive associated phenotypes and tissues are given higher weights in the correlation value and hence in the distance calculation. Alternate weights could be used to emphasize other aspects of the gene information. The resulting distance value is 0 for genetic loci with identical annotation, and 1 for completely uncorrelated annotation.

Standard hierarchical clustering is then used to cluster the rows and columns of the matrix in order to determine feature commonalities between known infertility genes and other genes. Various hierarchical clustering techniques are known in the art, and can be applied to methods of the invention for clustering infertility genes with genes not associated with infertility. Hierarchical clustering techniques are described in, for example, Sturn, Alexander, John Quackenbush, and Zlatko Trajanoski. “Genesis: cluster analysis of microarray data.” Bioinformatics 18.1 (2002): 207-208; Yeung, Ka Yee, and Walter L. Ruzzo. “Principal component analysis for clustering gene expression data.” Bioinformatics 17.9 (2001): 763-774; Eisen, Michael B., et al. “Cluster analysis and display of genome-wide expression patterns.” Proceedings of the National Academy of Sciences 95.25 (1998): 14863-14868. Generally, clustering involves comparing features of one or more genes, and categorizing the genes into one or more feature groups based on the comparison. After the comparison, the cluster analysis may further involve assigning a value to the categorized genes based on a degree of relatedness. For example, genes clustered together having highly similar or the same features may be assigned a high value (e.g. positive integer). The degree of relatedness may be highlighted on the resulting cluster matrix via colors, e.g. high degree of commonality being shown in red and low degree of commonality being shown in blue.

After a hierarchical clustering technique is applied to the gene/feature data, the gene clusters are displayed against certain feature categories (e.g. phenotype/gene expression ‘category’), which in turn were clustered to reflect commonality as a result of the hierarchical analysis. For example, particular phenotypes of female- or male-specific reproductive processes might be grouped into separate clusters, and phenotypes of embryo patterning, morphology and growth are grouped in a separate cluster, etc. The degree of relatedness or commonality between clustered genes (as determined by the cluster analysis) can then be highlighted on the resulting cluster matrix. For example, a first color may be used to indicate that the gene is associated with one very specific phenotype and/or is expressed at high levels in the associated tissue/physiological system indicated on the opposite axis; whereas a second color may be used to indicate that the gene is associated with a number of different and varied phenotypes and/or is expressed at low levels in the associated tissue.

By clustering genes into feature specific groups and color-coding genes with high degree of relatedness, the resulting cluster matrix of the invention advantageously allows for visualization of groups of genes that are strongly associated with phenotypes relating to particular tissues or physiological systems (i.e. clusters of interest). Thus, cluster matrices of the invention allow one to quickly identify genes without prior association with infertility as potential infertility biomarkers based on their shown association (cluster) with known infertility biomarkers. This clustering and identification of potential infertility biomarkers is done independently from and without correlating a gene's proximity with other genes within or location in a genomic region associated with infertility. As a result, clustering provides an additional method of identifying infertility genes of interest that can be used to complement other techniques for identifying infertility genes of interest.

Cluster analysis is also applicable to mouse modeling as it relates to identification and/or characterization of previously unknown infertility related genes or genetic regions of interest. This type of analysis can be used to highlight new genetic loci for further phenotypic study in mouse models, and can create knowledge of how particular genetic loci cluster together to provide understanding of how variant(s) in the gene(s) of interest might bring about the molecular, cellular and physiological changes sufficient to affect particular aspects of infertility in humans. Accordingly, in certain aspects, the invention provides for methods of producing a genetically-altered mouse having a gene knock-out to determine if the gene in question is implicated in an infertility-associated phenotype. Additionally, the invention provides genetically altered mice for testing therapeutic agents. In those embodiments, methods of the invention further involve administering a therapeutic agent to the mouse, and assessing the effect of the therapeutic agent on phenotype. A therapeutic agent that rescues the phenotype, i.e., returns or partially re-establishes the wild type fertility phenotype, is a good drug candidate.

Other aspects of the invention provide methods for assessing how a human genomic alteration is associated with an infertility, by analyzing the phenotype in a mouse. Those methods involve identifying a human genomic region whose function is known to be associated with human infertility but for which mechanistic insight might not be known. The methods additionally involve producing a genetically-modified mouse in which the genetic region whose function is associated with human infertility is altered. The mouse is then assessed for presence of the infertility phenotype. Mouse modeling as it relates to the present invention is further described in co-pending U.S. patent application Ser. No. 14/605,440, the contents of which are incorporated herein in its entirety.

### Phenotypic Traits/Environmental Exposures

In addition to genotypic data, aspects of the invention include obtaining information regarding a male and female's fertility-related phenotypic traits and environmental variables, in order to determine the fertility potential of the couple. Exemplary traits for both males and females are provided in Table 3 below.

TABLE 3
Phenotypic and environmental variables impacting fertility success

Cholesterol levels on different days of the menstrual cycle
Age of first menses for patient and female blood relatives (e.g. sisters, mother, grandmothers)
Age of menopause for female blood relatives (e.g. sisters, mother, grandmothers)
Number of previous pregnancies (biochemical/ectopic/clinical/fetal heart beat detected, live birth
outcomes), age at the time, and outcome for patient and female blood relatives (e.g. sisters, mother,
grandmothers)
Diagnosis of Polycystic Ovarian Syndrome
History of hydrosalpinx or tubal occlusion
History of endometriosis, pelvic pain, or painful periods
Cancer history/type of cancer/treatment/outcome for patient and female blood relatives (e.g. sisters, mother,
grandmothers)
Age that sexual activity began, current level of sexual activity
Smoking history for patient and blood relatives
Travel schedule/number of flying hours a year/time difference changes of more than 3 hours (Jetlag and
Flight-associated Radiation Exposure)
Nature of periods (length of menses, length of cycle)
Biological age (number of years since first menses)
Birth control use
Drug use (illegal or legal)
Body mass index (current, lowest ever, highest ever)
History of polyps
History of hormonal imbalance
History of amenorrhoea
History of eating disorders
Alcohol consumption by patient or blood relatives
Details of mother's pregnancy with patient (i.e. measures of uterine environment): any drugs taken,
smoking, alcohol, stress levels, exposure to plastics (i.e. Tupperware), composition of diet (see below)
Sleep patterns: number of hours a night, continuous/overall
Diet: meat, organic produce, vegetables, vitamin or other supplement consumption, dairy (full fat or
reduced fat), coffee/tea consumption, folic acid, sugar (complex, artificial, simple), processed food versus
home cooked.
Exposure to plastics: microwave in plastic, cook with plastic, store food in plastic, plastic water or coffee
mugs.
Water consumption: amount per day, format: straight from the tap, bottled water (plastic or bottle), filtered
(type: e.g. Britta/Pur)
Residence history starting with mother's pregnancy: location/duration
Environmental exposure to potential toxins for different regions (extracted from government monitoring
databases)
Health metrics: autoimmune disease, chronic illness/condition
Pelvic surgery history
Life time number of pelvic X-rays
History of sexually transmitted infections: type/treatment/outcome
Female reproductive hormone levels: follicle stimulating hormone, anti-Mullerian hormone, estrogen,
progesterone
Stress
Thickness and type of endometrium throughout the menstrual cycle.
Age
Height
Fertility treatment history and details: history of hormone stimulation, brand of drugs used, basal antral
follicle count, follicle count after stimulation with different protocols, number/quality/stage of retrieved
oocytes/development profile of embryos resulting from in vitro insemination (natural or ICSI), details of
IVF procedure (which clinic, doctor/embryologist at clinic, assisted hatching, fresh or thawed
oocytes/embryos, embryo transfer (blood on the catheter/squirt detection and direction on ultrasound),
number of successful and unsuccessful IVF attempts
Morning sickness during pregnancy
Breast size before/during/after pregnancy
History of ovarian cysts
Twin or sibling from multiple birth (mono-zygotic or di-zygotic)
Semen analysis (count, motility, morphology)
Vasectomy
Testosterone levels
Date of last use and/or frequency of use of a hot tub or sauna
Blood type
DES exposure in utero
Past and current exercise/athletic history
Levels of phthalates, including metabolites:
MEP - monoethyl phthalate, MECPP - mono(2-ethyl-5-carboxypentyl) phthalate, MEHHP - mono(2-ethyl-
5-hydroxyhexyl) phthalate, MEOHP - mono(2-ethyl-5-ox-ohexyl) phthalate, MBP - monobutyl phthalate,
MBzP - monobenzyl phthalate, MEHP - mono(2-ethylhexyl) phthalate, MiBP - mono-isobutyl phthalate,
MCPP - mono(3-carboxypropyl) phthalate, MCOP - monocarboxyisooctyl phthalate, MCNP -
monocarboxyisononyl phthalate
Familial history of Premature Ovarian Failure/Insufficiency
Autoimmunity history - Antiadrenal antibodies (anti-21-hydroxylase antibodies), antiovarian antibodies,
antithyroid anitibodies (anti-thyroid peroxidase, antithyroglobulin)
Additional female hormone levels: Leutenizing hormone (using immunofluorometric assay), Δ4-
Androstenedione (using radioimmunoassay), Dehydroepiandrosterone (using radioimmunoassay), and
Inhibin B (commercial ELISA)
Number of years trying to conceive
Dioxin and PVC exposure
Hair color
Nevi (moles)
Lead, cadmium, and other heavy metal exposure
For a particular ART cycle: the percentage of eggs that were abnormally fertilized, if assisted hatching was
performed, if anesthesia was used, average number of cells contained by the embryo at the time of
cryopreservation, average degree of expansion for blastocyst represented as a score, average degree of
expansion of a previously frozen embryo represented as a score, embryo quality metrics including but not
limited to degree of cell fragmentation and visualization of a or organization/number of cells contained in
the inner cell mass (ICM), the fraction of overall embryos that make it to the blastocyst stage of
development, the number of embryos that make it to the blastocyst stage of development, use of birth
control, the brand name of the hormones used in ovulation induction, hyperstimulation syndrome, reason
for cancelation of a treatment cycle, chemical pregnancy detected, clinical pregnancy detected, count of
germinal vesicle containing oocytes upon retrieval, count of metaphase I stage eggs upon retrieval, count of
metaphase II stage eggs upon retrieval, count of embryos or oocytes arrested in development and the stage
of development or day of development post oocyte retrieval, number of embryos transferred and date in
days post-oocyte retrieval that the embryos were transferred, how many embryos were cryopreserved and at
what stage of development

Information regarding the fertility-associated phenotypic traits, such as those listed in Table 3, can be obtained by any means known in the art. In many cases, such information can be obtained from a questionnaire completed by the subject that contains questions regarding certain fertility-associated phenotypic traits. Additional information can be obtained from a questionnaire completed by the subject's partner and blood relatives. The questionnaire includes questions regarding the subject's environmental exposures, which may affect their fertility, such as his or her smoking habits or frequency of alcohol consumption. Information can also be obtained from the medical history of the subject, as well as the medical history of blood relatives and other family members. Additional information can be obtained from the medical history and family medical history of the subject's partner. Medical history information can be obtained through analysis of electronic medical records, paper medical records, a series of questions about medical history included in the questionnaire, and a combination thereof.

In other embodiments, information useful for determining a couple's fertility profile, both genetic and phenotypic, can be obtained by analyzing a sample collected from one or more of the male subject, female subject, blood relatives of the subject(s), gamete or embryo donors involved in the pregnancy effort, pregnancy surrogates, and a combination thereof, as described above. With respect to genotypic information, methods of the invention involve obtaining a sample that is suspected to include an infertility-associated gene or gene product.

In other embodiments, an assay specific to a phenotypic trait or an environmental exposure of interest is used. Such assays are known to those of skill in the art, and may be used with methods of the invention. For example, the hormones used in birth control pills (estrogen and progesterone) may be detected from a urine or blood test. Venners et al. (Hum. Reprod. 21(9): 2272-2280, 2006) reports assays for detecting estrogen and progesterone in urine and blood samples. Venner also reports assays for detecting the chemicals used in fertility treatments.

Similarly, illicit drug use may be detected from a tissue or body fluid, such as hair, urine, sweat, or blood, and there are numerous commercially available assays (LabCorp) for conducting such tests. Standard drug tests look for ten different classes of drugs, and the test is commercially known as a “10-panel urine screen”. The 10-panel urine screen consists of the following: 1. Amphetamines (including Methamphetamine) 2. Barbiturates 3. Benzodiazepines 4. Cannabinoids (THC) 5. Cocaine 6. Methadone 7. Methaqualone 8. Opiates (Codeine, Morphine, Heroin. Oxycodone, Vicodin, etc.) 9. Phencyclidine (PCP) 10. Propoxyphene. Use of alcohol can also be detected by such tests.

Numerous assays can be used to tests a patient's exposure to plastics (e.g., Bisphenol A (BPA)). BPA is most commonly found as a component of polycarbonates (about 74% of total BPA produced) and in the production of epoxy resins (about 20%). As well as being found in a myriad of products including plastic food and beverage contains (including baby and water bottles), BPA is also commonly found in various household appliances, electronics, sports safety equipment, adhesives, cash register receipts, medical devices, eyeglass lenses, water supply pipes, and many other products. Assays for testing blood, sweat, or urine for presence of BPA are described, for example, in Genuis et al. (Journal of Environmental and Public Health, Volume 2012, Article ID 185731, 10 pages, 2012).

### Prognosis Predictor/Statistical Analysis

In one embodiment of the invention, the information collected from the male and female subject can then be compared to a reference set of data in order to provide a fertility profile. In certain aspects, the reference set includes fertility-related data collected from a plurality of women and men. For example, in females, such data may include the fertility-associated phenotypic traits of the women, fertility-associated medical interventions, and their pregnancy outcome, i.e., whether or not a pregnancy or live-birth was achieved, per cycle of the selected reproductive method. Information collected from the men and women from the reference set can include any number of phenotypic traits and/or environmental exposures listed in Table 3, such as age, smoking habits, alcohol intake, and fertility-associated traits, etc. Information can be obtained by any means known in the art, some of which are described above. Additional details for preparing a mass data set for use, for example, in IVF studies are provided in Malizia et al., Cumulative live-birth rates after in vitro fertilization, N Engl J Med 2009; 360: 236-43, incorporated by reference herein in its entirety.

The invention provides methods and systems for determining the fertility potential of a male and female combined based on the male and female's fertility-associated phenotypic traits and/or genotypic data. In some embodiments, methods and systems of the invention use a prognosis predictor for determining the fertility potential. The prognosis predictor can be based on any appropriate pattern recognition method that receives input data representative of a plurality of fertility-associated genotypic and phenotypic traits and generates a fertility profile for the couple. The prognosis predictor can be trained with training data from a plurality of men and women for whom fertility-associated phenotypic traits, fertility-associated genetic variants, fertility-associated medical interventions, and pregnancy outcomes are known. The plurality of men and women used to train the prognosis predictor is also known as the training population. Various prognosis predictors that can be used in conjunction with the present invention are described below. In some embodiments, additional men and women having known trait profiles and pregnancy outcomes can be used to test the accuracy of the prognosis predictor obtained using the training population. Such additional patients are known as the testing population.

In certain embodiments, the methods of invention use a prognosis predictor, also called a classifier, for determining the fertility potential of a female and male combined. As noted above, the prognosis predictor can be based on any appropriate pattern recognition method that receives a plurality of fertility-associated characteristics, such as genotypic data and phenotypic traits, and provides an output comprising data indicating a prognosis, i.e., a couple's fertility potential. As discussed previously, the data can be obtained by completion of a questionnaire containing questions regarding certain fertility-associated phenotypic traits and/or the collection of a biological sample to obtain genotypic data or a combination thereof.

In one embodiment, the prognosis predictor can be prepared by (a) generating a reference set of men and women for whom fertility-associated characteristics, such as genotypic data and phenotypic traits, are known; (b) determining for each characteristic, a metric of correlation between the characteristic and a fertility outcome in a plurality of men and women having known fertility outcomes; (c) selecting one or more characteristics based on said level of correlation; (d) training a prognosis predictor, in which the prognosis predictor receives data representative of the characteristics selected in the prior step and provides an output indicating a fertility potential, with training data from the reference set of subjects including assessments of characteristics taken from the men and women.

Various known association analysis and statistical pattern recognition methods can be used in conjunction with the present invention. Suitable methods include, without limitation, logic regression, ordinal logistic regression, linear or quadratic discriminant analysis, clustering, principal component analysis, nearest neighbor classifier analysis, and Cox proportional hazards regression.

Association studies can be performed to analyze the effect of genetic variants or abnormal gene expression on a particular trait being studied, or any number of phenotypic traits and/or environmental exposures, such as those listed in Table 3 above. Infertility as a trait may be analyzed as a non-continuous variable in a case-control study that includes as the patients infertile males and/or females and as controls fertile males and/or females that are age and ethnically matched. Methods including logistic regression analysis and chi square tests may be used to identify an association between genetic variants or abnormal gene expression and infertility. In addition, when using logistic regression, adjustments for covariates like age, smoking, BMI and other factors that affect infertility, such as those shown in Table 3, may be included in the analysis.

In addition, haplotype effects can be estimated using programs such as Haploscore. Alternatively, programs such as Haploview and Phase can be used to estimate haplotype frequencies and then further analysis such as Chi square test can be performed. Logistic regression analysis may be used to generate an odds ratio and relative risk for each genetic variant or variants.

The association between genetic variants and/or abnormal gene expression and infertility may be analyzed within cases only or comparing cases and controls using analysis of variance. Such analysis may include, for example, adjustments for covariates like age, smoking, BMI and other factors that effect infertility. In addition, haplotype effects can be estimated using programs such as Haploscore.

Method of logistic regression are described, for example in, Ruczinski (Journal of Computational and Graphical Statistics 12:475-512, 2003); Agresti (An Introduction to Categorical Data Analysis, John Wiley & Sons, Inc., 1996, New York. Chapter 8); and Yeatman et al. (U.S. patent application number 2006/0195269), the content of each of which is hereby incorporated by reference in its entirety.

Other algorithms for analyzing associations are known. For example, the stochastic gradient boosting is used to generate multiple additive regression tree (MART) models to predict a range of outcome probabilities. Each tree is a recursive graph of decisions the possible consequences of which partition patient parameters; each node represents a question (e.g., is the FSH level greater than x?) and the branch taken from that node represents the decision made (e.g. yes or no). The choice of question corresponding to each node is automated. A MART model is the weighted sum of iteratively produced regression trees. At each iteration, a regression tree is fitted according to a criterion in which the samples more involved in the prediction error are given priority. This tree is added to the existing trees, the prediction error is recalculated, and the cycle continues, leading to a progressive refinement of the prediction. The strengths of this method include analysis of many variables without knowledge of their complex interactions beforehand.

A different approach called the generalized linear model, expresses the outcome as a weighted sum of functions of the predictor variables. The weights are calculated based on least squares or Bayesian methods to minimize the prediction error on the training set. A predictor's weight reveals the effect of changing that predictor, while holding the others constant, on the outcome. In cases where one or more predictors are highly correlated, in a phenomenon known as collinearity, the relative values of their weights are less meaningful; steps must be taken to remove that collinearity, such as by excluding the nearly redundant variables from the model. Thus, when properly interpreted, the weights express the relative importance of the predictors. Less general formulations of the generalized linear model include linear regression, multiple regression, and multifactor logistic regression models, and are highly used in the medical community as clinical predictors.

In one aspect of the invention, the genetic variants determined from a female and male subject and phenotypic and/or environmental data from the male and female subjects are accepted as input data, variables predictive of infertility from genetic, infertility-associated phenotypic and environmental exposure data and obtained from a reference set of males and females are identified, weighted predictor variables based on a magnitude of change in fertility attributed to each predictor variable are generated, and the weighted predictor variables can then be applied to the to the input data to generate a fertility profile that reflects the fertility potential of the male and the female combined.

Further non-limiting examples of implementing particular prognosis predictors are provided herein to demonstrate the implementation of statistical methods in conjunction with the training set.

In some embodiments, the analysis is based on a regression model, preferably a logistic regression model. Such a regression model includes a coefficient for each of the markers in a selected set of markers of the invention. In such embodiments, the coefficients for the regression model are computed using, for example, a maximum likelihood approach.

Cox proportional hazards regression also includes a coefficient for each of the markers in a selected set of markers of the invention. Cox proportional hazards regression incorporates censored data (women in the reference set that did not return for treatment). In such embodiments, the coefficients for the regression model are computed using, for example, a maximum partial likelihood approach.

Some embodiments of the present invention provide generalizations of the logistic regression model that handle multicategory (polychotomous) responses. Such embodiments can be used to discriminate an organism into one or three or more prognosis groups. Such regression models use multicategory logit models that simultaneously refer to all pairs of categories, and describe the odds of response in one category instead of another. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference.

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In the present invention, the selected fertility-associated phenotypic traits serve as the requisite continuous independent variables. The prognosis group classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how selected fertility-associated phenotypic trait manifests in the two groups (e.g., a group that achieves pregnancy and a group that does not) and how the selected trait correlates with the manifestation of other traits. For example, LDA can be applied to the data matrix of the N members in the training sample by K genes in a combination of genes described in the present invention. Then, the linear discriminant of each member of the training population is plotted. Ideally, those members of the training population representing a first subgroup (e.g. those subjects that do not achieve pregnancy) will cluster into one range of linear discriminant values (e.g., negative) and those member of the training population representing a second subgroup (e.g. those subjects that achieve pregnancy) will cluster into a second range of linear discriminant values (e.g., positive). The LDA is considered more successful when the separation between the clusters of discriminant values is larger. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, New York.

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

In some embodiments of the present invention, decision trees are used to classify patients using expression data for a selected set of molecular markers of the invention. Decision tree algorithms belong to the class of supervised learning algorithms. The aim of a decision tree is to induce a classifier (a tree) from real-world example data. This tree can be used to classify unseen examples which have not been used to derive the decision tree.

A decision tree is derived from training data. An example contains values for the different attributes and what class the example belongs. In one embodiment, the training data is data representative of a plurality of fertility-associated characteristics, such as genotypic data and phenotypic traits.

The following algorithm describes a decision tree derivation:

### Tree(Examples,Class,Attributes)

Create a root node
If all Examples have the same Class value, give the root this label
Else if Attributes is empty label the root according to the most common value
Else begin
Calculate the information gain for each attribute
Select the attribute A with highest information gain and make this the root attribute
For each possible value, v, of this attribute
Add a new branch below the root, corresponding to A=v
Let Examples(v) be those examples with A=v
If Examples(v) is empty, make the new branch a leaf node labeled with the most common value among Examples
Else let the new branch be the tree created by

### Tree(Examples(v),Class,Attributes-{A})

end

A more detailed description of the calculation of information gain is shown in the following. If the possible classes vi of the examples have probabilities P(vi) then the information content I of the actual answer is given by:

I(P(v₁), . . . ,P(vₙ))=nΣi=1−P(vᵢ)log₂ P(vᵢ)

The I-value shows how much information we need in order to be able to describe the outcome of a classification for the specific dataset used. Supposing that the dataset contains p positive (e.g. pregnancy achievers) and n negative (e.g. pregnancy non-achievers) examples (e.g. individuals), the information contained in a correct answer is:

I(p/p+n,n/p+n)=−p/p+n log₂ p/p+n−n/p+n log₂ n/p+n

where log₂ is the logarithm using base two. By testing single attributes the amount of information needed to make a correct classification can be reduced. The remainder for a specific attribute A (e.g. a trait) shows how much the information that is needed can be reduced.

Remainder(A)=vΣi=1pᵢ+n/p+nI(pᵢ/pi+nᵢ,nᵢ/pᵢ+nᵢ)

“v” is the number of unique attribute values for attribute A in a certain dataset, “i” is a certain attribute value, “pᵢ” is the number of examples for attribute A where the classification is positive (e.g. pregnancy achiever), “nᵢ” is the number of examples for attribute A where the classification is negative (e.g., pregnancy non-achiever).

The information gain of a specific attribute A is calculated as the difference between the information content for the classes and the remainder of attribute A:

Gain(A)=I(p/p+n,n/p+n)−Remainder(A)

The information gain is used to evaluate how important the different attributes are for the classification (how well they split up the examples), and the attribute with the highest information.

In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, cut are not limited to classification and regression trees (CART), multivariate decision trees, ID3, and C4.5.

In one approach, when an exemplary embodiment of a decision tree is used, the data representative of a plurality of fertility-associated characteristics across a training population is standardized to have mean zero and unit variance. The members of the training population are randomly divided into a training set and a test set. For example, in one embodiment, two thirds of the members of the training population are placed in the training set and one third of the members of the training population are placed in the test set. The expression values for a select combination of traits are used to construct the decision tree. Then, the ability for the decision tree to correctly classify members in the test set is determined. In some embodiments, this computation is performed several times for a given combination of molecular markers. In each iteration of the computation, the members of the training population are randomly assigned to the training set and the test set. Then, the quality of the combination of traits is taken as the average of each such iteration of the decision tree computation.

In some embodiments, the fertility-associated characteristics are used to cluster a training set. For example, consider the case in which ten genes described in the present invention are used. Each member m of the training population will have expression values for each of the ten genes. Such values from a member m in the training population define the vector:

X₁ₘ X₂ₘ X₃ₘ X₄ₘ X₅ₘ X₆ₘ X₇ₘ X₈ₘ X₉ₘ X₁₀ₘ

where Xᵢₘ is the expression level of the i^{th} gene in organism m. If there are m organisms in the training set, selection of i genes will define m vectors. Note that the methods of the present invention do not require that each the expression value of every single trait used in the vectors be represented in every single vector m. In other words, data from a subject in which one of the ith traits is not found can still be used for clustering. In such instances, the missing expression value is assigned either a “zero” or some other normalized value. In some embodiments, prior to clustering, the trait expression values are normalized to have a mean value of zero and unit variance.

Those members of the training population that exhibit similar expression patterns across the training group will tend to cluster together. A particular combination of traits of the present invention is considered to be a good classifier in this aspect of the invention when the vectors cluster into the trait groups found in the training population. For instance, if the training population includes patients with good or poor prognosis, a clustering classifier will cluster the population into two groups, with each group uniquely representing either good or poor prognosis.

Clustering, as described above, and as described on pages 211-256 of Duda and Hart, Pattern Classification and Scene Analysis, 1973, John Wiley & Sons, Inc., New York; Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J., can also be used to find natural groupings. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Nearest neighbor classifiers are memory-based and require no model to be fit. Given a query point x₀, the k training points x₍ᵣ₎, r, . . . , k closest in distance to x₀ are identified and then the point x₀ is classified using the k nearest neighbors. Ties can be broken at random. In some embodiments, Euclidean distance in feature space is used to determine distance as:

d₍ᵢ₎=∥x₍ᵢ₎−xₒ∥.

Typically, when the nearest neighbor algorithm is used, the expression data used to compute the linear discriminant is standardized to have mean zero and variance 1. In the present invention, the members of the training population are randomly divided into a training set and a test set. For example, in one embodiment, two thirds of the members of the training population are placed in the training set and one third of the members of the training population are placed in the test set. Profiles represent the feature space into which members of the test set are plotted. Next, the ability of the training set to correctly characterize the members of the test set is computed. In some embodiments, nearest neighbor computation is performed several times for a given combination of fertility-associated phenotypic traits. In each iteration of the computation, the members of the training population are randomly assigned to the training set and the test set. Then, the quality of the combination of traits is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York.

The pattern classification and statistical techniques described above are merely examples of the types of models that can be used to construct a model for classification. It is to be understood that any statistical method can be used in accordance with the invention. Moreover, combinations of these described above also can be used. Further detail on other statistical methods and their implementation are described in co-owned U.S. patent application Ser. No. 11/134,688, the contents of which are incorporated by reference herein in their entirety.

With specific respect to women that make-up the reference set that may drop out prior to achieving a pregnancy or a live birth, it is not known whether those women eventually achieve a pregnancy at some later point or if they never became pregnant. However, simply omitting those women from the reference set would result bias to the reference data set by omitting characteristics of women having a poor prognosis of achieving a pregnancy or a live-birth. Such a bias would result in reporting an overly optimistic fertility potential and/or probability of achieving a pregnancy or live birth.

With systems and methods of the invention, rather than omitting those subjects wholesale, the present invention takes advantage of certain methods of statistical analysis to account for dropouts. The Kaplan-Meier method, for example, can be used to censor or exclude data for women in the reference set that dropped out. Other forms of statistical analysis can be used in accordance with the present invention to compile the data of the reference set. For example, logistic regression, ordinal logistic regression, Cox proportional hazards regression, and other methods can all be used to compile the data within the reference set. In addition, it is contemplated that the reference set can censor or account for dropouts based on the fertility-associated characteristics of the men and women rather than making blanket assumptions regarding the fertility status of the dropouts. For example, rather than simply assuming that a dropout had the same chance of becoming pregnant as the women who continued treatment, or assuming that a dropout had no chance of becoming pregnant, the present invention can evaluate the fertility-associated characteristics of the dropouts and informatively censor the dropouts based on such information. In this manner, overly-optimistic estimates (resulting from the assumption that all dropouts had equal chances of achieving live birth) or overly-conservative estimates (resulting from the assumption that the dropouts had no chances of achieving live birth) are avoided.

In certain aspects, the present invention incorporates the use of artificial censoring to account for dropouts. In artificial censoring, participants are censored when they meet a predefined study criterion, such as exposure to an intervention, noncompliance with their treatment regimen, or the occurrence of a competing outcome. Further analytical methods, such as inverse-probability-of-censoring weights (IPCW), can then be used to determine what the survival experiences of the artificially censored participants would have been had they never been exposed to the intervention, complied, or not developed the competing outcome. In some embodiments, methods encompassing the use of artificial censoring and further, the use of IPCW are encompassed by the invention to account for dropouts in the reference set. Additional detail regarding the use of artificial censoring and the use of IPCW is described in Howe et al., Limitation of inverse probability-of-censoring weights in estimating survival in the presence of strong selection bias, Am J Epidemiology, 2011, incorporated by reference herein in its entirety.

As mentioned above, the information collected from the male and female subjects is run through an algorithm trained on the reference set of data in order to provide a fertility potential. If the couple is currently undergoing fertility treatments, such as assisted reproductive technology procedures (e.g., IVF), the prognosis predictor can also be used to provide a fertility profile/probability of pregnancy for a selected cycle of treatment. The outcomes per cycle of treatment for the matched characteristics can then be identified. Based on the identified outcomes, the fertility profile/probability of pregnancy for the couple for a given cycle of treatment is provided. Various statistical models, as discussed above, can be used in accordance with the invention to improve the accuracy of the determination.

In further aspects of the invention, the fertility-associated characteristics within the reference set that are assessed for determining the fertility profile and/or probability of achieving a pregnancy are adjusted per cycle of treatment. For example, in a first round of in vitro fertilization, a woman's drinking or smoking habits may be especially relevant. In a later round, however, a women's age may be more pertinent. Accordingly, aspects of the invention encompass adjusting the assessed fertility-associated characteristics per cycle of treatment. Methods of the invention also include adjusting the assessed fertility-associated characteristics according to the selected fertility-associated medical intervention. For example, if IVF is the selected procedure, the condition of the woman's uterus may be more important than in ZIFT, which uses the Fallopian tubes rather than the uterus for implantation. A more detailed description of this aspect of the invention, and other aspects of the prognosis predictor, can be found in co-owned U.S. patent application Ser. No. 14/051,716 and U.S. Pat. No. 9,177,098, both of which are incorporated in their entirety herein.

### Systems

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

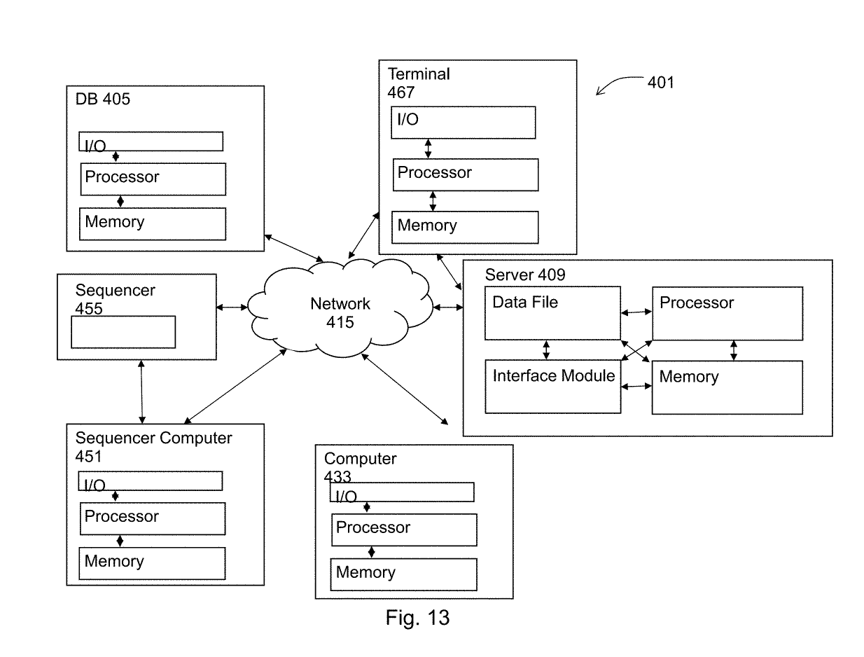

In an exemplary embodiment shown in FIG. 13, system 401 can include a computer 433 (e.g., laptop, desktop, or tablet). The computer 433 may be configured to communicate across a network 415. Computer 433 includes one or more processor and memory as well as an input/output mechanism. Where methods of the invention employ a client/server architecture, any steps of methods of the invention may be performed using server 409, which includes one or more of processor and memory, capable of obtaining data, instructions, etc., or providing results via interface module or providing results as a file. Server 409 may be engaged over network 415 through computer 433 or terminal 467, or server 415 may be directly connected to terminal 467, including one or more processor and memory, as well as input/output mechanism. In some embodiments, systems include an instrument 455 for obtaining sequencing data, which may be coupled to a sequencer computer 451 for initial processing of sequence reads

Memory according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

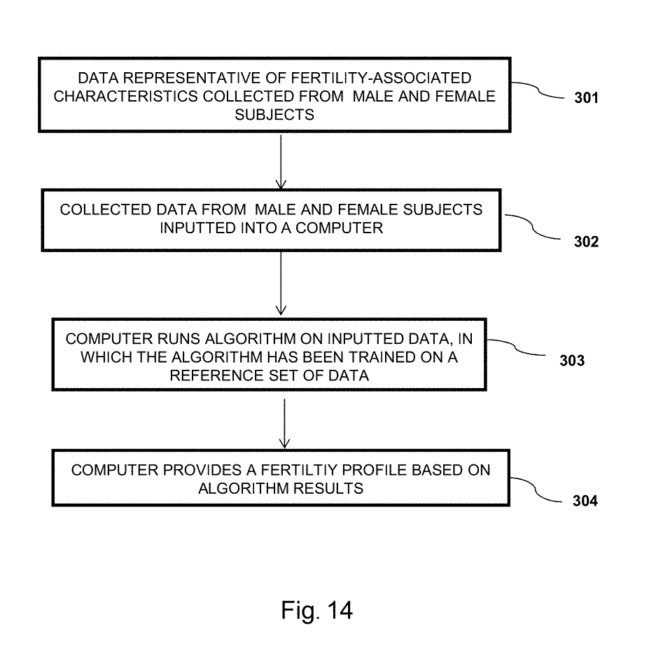

Exemplary step-by-step methods are described schematically in FIG. 14. It will be understood that any portion of the systems and methods disclosed herein can be implemented by computer. Information is collected from the male and female subject regarding his or her fertility associated characteristics 301. This data is then inputted into the central processing unit (CPU) of a computer 302. The CPU is coupled to a storage or memory for storing instructions for implementing methods of the present invention. The instructions, when executed by the CPU, cause the CPU to provide a fertility profile. The CPU provides this determination by inputting the subject data into an algorithm trained on a reference set of data from a plurality of men and women for whom fertility-associated characteristics are known 303. The reference set of data may be stored locally within the computer, such as within the computer memory. Alternatively, the reference set may be stored in a location that is remote from the computer, such as a server. In this instance, the computer communicates across a network to access the reference set of data. The CPU then provides a fertility profile based on the data entered into the algorithm 304.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1—Sample Population for Identification of Infertility-Related Polymorphisms

Genomic DNA is collected from 30 female subjects (15 who have failed multiple rounds of IVF versus 15 who were successful). In particular, all of the subjects are under age 35. Members of the control group succeeded in conceiving through IVF. Members of the test group have a clinical diagnosis of idiopathic infertility, and have failed three of more rounds of IVF with no prior pregnancy. The women are able to produce eggs for IVF and have a reproductively normal male partner. To focus on infertility resulting from oocyte defects (and eliminate factors such as implantation defects) women who have subsequently conceived by egg donation are favored.

### Example 2—Sample Population for Identification of Infertility-Related Polymorphisms

In a follow-up study of a larger cohort, genomic DNA is collected from 300 female subjects (divided into groups having profiles similar to the groups described above). The DNA sequence polymorphisms to be investigated are selected based on the results of small initial studies.

### Example 3—Sample Population for Identification of Premature Ovarian Failure (POF) and Premature Maternal Aging Polymorphisms

Genomic DNA is collected from 30 female subjects who are experiencing symptoms of premature decline in egg quality and reserve including abnormal menstrual cycles or amenorrhea. In particular, all of the subjects are between the ages of 15-40 and have follicle stimulating hormone (FSH) levels of over 20 international units (IU) and a basal antral follicle count of under 5. Members of the control group succeeded in conceiving through IVF. Members of the test group have no previous history of toxic exposure to known fertility damaging treatments such as chemotherapy. Members of this group may also have one or more female family member who experienced menopause before the age of 40.

### Example 4—Sample Procurement and Preparation

Blood is drawn from patients at fertility clinics for standard procedures such as gauging hormone levels and many clinics bank this material after consent for future research projects. Although DNA is easily obtained from blood, wider population sampling is accomplished using home-based, noninvasive methods of DNA collection such as saliva using an Oragene DNA self collection kit (DNA Genotek).

Blood samples—Three-milliliter whole blood samples are venously collected and treated with sodium citrate anticoagulant and stored at 4° C. until DNA extraction.

Whole Saliva—Whole saliva is collected using the Oragene DNA self-collection kit following the manufacturer's instructions. Participants are asked to rub their tongues around the inside of their mouths for about 15 sec and then deposit approximately 2 ml saliva into the collection cup. The collection cup is designed so that the solution from the vial's lower compartment is released and mixes with the saliva when the cap is securely fastened. This starts the initial phase of DNA isolation, and stabilizes the saliva sample for long-term storage at room temperature or in low temperature freezers. Whole saliva samples are stored and shipped, if necessary, at room temperature. Whole saliva has the potential advantage over other non-invasive DNA sampling methods, such as buccal and oral rinse, of providing large numbers of nucleated cells (eg., epithelial cells, leukocytes) per sample.

Blood clots—Clotted blood that is usually discarded after extraction through serum separation, for other laboratory tests such as for monitoring reproductive hormone levels is collected and stored at −80° C. until extraction.

Sample Preparation—Genomic DNA is prepared from patient blood or saliva for downstream sequencing applications with commercially available kits (e.g., Invitrogen.'s ChargeSwitch® gDNA Blood Kit or DNA Genotek kits, respectively). Genomic DNA from clotted is prepared by standard methods involving proteinase K digestion, salt/chloroform extraction and 90% ethanol precipitation of DNA. (see N Kanai et al., 1994, “Rapid and simple method for preparation of genomic DNA from easily obtainable clotted blood,” J Clin Pathol 47:1043-1044, which is incorporated by reference in its entirety for all purposes).

### Example 5—Manufacturing of a Customized Oligonucleotide Library

A customized oligonucleotide library is used to enrich samples for DNAs encoding proteins of interest. Agilent.'s eArray (a web-based design tool) is used to create a customized target enrichment system tailored to infertility related genes. A customized library of 55,000 oligos (120mers) (which covers a 3.3 mb chromosomal region) is designed to target genes of Table 1. The custom RNA oligonucleotides, or baits, are biotinylated for easy capture onto streptavidin-labeled magnetic beads and used in Agilent.'s SureSelect Target Enrichment System.

The target enrichment procedure uses an extremely efficient hybrid selection technique, and significantly improves the cost- and process efficiency of the sequencing workflow. Target sequence enrichment ensures that only the genomic areas of interest can be sequenced, creating process efficiencies that reduce costs and permit more samples to be analyzed per study. The SureSelect Target Enrichment System workflow is solution-based and is performed in microcentrifuge tubes or microtiter plates.

### Example 6—Capture of Genomic DNA

Genomic DNA is sheared and assembled into a library format specific to the sequencing instrument utilized downstream. Size selection is performed on the sheared DNA and confirmed by electrophoresis or other size detection method. The size-selected DNA is incubated with biotinylated RNA oligonucleotides “baits” for 24 hours. The RNA/DNA hybrids are immobilized to streptavidin-labeled magnetic beads, which are captured magnetically. The RNA baits are then digested, leaving only the target selected DNA of interest, which is then amplified and sequenced.

### Example 7—Sequencing of Target Selected DNA

Target-selected DNA is sequenced by a paired end (50 bp) re-sequencing procedure using Illumina.'s Genome Analyzer. The combined DNS targeting and resequencing provides 45 fold redundancy which is greater than the accepted industry standard for SNP discovery.

### Example 8—Correlation of Polymorphisms with Fertility

Polymorphisms among the sequences of target selected DNA from the pool of test subjects are identified, and may be classified according to where they occur in promoters, splice sites, or coding regions of a gene. Polymorphisms can also occur in regions that have no apparent function, such as introns and upstream or downstream non-coding regions. Although such polymorphisms may not be informative as to the functional defect of an allele, nevertheless, they are linked to the defect and useful for predicting infertility (and/or premature ovarian failure (POF), and/or premature maternal aging). The polymorphisms are analyzed statistically to determine their correlation with the fertility status of the test subjects. The statistical analysis indicates that certain polymorphisms identify gene defects that by themselves (homozygous or heterozygous) are sufficient to cause infertility. Other polymorphisms identify genetic variants that reduce, but do not eliminate fertility. Other polymorphisms identify genetic variants that have an apparent effect on fertility only in the presence of particular variants of other genes. Other polymorphisms identify genetic variants that have an apparent effect on fertility only in the presence of particular phenotypes. Other polymorphisms identify genetic variants that have an apparent effect on fertility only in the presence of particular environmental exposures. Still other polymorphisms identify genetic variants that have an apparent effect on fertility only in the presence of any combination of particular variants of other genes, presence of particular phenotypes, and particular environmental exposures.

### Example 9—Diagnostics and Counseling

A library of nucleic acids in an array format is provided for infertility diagnosis. The library consists of selected nucleic acids for enrichment of genetic targets wherein polymorphisms in the targets are correlated with variations in fertility. A patient nucleic acid sample (appropriately cleaved and size selected) is applied to the array, and patient nucleic acids that are not immobilized are washed away. The immobilized nucleic acids of interest are then eluted and sequenced to detect polymorphisms. According to the polymorphisms detected, and in some embodiments, the phenotypic traits and environmental exposures reported, the fertility (or POF or premature maternal aging) status of the patient is evaluated and/or quantified. The patient is accordingly advised as to the suitability and likelihood of success of a fertility treatment or suitability or necessity of a particular in vitro fertilization procedure, whether preventative egg or ovary preservation is indicated, and/or minimization of certain environmental exposures such as alcohol intake or smoking, or mitigation of certain phenotypes such as having children at a younger age is indicated.

### Example 10—Diagnostics and Counseling

A complete DNA sequence of any number of or all of the genes in Table 1 is determined using a targeted resequencing protocol. According to the polymorphisms detected and the phenotypic traits and environmental exposures reported, the fertility status of the patient is evaluated and/or quantified. Alternatively, the POF or maternal aging status of the patient or likelihood of future POF occurrence or premature material aging occurrence is evaluated and/or quantified. The patient is accordingly advised as to the suitability and likelihood of success of a fertility treatment, the suitability or necessity of a particular in vitro fertilization procedure, whether preventative egg or ovary preservation is indicated, and/or minimization of certain environmental exposures such as alcohol intake or smoking, or mitigation of certain phenotypes such as having children at a younger age is indicated.

### Example 11—Whole Genome Sequencing for Female Infertility Biomarker Discovery

The following illustrates use of Whole Genome Sequencing (WGS) data to identify variants of interest in accordance with methods of the invention.

Samples were collected from female patients undergoing fertility treatment at an academic reproductive medical center, and categorized into idiopathic infertility or primary ovarian insufficiency (POI) study groups. Phenotypic information was collected for each patient by mining >200 variables from electronic health records. Genomic DNA extracted from blood samples underwent WGS by Complete Genomics (Mountain View, Calif.). Analysis of genetic variants from WGS was assisted by an infertility knowledgebase with >800 genomic regions of interest (ROI) ranked by a scoring algorithm predicting their likely impact on different fertility disorders, based on publications, data repositories (including protein-protein interactions and tissue expression patterns), meta-analyses of these data, and animal model phenotypes.

The collected female samples were subjected to the processes/algorithms depicted in FIGS. 7-9 (described in more detail above). With those female samples, approximately 50,000 novel variants (approximately 1.6% of total variants observed) were identified as having fertility significances that have not been previously reported in databases such as the sbSNP reference. The identified fertility-related variants included single nucleotide polymorphisms (SNPs, insertions, deletions, copy number variations, inversions, and translocations. Of the SNPs, some of them are predictive to have putative functional significance based on the knowledgebase. For example, the knowledgebase scored some SNPs as deleterious variants due to potential loss of function or changes in protein structure.

In certain aspects, the genomic data, such as WGS data, of a patient/subject population is subjected to a population stratification correction. Population stratification correction accounts for the presence of a systematic difference in allele frequencies between subpopulations in a population possibly due to different ancestry. When conducting population stratification, data is compared to a number (e.g. 1,000) of ethnically diverse individuals as part of the 1000 Genomes Project (100G). Principal components analysis (PCA) is applied to model and identify ancestry differences. In addition, computed association statistics are adjusted for the first two principal components.

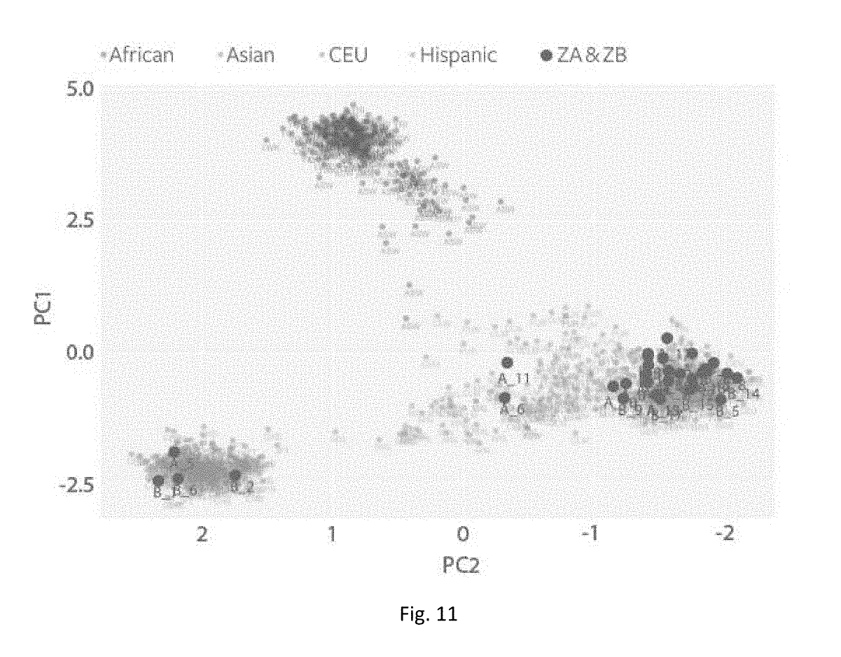

FIG. 11 illustrates population stratification correction of two patient groups. The patient groups include female patients undergoing non-donor in vitro fertilization (IVF) cycles. The patients were 38 years old or younger at the time of enrollment, and had no history of carrying a pregnancy beyond the first term before IVF treatment. Each patient had lack of an apparent cause for infertility (i.e. unexplained) after an evaluation of a complete medical history, physical examination, endocrine profile, and the results of an intimate partner's sperm analysis. The patients were divided into two groups. Group A included 11 patients that experienced no live birth or pregnancy beyond the first trimester after 3 or more IVF cycles. Group B included 18 patients that experienced live birth or pregnancy beyond the first trimester through use of IVF therapy. With population stratification correction. Group A and B patients cluster (are shown as black dots) with East Asian. African. Hispanic, and European individuals as shown in the principal component analysis chart of FIG. 13. This data shows that ethnicity may be linked to infertility, or that certain genomic variations are more prevalent in certain ethnic populations. Accordingly, aspects of the invention involve assessing ethnicity of an individual, either through self-reporting by the individual (e.g., by a questionnaire) or via an assay that looks for known biomarkers related to genetic ethnicity of an individual. That ethnicity data (genetic or self-reported) may be used to guide testing, such as by ensuring that certain genomic variations are checked that are known to be associated with certain ethnic populations.

### Example 12—Cluster Analysis

The following describes specific examples of using the above described cluster analysis to correlate genes not known to be associated with infertility and a known infertility gene.

Activin receptor 2b (ACVR2B) is a significant copy number variation identified in a cohort of patients with infertility (i.e. copy number variation in this gene was identified as being significantly associated with an infertile phenotype in humans). Activin receptor 2B is the receptor bound by Activin, a protein previously known in the art to be involved in both human and mouse reproduction and embryonic development. Activin/Nodal signaling regulates pluripotency and several aspects of patterning during early embryogenesis. Together with Inhibin and Follistatin. Activin is also involved in the complex feedback loops that selectively regulate FSH secretion.

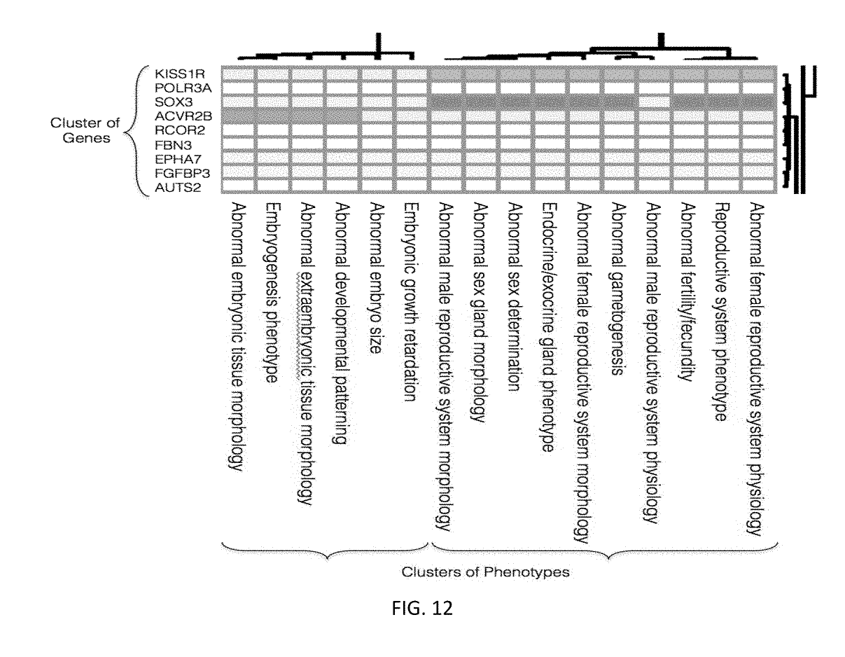

A cluster analysis was performed that compared those features of ACVR2B and features of a plurality of genes not known to be associated with infertility. Based on the cluster analysis, several of the plurality of genes were determined to cluster with the ACVR2B gene due to a commonality between functional and phenotypic features. The genes clustered with the ACVR2B gene were thus identified as potential infertility biomarkers. FIG. 12 illustrates the results of a cluster analysis with ACVR2B.

In yet another example, starting with the known human infertility gene NLRP5, Table 4 lists the most similar (smallest distance) genes to NLRP5. Most of the genes on the list have already been identified based on published studies as having an association with infertility (a validation of the approach), but several have not (e.g., ATAD2B, NR2E1). In this example, ATAD2B, NR2E1 are good candidates for studies/analysis to confirm their infertility association.

TABLE 4

| | | Known | | |
|---|---|---|---|---|
| | | Infertility | | Similarity |
| entrezGeneId | symbol | Association | MouseGeneId | (1-Distance) |
| 126206 | NLRP5 | Y | 23968 | 1 |
| 441161 | OOEP | Y | 67968 | 0.990508 |
| 326340 | ZAR1 | Y | 317755 | 0.954272 |
| 359787 | DPPA3 | Y | 73708 | 0.925278 |
| 54454 | ATAD2B | | 320817 | 0.768295 |
| 8115 | TCL1A | Y | 21432 | 0.729399 |
| 4361 | MRE11A | | 17535 | 0.728909 |
| 4360 | MRC1 | Y | 17533 | 0.727167 |
| 7101 | NR2E1 | | 21907 | 0.719154 |
| 23633 | KPNA6 | | 16650 | 0.712841 |
| 2827 | GPR3 | Y | 14748 | 0.709265 |
| 7783 | ZP2 | Y | 22787 | 0.709177 |
| 200424 | TET3 | | 194388 | 0.707759 |
| 127343 | DMBX1 | Y | 140477 | 0.704141 |
| 10361 | NPM2 | Y | 328440 | 0.700169 |
| 7784 | ZP3 | Y | 22788 | 0.696949 |
| 9210 | BMP15 | Y | 12155 | 0.688272 |
| 22917 | ZP1 | Y | 22786 | 0.688209 |
| 54014 | BRWD1 | Y | 93871 | 0.681323 |
| 344018 | FIGLA | Y | 26910 | 0.674247 |
| 6533 | SLC6A6 | Y | 21366 | 0.673478 |
| 2661 | GDF9 | Y | 14566 | 0.664854 |
| 27252 | KLHL20 | | 226541 | 0.662994 |
| 204801 | NLRP11 | Y | | 0.655971 |
| 654790 | PCP4L1 | Y | 66425 | 0.655923 |

Additionally, starting with a partially characterized gene, CHST8, having incomplete annotation regarding its role in human biological pathways and diseases, including infertility, likely phenotypes/pathways can be imputed based on co-clustered genetic loci. Table 5 shows the genes most similar in function to CHST8 based on the clustering method. The fertility-associated genes FSHB and LHB are characterized as being similar to, or having similar function to CHST8, and are both well characterized independently. Both encode binding proteins for hormones important in female fertility. In this example, CHST8 is therefore a good candidate for studies/analysis to reveal how it is associated with infertility, for example through the disruption of the CHST8 gene in a transgenic mouse model.

TABLE 5

| | | Known | | |
|---|---|---|---|---|
| | | Infertility | | Similarity |
| entrezGeneId | symbol | Association | MouseGeneId | (1-Distance) |
| 64377 | CHST8 | | 68947 | 1 |
| 2488 | FSHB | Y | 14308 | 0.807603 |
| 3972 | LHB | Y | 16866 | 0.799529 |
| 8022 | LHX3 | Y | 16871 | 0.720396 |
| 23373 | CRTC1 | Y | 382056 | 0.68314 |
| 2798 | GNRHR | Y | 14715 | 0.680513 |
| 7425 | VGF | Y | 381677 | 0.673726 |
| 54551 | MAGEL2 | | 27385 | 0.656467 |
| 1813 | DRD2 | Y | 13489 | 0.650742 |
| 5617 | PRL | Y | 19109 | 0.643812 |
| 1081 | CGA | Y | 12640 | 0.62561 |
| 5122 | PCSK1 | | 18548 | 0.624284 |
| 3763 | KCNJ6 | | 16522 | 0.624099 |
| 6447 | SCG5 | Y | 20394 | 0.611227 |
| 6833 | ABCC8 | Y | 20927 | 0.602154 |
| 9985 | REC8 | Y | 56739 | 0.592734 |
| 273 | AMPH | | 218038 | 0.592075 |
| 2688 | GH1 | | 14599 | 0.587602 |
| 4438 | MSH4 | Y | 55993 | 0.571955 |
| 113091 | PTH2 | | 114640 | 0.559548 |
| 11144 | DMC1 | Y | 13404 | 0.55841 |
| 25970 | SH2B1 | Y | 20399 | 0.55654 |
| 6658 | SOX3 | Y | 20675 | 0.553021 |
| 135935 | NOBOX | Y | 18291 | 0.551976 |
| 3990 | LIPC | | 15450 | 0.550449 |

### Example 13—AMH as a Biomarker for Miscarriage, Regardless of Age

Advanced maternal age is a well-established risk factor for pregnancy loss in general and after IVF treatment. Maternal age also associated with lower levels of markers of ovarian reserve, such as AMH. It is less clear, however, how younger patients with abnormal markers of ovarian reserve, should be counseled with respect to the likelihood that a pregnancy will result in a loss.

We performed a retrospective study on patients who achieved pregnancy with IVF at 12 fertility treatment centers in the United States from 2009-2015. Inclusion criteria included patients between the ages of 22-49 in which AMH testing had been performed, having cycles of IVF with both fresh and frozen embryo transfer. Patients with ectopic pregnancies, cycles using donor oocytes or gestational carriers, and cycles where PGS was performed were excluded from this study.

Our analysis included 16,039 IVF cycles (corresponding to 13,463 patients), of which 10,748 cycles resulted in a live birth, 2,733 in a biochemical pregnancy loss (BP), and 2,558 in a clinical miscarriage (CM), which is defined as pregnancy loss after detection of a gestational sac by ultrasound. Time-dependent multivariate time-to-event models were used to evaluate the hazard (risk) of miscarriage for BP and CM when controlling for multiple clinical parameters, such as levels follicle stimulating hormone on Day 3, luteinizing hormone, estradiol, and levels follicle stimulating hormone on Day 3. Predictors were refined using least absolute shrinkage and selection operator (LASSO). As expected, maternal age was confirmed to be a significant risk factor for both BP (4.3% increase/year, P<0.001) and CM (6.9% increase/year, P<0.001).

We next explored the relationship between AMH levels and miscarriage risk. We found that the risk of BP was significantly higher in patients with low AMH, independent of age. Patients with an AMH level of less than 0.2 ng/mL were at a 29.1% higher risk of BP (P=0.01) and patients with an AMH level of between 0.2 ng/ml and 0.95 ng/mL had a 10.4% increased risk (P=0.051).

Surprisingly, we found that a patient's AMH level was a significant predictor of risk of CM in patients with both very low and high AMH levels, independent of their age. Patients with AMH levels of less than 0.2 ng/mL had a 23.8% increased risk (P=0.034), and patients with AMH levels that were greater than 10 ng/mL had a 25.6% increased risk of CM (P=0.02). However, we found that patients' risk of CM was dependent on maternal age if their AMH levels were between 0.2 ng/ml and 0.95 ng/mL; these patients' risk for CM increased by 3.2% per year (P=0.015).

Our study was performed on retrospective data from the United States and future studies may be needed to investigate whether these findings could expand to European practice patterns. Cycles in which PGS was performed were excluded from our study, however including PGS cycles can better resolve the etiology of pregnancy loss in these groups.

This study suggests that AMH is a powerful biomarker for determining risk of miscarriage during IVF treatment. Furthermore, this study suggests that women of any age who have AMH levels less than 0.2 ng/mL or greater than 10 ng/mL have an increased risk of CM. Women who are at risk for miscarriages either due to abnormally low or high AMH level, independent of age, may benefit from increased counseling.

### Example 14—Using Mouse Model Data to Characterize the Genetic Loci Implicated in Human Fertility Potential

Characterization of the genetic basis of human fecundity and fertility disorders permits the development of powerful, rapid, and non-invasive diagnostic tools to help clinicians direct patients to efficient and effective treatment options, as well as in the identification of novel targets for drug development and therapeutics. Moreover, a better understanding of the crucial molecular pathways underlying human fecundity and fertility guides the next generation of targeted, non-hormonal contraceptives.

To this end, association studies in humans and targeted experiments in animal models have contributed to our understanding of the different genetic elements underlying female and male reproductive biology by linking particular genes and genetic variants with various phenotypes of reproduction and fertility, typically on a gene-by-gene basis. Since many knockout mice have similar, if not identical, phenotypes to human patients with lesions in the same/related genetic regions, mouse models represent useful tools with which to model human fecundity and fertility disorders.

In this study, we present data from experiments on mouse models to identify, on a genome-wide scale, genetic loci that are linked to phenotypic characteristics related to reproductive physiology, fecundity, and infertility in both females and males. By defining such relationships in a mammalian model species, and by linking this information to orthologous (and, further, paralogous) genetic loci in humans, we define a powerful data set to be used in the identification and validation of candidate genetic biomarkers of human fecundity, fertility, and infertility and thus identify novel targets for drug development and therapeutics.

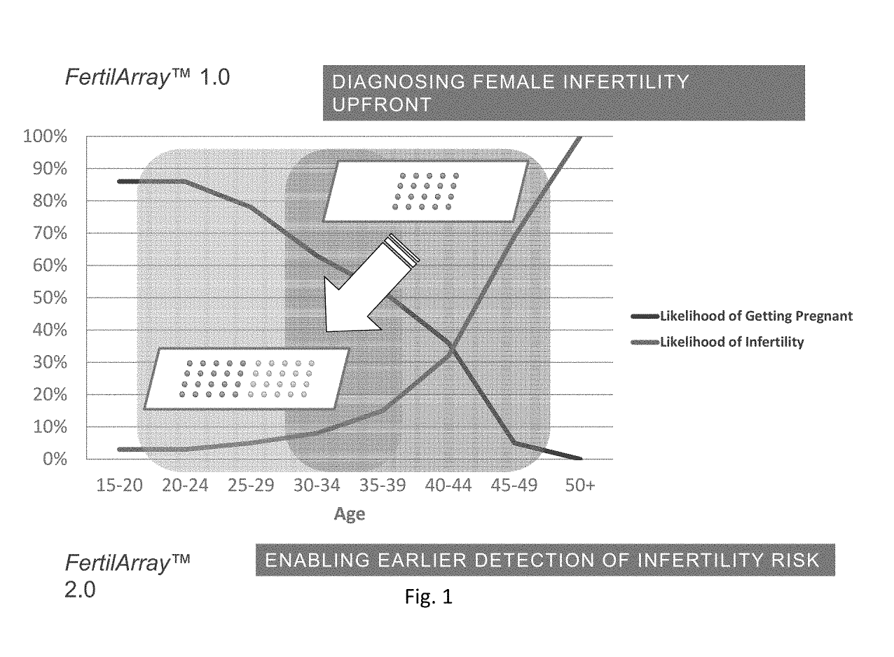

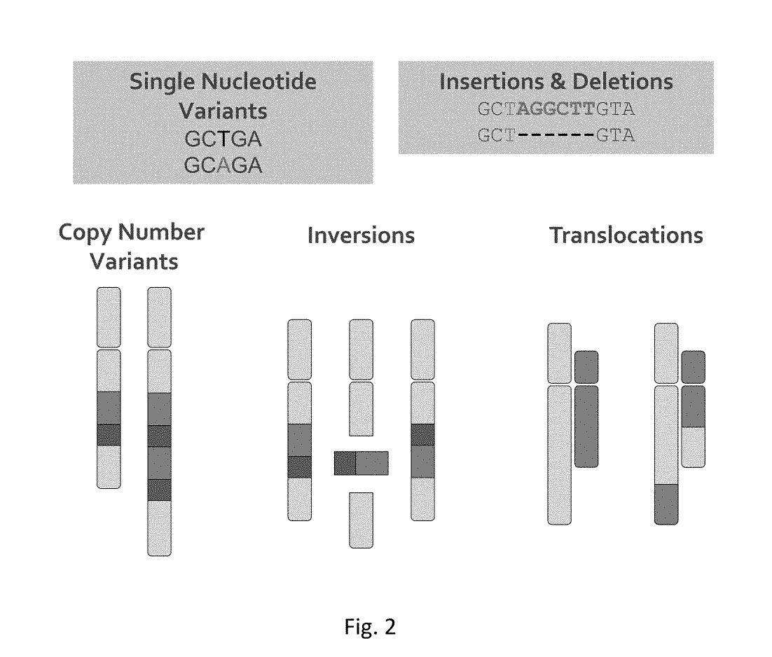

Beginning with the 58,878 mammalian phenotypes described on the international database resource for the laboratory mouse (Mouse Genome Informatics (MGI), http://www.informatics.jax.org/), we narrowed our focus to phenotypes of reproductive physiology, fecundity, and infertility observed when the function of a particular genetic locus is disrupted, as an indicator of whether and how those loci function in particular reproductive processes. As such, we identified 6,045 phenotypes that are either specific to mechanisms of mammalian reproduction or specific to physiological processes that have been directly or indirectly linked to reproduction, fecundity or infertility (Table 6). Next, we further categorized these phenotypes into one or more groups according to the physiological process to which they specifically relate. We numbered these groups from 0 to 21. Certain male and female reproductive phenotypes can be categorized into the same group (i.e. 0, 1, 2, 5, 9, 10, 11) (as shown below). The groups are outlined below:
0. ‘“Infertility” reported’: Phenotypes are assigned to this category if only general descriptions of female or male infertility are made with respect to the mouse model.
1. ‘Gonadogenesis’ encompasses the processes regulating the development of the ovaries and testes, and involves, but is not limited to, primordial germ cell specification and proliferation. Thus, the phenotypes ‘abnormal ovary development’ (MP:0003582) and ‘decreased male germ cell number’ (MP:0004901), among others, are assigned to this category (Fertility category ‘1’, FIGS. 1 and 2).
2. The ‘neuroendocrine axis’ encompasses for example the physiological pathways and structures regulating the production and activity of hormones in a number of different tissues in the human body, including the brain and gonads, hence female-specific phenotypes such as ‘Increased circulating luteinizing hormone level’ (MP:0001751), male-specific phenotypes such as ‘decreased circulating testosterone level’ (MP:0002780) and gender-independent phenotypes such as ‘hypopituitarism’ (MP:0003348), among others, are assigned to this category (Fertility category ‘2’, FIGS. 1 and 2).
3. ‘Folliculogenesis’ encompasses the physiological mechanisms regulating the development of primordial follicles to cystic follicles in the ovary, hence those that are specific to female reproductive biology. The phenotypes ‘Absent cumulus expansion’ (MP:0009374) and ‘Impaired ovarian folliculogenesis’ (MP:0001129), among others, are assigned to this category (Fertility category ‘3’, FIG. 1).
4. ‘Oogenesis’ encompasses the physiological mechanisms regulating the development of primordial oocytes to mature meiosis-II stage oocytes ready to be fertilized, hence those that are specific to female reproductive biology. The phenotypes ‘Abnormal female meiosis’ (MP:0005168) and ‘Oocyte degeneration’ (MP:0009093), among others, are assigned to this category (Fertility category ‘4’, FIG. 1).
5. ‘Oocyte-embryo transition’ encompasses the physiological mechanisms regulating the development of the early embryo and includes mechanisms related to egg quality, such as oocyte cytoplasmic lattice formation, and paternal effect mechanisms. Hence, the phenotypes ‘Inner cell mass degeneration’ (MP:0004965) and ‘paternal effect’ (MP:0010723), among others, are assigned to this category (Fertility category ‘5’, FIG. 1).
6. ‘Placentation (Embryonic)’ encompasses the embryo-specific physiological mechanisms regulating implantation and the development of the placenta. Hence, the phenotypes ‘disorganized extraembryonic tissue’ (MP:0002582) and ‘decreased trophoblast giant cell number’ (MP:0001713), among others, are assigned to this category (Fertility category ‘6’, FIG. 1).
7. ‘Placentation (Uterine)’ encompasses the uterus-specific physiological mechanisms regulating embryo implantation and the development of the placenta. Hence, the phenotypes ‘Abnormal endometrium morphology’ (MP:0004896) and ‘abnormal uterine angiogenesis’ (MP:0009670), among others, are assigned to this category (Fertility category ‘7’, FIG. 1).
8. ‘Post-implantation development’ encompasses the physiological mechanisms regulating post-implantation embryo development, particularly those whose disruption might lead to abnormal development or pregnancy loss in humans. Hence, the phenotypes ‘Failure of primitive streak formation’ (MP:0001693) and ‘Embryonic lethality between implantation and somite formation’ (MP:0006205), among others, are assigned to this category (Fertility category ‘8’, FIG. 1).
9. ‘Adiposity’ encompasses the physiological mechanisms regulating adipose tissue and body weight, which are known to play an important, indirect role in mammalian fecundity and infertility. Hence, the phenotypes ‘Decreased total body fat amount’ (MP:0010025) and ‘Increased adiponectin level’ (MP:0004892), among others, are assigned to this category (Fertility category ‘9’, FIGS. 1 and 2).
10. ‘Reproductive anatomy’ encompasses any phenotype relating to anatomical changes that could impact reproduction, fecundity or fertility. Hence, the phenotypes ‘Vagina atresia’ (MP:0001144) and ‘abnormal seminal vesicle development’ (MP:0013317), among others, are assigned to this category (Fertility category ‘10’, FIGS. 1 and 2).
11. ‘Mouse specific’ encompasses phenotypes of mammalian reproduction that are specific to mice, such as ‘Partial embryonic lethality’ (MP:0011102) or ‘increased litter size” (MP:0001934), among others, are assigned to this category (Fertility category ‘11’, FIGS. 1 and 2), which could relate to analogous processes occurring in other model organisms or indeed humans, such as recurrent pregnancy loss or twinning.
13. ‘Immune response’ encompasses phenotypes that are specific to aspects of immune response mechanisms, which are known to play an important role in mammalian reproduction and fertility. Hence the phenotypes ‘absent uterine NK cells’ (MP:0008047) and ‘Decreased NK T cell number’ (MP:0008040), among others, are assigned to this category (Fertility category ‘13’, FIGS. 1 and 2).
14. ‘Other’ encompasses phenotypes that are known to be associated with changes in fecundity and fertility in humans, or mechanisms that are known to regulate processes specific to these phenotypes. Hence ‘increased cholesterol efflux’ (MP:0003192) and ‘deafness’ (MP:0001967), among others, are assigned to this category (Fertility category ‘14’, FIGS. 1 and 2).
15. ‘Spermatogenesis’ encompasses phenotypes that are specific to processes involved in the production or development of mature spermatozoa, hence those that are specific to male reproductive biology. The phenotypes ‘arrest of spermiogenesis’ (MP:0008279) and ‘oligozoospermia’ (MP:0002687), among others, are assigned to this category (Fertility category ‘15’, FIGS. 2 and 3).
16. ‘Maturation’ encompasses phenotypes that are specific to processes that enable spermatozoa to fertilize eggs, hence those that are specific to male reproductive biology. The phenotypes ‘abnormal spermiation’ (MP:0004182) and ‘abnormal sperm motility’ (MP:0002674) among others, are assigned to this category (Fertility category ‘16’, FIG. 2).
17. ‘Capacitation’ encompasses phenotypes that are specific to functional capacitation of spermatozoa in the vaginal canal and uterus, hence ‘impaired sperm capacitation’ (MP:0003666) among others, are assigned to this category (Fertility category ‘17’, FIG. 2).
18. ‘Fertilization’ encompasses phenotypes relating to the union of a human egg and sperm. Hence, the phenotypes ‘abnormal zona pellucida morphology’ (MP:0003696) and ‘abnormal sperm motility’ (MP:0002674) among others, are assigned to this category (Fertility category ‘18’, FIGS. 15 and 16).
19. ‘Mitosis’ encompasses phenotypes involving changes to the cell division process such that it does not end with two daughter cells that have the same chromosomal complement as the parent cell. Such changes to the mitotic process that may affect for example fertility-related cell proliferation or tissue maintenance, hence ‘abnormal spermatogonia proliferation’ (MP:0002685) and ‘chromosomal breakage’ (MP:0004028), among others, are assigned to this category (Fertility category ‘19’, FIG. 17).
20. ‘Meiosis’ encompasses phenotypes involving changes to the process of meiosis such that it does not result in four daughter cells each with exactly half the chromosome complement of the parent cell, for example during gametogenesis. NB Meiosis could be considered a sub-group of (1) and (15). It includes, among others, phenotypes such as ‘abnormal male meiosis’ (MP:0005169) and ‘meiotic nondisjunction during M1 phase’ (MP:0004218; Fertility category ‘20’, FIG. 17).
21. ‘Spermiogenesis’ encompasses phenotypes involving changes to the morphological differentiation of haploid cells into sperm, hence ‘enlarged sperm head’ (MP:0009233) and ‘elongated sperm flagellum’ (MP:0009240) among others, are assigned to this category (Fertility category ‘21’, FIG. 17).

By correlating reported phenotypes with genetic loci genome-wide, we determined which loci are observed to result in one or more of the 2,493 phenotypes when disrupted in mouse models and assigned those loci into one or more of the numbered categories accordingly (Table 6).

TABLE 6

| | | | Female- | |
|---|---|---|---|---|
| | Male-Specific | | Specific | |
| | Phenotypic | Male Phenotypes Reported | Phenotypic | Female Phenotypes Reported |
| Gene | Category | (PMID) | Category | (PMID) |
| Jund | 15 16 17 10 21 0 | abnormal | 2 | decreased circulating growth |
| | | spermatogenesis (10654608), male | | hormone level (10654608), |
| | | infertility (10654608), | | decreased circulating follicle |
| | | oligozoospermia (10654608), | | stimulating hormone |
| | | teratozoospermia (10654608), | | level (10654608) |
| Pick1 | 15 16 17 1 10 19 20 21 | abnormal | None | |
| | 0 | spermiogenesis (19258705), male | | |
| | | infertility (19258705), | | |
| | | globozoospermia (19258705), | | |
| | | oligozoospermia (19258705), | | |
| | | teratozoospermia (19258705), | | |
| | | abnormal sperm flagellum | | |
| | | morphology (19258705), abnormal | | |
| | | acrosome morphology (19258705), | | |
| | | abnormal sperm nucleus | | |
| | | morphology (19258705), | | |
| Npc1 | 15 18 10 21 0 9 | arrest of | 9 | decreased susceptibility to diet- |
| | | spermatogenesis (16850391), male | | induced obesity (15671032) |
| | | infertility (16850391), | | |
| | | oligozoospermia (16850391), | | |
| | | teratozoospermia (16850391), | | |
| | | absent sperm flagellum (16850391), | | |
| | | abnormal sperm head | | |
| | | morphology (16850391), absent | | |
| | | sperm head (16850391), | | |
| Camk4 | 15 1 10 21 0 | abnormal | 0 3 4 10 11 | reduced female |
| | | spermiogenesis (10932193), arrest | 14 | fertility (11108293)polyovular |
| | | of spermatogenesis (10932193), | | ovarian follicle (11108293), |
| | | male infertility (10932193), | | absent corpus luteum (11108293), |
| | | oligozoospermia (10932193), | | abnormal ovarian follicle |
| | | teratozoospermia (10932193), | | morphology (11108293) abnormal |
| | | decreased male germ cell | | gametogenesis (10932193), |
| | | number (10932193), abnormal | | anovulation (11108293) abnormal |
| | | spermatid morphology (10932193), | | female reproductive system |
| | | abnormal sperm flagellum | | morphology (11108293)decreased |
| | | morphology (10932193), abnormal | | litter size (11108293)premature |
| | | sperm head morphology (10932193), | | death (11108293), decreased body |
| | | | | weight (10932193, 20209163) |
| Tbc1d20 | 15 16 17 1 10 21 0 | abnormal spermiogenesis (3955134), | None | |
| | | arrest of spermatogenesis (6863898), | | |
| | | male infertility (3955134, 6863898), | | |
| | | oligozoospermia (6863898, 3955134), | | |
| | | teratozoospermia (3955134), | | |
| | | abnormal male germ cell | | |
| | | morphology (3955134), absent | | |
| | | acrosome (3955134), abnormal | | |
| | | sperm head morphology (3955134), | | |
| | | arrest of | | |
| | | spermatogenesis (14757819), | | |
| | | decreased male germ cell | | |
| | | number (24239381), absent | | |
| | | acrosome (24239381), | | |
| Rxrb | 15 16 17 10 21 0 | arrest of spermatogenesis (8557197), | 114 | decreased germ cell |
| | | male infertility (8557197), | | number (8557197)partial perinatal |
| | | oligozoospermia (8557197), | | lethality (8557197), decreased |
| | | teratozoospermia (8557197), absent | | cholesterol efflux (14993927), |
| | | acrosome (8557197), abnormal | | partial prenatal lethality (8557197) |
| | | acrosome morphology (8557197), | | |
| | | detached acrosome (8557197), | | |
| | | coiled sperm flagellum (8557197), | | |
| | | abnormal sperm mitochondrial | | |
| | | sheath morphology (8557197), | | |
| Cadm1 | 15 16 17 1 10 21 0 | arrest of | 14 | decreased body |
| | | spermatogenesis (16611999), male | | weight (22084409) |
| | | infertility (16611999), | | |
| | | oligozoospermia (16611999), | | |
| | | teratozoospermia (16611999), arrest | | |
| | | of spermatogenesis (16612000), | | |
| | | male infertility (16612000), | | |
| | | globozoospermia (16612000), | | |
| | | oligozoospermia (16612000), | | |
| | | teratozoospermia (16612000), short | | |
| | | sperm flagellum (16612000), | | |
| | | multiflagellated sperm (16612000), | | |
| | | male infertility (16382161), | | |
| | | oligozoospermia (16382161), | | |
| | | teratozoospermia (16382161), | | |
| | | decreased male germ cell | | |
| | | number (16382161), abnormal | | |
| | | spermatid morphology (16382161), | | |
| | | arrest of spermiogenesis (16382161), | | |
| | | abnormal spermatid | | |
| | | morphology (18055550), | | |
| Sirt1 | 15 16 17 1 10 20 | globozoospermia (12482959), | 0 1 2 3 4 5 8 | female infertility (17877786), |
| | 21 0 9 | abnormal spermatocyte | 9 10 11 13 14 | reduced female |
| | | morphology (18987333), | | fertility (12482959)small |
| | | teratozoospermia (12482959), | | ovary (12482959)decreased |
| | | oligozoospermia (12960381, 12482959, | | circulating luteinizing hormone |
| | | 22006156), abnormal | | level (18987333), absent estrous |
| | | spermatogenesis (22006156), male | | cycle (12482959), decreased |
| | | infertility (12482959, 18987333, 17877786, | | circulating follicle stimulating |
| | | 22006156), abnormal Sertoli | | hormone level (18987333), |
| | | cell development (18987333), | | abnormal estrous |
| | | abnormal spermatid | | cycle (12482959), decreased |
| | | morphology (12482959, 18987333), | | circulating thyroxine |
| | | abnormal sperm flagellum | | level (18335035)absent corpus |
| | | morphology (12482959), arrest of | | luteum (12482959) abnormal cell |
| | | spermatogenesis (18987333), arrest | | cycle checkpoint |
| | | of male meiosis (18987333) | | function (18835033), abnormal |
| | | | | DNA repair (18835033), |
| | | | | anovulation (12482959) abnormal |
| | | | | cell cycle checkpoint |
| | | | | function (18835033), increased |
| | | | | mitotic index (18835033) abnormal |
| | | | | cell cycle checkpoint |
| | | | | function (18835033), Xabsent |
| | | | | limb buds (18835033), increased |
| | | | | mitotic index (18835033), |
| | | | | *decreased embryo |
| | | | | size (12960381, 12482959, 18835033) |
| | | | | abnormal inguinal fat pad |
| | | | | morphology (18335035), |
| | | | | decreased white adipose tissue |
| | | | | amount (22006156)Xabnormal |
| | | | | mammary gland growth during |
| | | | | pregnancy (17201918), abnormal |
| | | | | uterus size (12482959)partial |
| | | | | embryonic lethality during |
| | | | | organogenesis (18835033), partial |
| | | | | lethality throughout fetal growth |
| | | | | and |
| | | | | development (18835033)*decreased |
| | | | | CD8-positive T cell |
| | | | | number (12482959)*decreased |
| | | | | body |
| | | | | size (12960381, 18987333, 17877786, |
| | | | | 22006156, 20668205), *slow |
| | | | | postnatal weight gain (18335035), |
| | | | | partial postnatal |
| | | | | lethality (12960381, 22006156), |
| | | | | complete postnatal |
| | | | | lethality (12482959), partial |
| | | | | perinatal |
| | | | | lethality (12960381, 17201918, 12482959), |
| | | | | decreased survivor |
| | | | | rate (18835033), premature |
| | | | | death (18987333), postnatal |
| | | | | lethality (17201918), partial |
| | | | | neonatal lethality (17201918), |
| | | | | decreased body |
| | | | | weight (17201918, 12482959, 17877786) |
| Meig1 | 15 16 17 1 21 0 | arrest of | None | |
| | | spermatogenesis (19805151), male | | |
| | | infertility (19805151), | | |
| | | azoospermia (19805151), absent | | |
| | | sperm flagellum (19805151), | | |
| | | abnormal sperm head | | |
| | | morphology (19805151), abnormal | | |
| | | sperm axoneme | | |
| | | morphology (19805151), abnormal | | |
| | | spermatogenesis (20004656), male | | |
| | | infertility (20004656), | | |
| | | oligozoospermia (20004656), | | |
| | | teratozoospermia (20004656), | | |
| | | abnormal sperm flagellum | | |
| | | morphology (20004656), abnormal | | |
| | | sperm head morphology (20004656), | | |
| | | enlarged sperm head (20004656), | | |
| Tcte3 | 15 16 17 1 10 21 0 | arrest of | None | |
| | | spermatogenesis (19778998), male | | |
| | | infertility (19778998), | | |
| | | oligozoospermia (19778998), | | |
| | | abnormal sperm flagellum | | |
| | | morphology (19778998), abnormal | | |
| | | sperm head morphology (19778998), | | |
| | | multiflagellated sperm (19778998), | | |
| Ak7 | 15 21 0 | azoospermia (18776131), | None | |
| | | oligozoospermia (21746835), male | | |
| | | infertility (21746835, 18776131), | | |
| | | arrest of | | |
| | | spermatogenesis (21746835), | | |
| | | abnormal sperm head | | |
| | | morphology (18776131) | | |
| Akap9 | 15 1 10 20 21 0 9 | male infertility (12855593), arrest | 9 | decreased percent body fat ( ), |
| | | of male meiosis (12855593), male | | decreased total body fat amount ( ) |
| | | infertility ( ), globozoospermia ( ), | | |
| | | oligozoospermia ( ), male | | |
| | | infertility (23608191), abnormal | | |
| | | spermatogenesis (23608191), | | |
| | | azoospermia (23608191), abnormal | | |
| | | Sertoli cell | | |
| | | development (23608191), abnormal | | |
| | | spermatocyte | | |
| | | morphology (23608191), arrest of | | |
| | | spermatogenesis (23608191), | | |
| Zbtb16 | 15 16 17 1 10 19 | abnormal | 4, 5, 8 | abnormal DNA |
| | 21 2 0 | spermatogenesis (15156143), | | methylation (23727884) abnormal |
| | | oligozoospermia (15156143), | | DNA methylation (23727884), |
| | | abnormal spermatogonia | | abnormal epigenetic regulation of |
| | | proliferation (15156143), increased | | gene |
| | | circulating testosterone | | expression (23727884) abnormal |
| | | level (15156143), abnormal | | epigenetic regulation of gene |
| | | spermatogonia | | expression (23727884) |
| | | morphology (15156143), | | |
| | | azoospermia (15156142), decreased | | |
| | | male germ cell number (15156142), | | |
| | | male infertility (5088020), | | |
| | | azoospermia (5088020), male | | |
| | | infertility (6067640), reduced male | | |
| | | fertility ( ), | | |
| Cyp19a1 | 15 16 17 1 10 21 | arrest of spermiogenesis (10393934), | 0 1 2 3 4 7 9 | female |
| | 2 0 9 | decreased male germ cell | 10 13 14 | infertility (9618522, 10875266, 10862797, |
| | | number (10393934), | | 11431142, 11241177, 9826549) |
| | | oligozoospermia (11545296), | | abnormal ovary |
| | | abnormal | | morphology (9618522, 10875266, 11431142, |
| | | spermatogenesis (10393934, 11545296), | | 9826549, 12205030)increased |
| | | abnormal acrosome | | circulating leptin |
| | | morphology (10393934), increased | | level (11070087), increased |
| | | circulating testosterone | | circulating follicle stimulating |
| | | level (11356695, 10393934, 9618522, | | hormone |
| | | 11241177, 11431142, 11162635, 12553872), | | level (10875266, 10393934, 9618522), |
| | | abnormal male germ cell | | increased circulating |
| | | morphology (10393934), male | | dihydrotestosterone |
| | | infertility (10393934, 12845227), | | level (11356695), increased |
| | | abnormal | | circulating luteinizing hormone |
| | | spermiogenesis (10393934), | | level (9618522, 10875266, 10393934), |
| | | abnormal spermatid | | decreased circulating |
| | | morphology (10393934), reduced | | estradiol |
| | | male | | level (11431142, 11162635), |
| | | fertility (11241177, 9826549, 11545296, | | increased circulating prolactin |
| | | 12845227) | | level (11356695)absent corpus |
| | | | | luteum (9618522, 10875266, 11431142, |
| | | | | 9826549, 12205030), |
| | | | | abnormal mature ovarian follicle |
| | | | | morphology (12205030), |
| | | | | abnormal granulosa cell |
| | | | | morphology (10875266), impaired |
| | | | | ovarian |
| | | | | folliculogenesis (9618522, 10875266, |
| | | | | 12205030), abnormal ovarian |
| | | | | follicle morphology (12205030), |
| | | | | absent ovarian |
| | | | | follicles (11431142), impaired |
| | | | | luteinization (11431142)anovulation |
| | | | | (10875266, 11431142)thin |
| | | | | endometrium (11431142)increased |
| | | | | total body fat |
| | | | | amount (9826549, 11070087), |
| | | | | increased fat cell size (11070087), |
| | | | | increased renal fat pad |
| | | | | weight (12553872, 11070087), |
| | | | | increased mammary fat pad |
| | | | | weight (9618522), increased |
| | | | | gonadal fat pad |
| | | | | weight (9618522, 12553872, 11070087), |
| | | | | increased abdominal fat pad |
| | | | | weight (10862797, 12553872)Xenlarged |
| | | | | clitoris (9618522), small |
| | | | | uterus (11431142), decreased |
| | | | | uterus |
| | | | | weight (10875266, 11431142, 11162635, |
| | | | | 9826549), thin |
| | | | | myometrium (11431142), |
| | | | | abnormal uterus |
| | | | | development (10875266, 9826549), |
| | | | | ovary |
| | | | | hemorrhage (10875266, 11431142, |
| | | | | 12205030), uterus |
| | | | | hypoplasia (9618522), ovary |
| | | | | cysts (10875266, 11431142, 12205030), |
| | | | | Xabnormal labium |
| | | | | morphology (9618522)*increased |
| | | | | osteoclast cell |
| | | | | number (11162635)obese (12553872), |
| | | | | increased susceptibility to |
| | | | | weight gain (12553872), abnormal |
| | | | | auditory brainstem |
| | | | | response (18317592), increased |
| | | | | body |
| | | | | weight (12553872, 11070087) |
| Gmcl1 | 15 16 17 19 20 21 0 | abnormal | 11 | decreased litter size (12556490) |
| | | spermatogenesis (12556490), | | |
| | | abnormal | | |
| | | spermiogenesis (12556490), | | |
| | | reduced male fertility (12556490), | | |
| | | globozoospermia (12556490), | | |
| | | oligozoospermia (12556490), | | |
| | | teratozoospermia (12556490), | | |
| | | abnormal spermiation (12556490), | | |
| | | abnormal spermatocyte | | |
| | | morphology (12556490), absent | | |
| | | sperm flagellum (12556490), | | |
| | | abnormal sperm flagellum | | |
| | | morphology (12556490), abnormal | | |
| | | acrosome morphology (12556490), | | |
| | | abnormal sperm head | | |
| | | morphology (12556490), abnormal | | |
| | | sperm nucleus | | |
| | | morphology (12556490), | | |
| Ube2b | 15 16 17 1 10 20 | abnormal | 4, 5 | abnormal chiasmata |
| | 21 0 | spermatogenesis (12556476), male | | formation (12556476), abnormal |
| | | infertility (12556476), abnormal | | double-strand DNA break |
| | | male meiosis (12556476), abnormal | | repair (21807948) abnormal |
| | | spermatocyte | | double-strand DNA break |
| | | morphology (12556476), abnormal | | repair (21807948) |
| | | spermatogenesis (8797826), male | | |
| | | infertility (8797826), | | |
| | | oligozoospermia (8797826), | | |
| | | teratozoospermia (8797826), | | |
| | | abnormal spermatocyte | | |
| | | morphology (8797826), abnormal | | |
| | | sperm head morphology (8797826), | | |
| | | abnormal sperm midpiece | | |
| | | morphology (8797826), | | |
| Egr4 | 15 1 10 20 21 0 | detached sperm | None | |
| | | flagellum (10529423), decreased | | |
| | | male germ cell number (10529423), | | |
| | | teratozoospermia (10529423), | | |
| | | kinked sperm flagellum (10529423), | | |
| | | oligozoospermia (10529423), | | |
| | | abnormal male meiosis (10529423), | | |
| | | abnormal | | |
| | | spermatogenesis (10529423), coiled | | |
| | | sperm flagellum (10529423), male | | |
| | | infertility (10529423), abnormal | | |
| | | sperm flagellum | | |
| | | morphology (10529423), abnormal | | |
| | | sperm head morphology (10529423) | | |
| Esr1 | 15 16 17 18 1 10 | abnormal | 0 1 2 3 4 5 7 | infertility (19188600), abnormal |
| | 21 2 0 9 | spermatogenesis (8895349), | 9 10 11 13 14 | female reproductive system |
| | | decreased circulating testosterone | | physiology (8248223), female |
| | | level (17495854), increased | | infertility (8248223, 18339713, 10976058, |
| | | circulating testosterone | | 19574448, 22800760) abnormal |
| | | level (8895349, 17495854, 18339713, | | ovary |
| | | 21444817, 20667977), detached | | morphology (8248223, 10919287, 10976058), |
| | | sperm flagellum (8895349), | | abnormal mesonephros |
| | | decreased male germ cell | | morphology (11014235)increased |
| | | number (10976058), | | circulating luteinizing hormone |
| | | teratozoospermia (8895349), male | | level (18339713, 21444817, 14583652, |
| | | infertility (8895349, 11698654, 22800760), | | 20667977), increased |
| | | increased epididymal fat pad | | circulating leptin |
| | | weight (11070086), reduced male | | level (11095962), decreased |
| | | fertility (8248223), | | lactotroph cell number (9171231), |
| | | oligozoospermia (8248223, 8895349, | | *decreased circulating insulin- |
| | | 10670526, 18755802) | | like growth factor I |
| | | | | level (10805804, 10558910), |
| | | | | decreased circulating prolactin |
| | | | | level (10919287), absent estrous |
| | | | | cycle (10976058, 21873215), |
| | | | | decreased circulating estradiol |
| | | | | level (17495854), increased |
| | | | | circulating estradiol |
| | | | | level (8584021, 11784006, 18339713, |
| | | | | 21444817, 12855748, 21873215), |
| | | | | abnormal pituitary gland |
| | | | | physiology (9171231)absent |
| | | | | corpus |
| | | | | luteum (8248223, 10919287, 10342864, |
| | | | | 18339713, 10976058, 21873215, |
| | | | | 22800760), impaired ovarian |
| | | | | folliculogenesis (10342864), |
| | | | | decreased primordial ovarian |
| | | | | follicle number (21873215), |
| | | | | abnormal ovarian follicle |
| | | | | morphology (10976058, 21873215), |
| | | | | absent mature ovarian |
| | | | | follicles (22800760), abnormal |
| | | | | secondary ovarian follicle |
| | | | | morphology (18339713), |
| | | | | increased thecal cell |
| | | | | number (18339713), impaired |
| | | | | granulosa cell |
| | | | | differentiation (18339713), |
| | | | | abnormal ovarian |
| | | | | folliculogenesis (10976058)impaired |
| | | | | fertilization (8895349), |
| | | | | anovulation (10919287, 18339713) |
| | | | | impaired |
| | | | | fertilization (8895349)*abnormal |
| | | | | vascular wound |
| | | | | healing (20577047), failure of |
| | | | | embryo implantation (12297545), |
| | | | | abnormal endometrium |
| | | | | morphology (11311804), |
| | | | | decreased endometrial gland |
| | | | | number (21873215)increased |
| | | | | inguinal fat pad |
| | | | | weight (11070086), increased total |
| | | | | body fat |
| | | | | amount (11095962, 20667977), |
| | | | | increased renal fat pad |
| | | | | weight (11070086), increased |
| | | | | mammary fat pad |
| | | | | weight (18339713), increased |
| | | | | gonadal fat pad |
| | | | | weight (11095962, 18339713), |
| | | | | increased parametrial fat pad |
| | | | | weight (11070086), increased |
| | | | | white fat cell number (11070086), |
| | | | | increased white fat cell |
| | | | | size (11070086), increased |
| | | | | retroperitoneal fat pad |
| | | | | weight (11095962), increased |
| | | | | white adipose tissue |
| | | | | amount (11070086) abnormal |
| | | | | uterus development (20667977), |
| | | | | *abnormal vagina |
| | | | | morphology (18339713), ovary |
| | | | | hemorrhage (10919287, 10342864, |
| | | | | 19574448, 22800760), *abnormal |
| | | | | vagina epithelium |
| | | | | morphology (11784006), small |
| | | | | uterus (11311804, 18339713, 19574448, |
| | | | | 21873215), uterus |
| | | | | hypoplasia (8248223, 11784006, 18339713, |
| | | | | 10976058, 21873215, 22800760), |
| | | | | *vagina |
| | | | | hypoplasia (18339713, 10976058), |
| | | | | ovary |
| | | | | cysts (18339713, 10976058, 22800760), |
| | | | | decreased uterus |
| | | | | weight (10558910, 11784006, 17495854, |
| | | | | 16234973), ovarian |
| | | | | follicular |
| | | | | cyst (8248223, 10919287, 10342864, |
| | | | | 11784006), abnormal uterus |
| | | | | morphology (11311804, 18339713), |
| | | | | uterus |
| | | | | cysts (21873215) abnormal |
| | | | | superovulation (10342864)*increased |
| | | | | plasma cell |
| | | | | number (14745006), *abnormal |
| | | | | class switch |
| | | | | recombination (12603601), |
| | | | | *abnormal |
| | | | | hematopoiesis (10875230), |
| | | | | *decreased immature B cell |
| | | | | number (9647203, 10875230), |
| | | | | *decreased CD4-positive T cell |
| | | | | number (11380688), *abnormal B |
| | | | | cell number (12603601), |
| | | | | *decreased thymocyte |
| | | | | number (10510352), *decreased |
| | | | | mature B cell |
| | | | | number (9647203, 10875230), |
| | | | | *decreased CD8-positive T cell |
| | | | | number (11380688), *decreased B |
| | | | | cell number (10875230), |
| | | | | *increased osteoclast cell |
| | | | | number (21444817), *increased |
| | | | | double-positive T cell |
| | | | | number (11380688), *abnormal B |
| | | | | cell |
| | | | | differentiation (10875230)*abnormal |
| | | | | nociception after |
| | | | | inflammation (19285805), |
| | | | | *increased body size (18339713), |
| | | | | obese (11095962, 11593044, 22800760), |
| | | | | *hyporesponsive to tactile |
| | | | | stimuli (19285805), increased |
| | | | | body |
| | | | | weight (10558910, 11784006, 17495854, |
| | | | | 18339713, 11070086, 22800760, |
| | | | | 20667977), *decreased body |
| | | | | length (14753739), decreased |
| | | | | body weight (10805804) |
| Fshb | 15 16 17 1 10 21 | decreased male germ cell | 0 1 2 3 10 | |
| | | number (11416011), | | |
| | | oligozoospermia (9020850), | | |
| | | abnormal | | |
| | | spermatogenesis (11416011), | | |
| | | abnormal spermatogonia | | |
| | | morphology (11416011), abnormal | | |
| | | spermatid morphology (11416011) | | |
| Rara | 15 1 10 21 0 | abnormal | None | |
| | | spermatogenesis (8394014), male | | |
| | | infertility (8394014), | | |
| | | oligozoospermia (8394014), male | | |
| | | infertility (11857786), abnormal | | |
| | | spermiogenesis (15901285), male | | |
| | | infertility (15901285), | | |
| | | oligozoospermia (15901285), | | |
| | | teratozoospermia (15901285), | | |
| | | abnormal spermatid | | |
| | | morphology (15901285), | | |
| Fshr | 15 16 17 1 10 19 | decreased circulating testosterone | 0 1 2 3 4 7 9 | |
| | 20 21 2 0 9 | level (9811848, 10775161, 11466221), | 10 11 13 14 | |
| | | abnormal testis | | |
| | | development (11466221), abnormal | | |
| | | sperm axoneme | | |
| | | morphology (15973687), delayed | | |
| | | male fertility (11466221), Sertoli | | |
| | | cell hypoplasia (11466221), | | |
| | | teratozoospermia (15973687), | | |
| | | abnormal spermatogonia | | |
| | | proliferation (11466221), kinked | | |
| | | sperm flagellum (10775161), | | |
| | | oligozoospermia (9811848, 15973687), | | |
| | | abnormal | | |
| | | spermatogenesis (9811848, 10775161), | | |
| | | increased circulating testosterone | | |
| | | level (11089565, 11459817), | | |
| | | enlarged sperm head (10775161), | | |
| | | abnormal sperm nucleus | | |
| | | morphology (15973687), abnormal | | |
| | | spermatogonia | | |
| | | morphology (10775161), detached | | |
| | | acrosome (15973687), abnormal | | |
| | | spermiogenesis (10775161, 11466221), | | |
| | | abnormal spermatid | | |
| | | morphology (10775161, 11466221), | | |
| | | abnormal sperm flagellum | | |
| | | morphology (9811848, 10775161), | | |
| | | reduced male | | |
| | | fertility (9811848, 10775161, 11466221), | | |
| | | abnormal sperm head | | |
| | | morphology (15973687) | | |
| Npepps | 15 16 17 10 21 2 0 | abnormal | 0 2 11 | reduced |
| | | spermatogenesis (11376114), male | | fertility (18209067)decreased |
| | | infertility (11376114), | | circulating luteinizing hormone |
| | | oligozoospermia (11376114), | | level (11376114), decreased |
| | | decreased circulating testosterone | | circulating prolactin |
| | | level (11376114), abnormal | | level (11376114), decreased |
| | | spermatid morphology (11376114), | | circulating follicle stimulating |
| | | | | hormone |
| | | | | level (11376114)decreased litter |
| | | | | size (18209067) |
| Hip1 | 15 1 10 21 0 | reduced male fertility (14998932), | None | |
| | | abnormal spermatid | | |
| | | morphology (14998932), abnormal | | |
| | | spermatogenesis (11604514), | | |
| | | oligozoospermia (11604514), | | |
| | | decreased male germ cell | | |
| | | number (11604514), male | | |
| | | infertility (14998932), | | |
| Golga3 | 15 16 17 18 1 10 | abnormal | 4 5 | decreased fertilization |
| | 21 0 | spermatogenesis (23495255), | | frequency (23495255), impaired |
| | | abnormal | | fertilization (23495255)decreased |
| | | spermiogenesis (23495255), male | | fertilization |
| | | infertility (23495255), | | frequency (23495255), impaired |
| | | globozoospermia ( ), | | fertilization (23495255) |
| | | oligozoospermia ( ), | | |
| | | azoospermia (23495255), | | |
| | | teratozoospermia (23495255), | | |
| | | decreased male germ cell | | |
| | | number (23495255), absent sperm | | |
| | | flagellum ( ), detached sperm | | |
| | | flagellum (23495255), abnormal | | |
| | | sperm head morphology (23495255), | | |
| | | male infertility (9892724), | | |
| | | oligozoospermia (9892724), | | |
| | | abnormal sperm head | | |
| | | morphology (9892724), | | |
| Tssk6 | 15 16 17 21 0 | abnormal | None | |
| | | spermatogenesis (15870294), male | | |
| | | infertility (15870294), | | |
| | | oligozoospermia (15870294), | | |
| | | abnormal sperm head | | |
| | | morphology (15870294), | | |
| Etv5 | 15 16 17 1 10 19 | azoospermia (16107850, 24204802), | 0 8 11 14 | female |
| | 20 21 0 | abnormal spermatocyte | | infertility (24204802)complete |
| | | morphology (16107850), decreased | | embryonic |
| | | male germ cell number (24204802), | | lethality (19898483)partial |
| | | abnormal spermatogonia | | lethality throughout fetal growth |
| | | proliferation (16107850), | | and |
| | | oligozoospermia (18032421), | | development (24204802)partial |
| | | abnormal | | postnatal lethality (24204802), |
| | | spermatogenesis (16107850), | | decreased body |
| | | abnormal spermatogonia | | weight (18032421, 24204802) |
| | | morphology (16107850, 18032421), | | |
| | | abnormal spermiation (18032421), | | |
| | | male | | |
| | | infertility (18032421, 16107850, 24204802) | | |
| Kcnj6 | 15 16 17 1 10 21 0 | abnormal | 9 | decreased subcutaneous adipose |
| | | spermatogenesis (7760215), | | tissue amount (20074528), |
| | | globozoospermia (7760215), | | decreased abdominal fat pad |
| | | oligozoospermia (7760215), | | weight (20074528) |
| | | abnormal male germ cell | | |
| | | morphology (7760215), abnormal | | |
| | | spermatid morphology (10766925), | | |
| | | male infertility (8081012), | | |
| | | azoospermia (8081012), reduced | | |
| | | male fertility ( ), | | |
| Qk | 15 16 21 0 | abnormal | 5 7 8 11 | abnormal embryogenesis/ |
| | | spermatogenesis (14757819), | | development (3410318) abnormal |
| | | abnormal | | visceral yolk sac |
| | | spermiogenesis (14757819), | | morphology (14706070, 16470614), |
| | | oligozoospermia (14757819), | | *abnormal |
| | | abnormal sperm flagellum | | vasculogenesis (11892011), |
| | | morphology (14757819), abnormal | | abnormal vitelline vasculature |
| | | sperm head morphology (14757819), | | morphology (16470614, 11892011, |
| | | necrospermia (14757819), reduced | | 16470614)complete embryonic |
| | | male fertility (14169723), | | lethality between somite |
| | | | | formation and embryo |
| | | | | turning (3410318), *embryonic |
| | | | | growth retardation (16470614), |
| | | | | abnormal neural tube |
| | | | | morphology/development (14706070), |
| | | | | complete embryonic lethality |
| | | | | during |
| | | | | organogenesis (14706070, 11892011, |
| | | | | 16470614, 16470614), Xabsent |
| | | | | somites (3410318), abnormal |
| | | | | developmental |
| | | | | patterning (3410318), embryonic |
| | | | | growth arrest (3410318), abnormal |
| | | | | embryogenesis/ |
| | | | | development (3410318), |
| | | | | *decreased embryo |
| | | | | size (14706070, 3410318), Xwavy |
| | | | | neural tube (14706070), Xopen |
| | | | | neural tube (14706070), abnormal |
| | | | | neural plate |
| | | | | morphology (3410318), abnormal |
| | | | | anterior visceral endoderm |
| | | | | morphology (16470614) |
| H1fnt | 15 16 17 18 19 20 | abnormal | 5 | impaired |
| | 21 0 | spermiogenesis (15710904), | | fertilization (16055721)impaired |
| | | reduced male fertility (15710904), | | fertilization (16055721) |
| | | oligozoospermia (15710904), | | |
| | | abnormal sperm head | | |
| | | morphology (15710904), detached | | |
| | | acrosome (15710904), abnormal | | |
| | | sperm nucleus | | |
| | | morphology (15710904), abnormal | | |
| | | spermatogenesis (16055721), male | | |
| | | infertility (16055721), | | |
| | | teratozoospermia (16055721), | | |
| Kdm3a | 15 16 17 1 10 21 | teratozoospermia (17943087), | 2 9 14 | increased circulating leptin |
| | 0 9 | increased epididymal fat pad | | level (19624751) abnormal adipose |
| | | weight (19624751), | | tissue amount (19194461), |
| | | oligozoospermia (17943087, 19910458), | | increased brown adipose tissue |
| | | abnormal | | amount (19194461), increased fat |
| | | spermatogenesis (17943087, 19194461, | | cell size (19624751), abnormal |
| | | 19910458), male | | brown adipose tissue |
| | | infertility (17943087, 19910458), | | morphology (19194461), |
| | | abnormal | | abnormal white adipose tissue |
| | | spermiogenesis (17943087, 19910458), | | morphology (19624751), |
| | | abnormal spermatid | | abnormal brown adipose tissue |
| | | morphology (17943087, 19910458) | | physiology (19194461), increased |
| | | | | retroperitoneal fat pad |
| | | | | weight (19624751), increased |
| | | | | white adipose tissue |
| | | | | amount (19624751), increased |
| | | | | white fat cell lipid droplet |
| | | | | size (19194461)obese (19194461, 19624751), |
| | | | | increased susceptibility |
| | | | | to weight gain (19624751), |
| | | | | increased susceptibility to diet- |
| | | | | induced obesity (19194461) |
| Ehd4 | 15 16 1 10 21 0 | abnormal | None | |
| | | spermatogenesis (20213691), | | |
| | | reduced male fertility (20213691), | | |
| | | oligozoospermia (20213691), | | |
| | | abnormal spermiation (20213691), | | |
| | | decreased male germ cell | | |
| | | number (20213691), abnormal | | |
| | | spermatid morphology (20213691), | | |
| Taf4b | 15 16 17 1 10 19 | decreased male germ cell | 0 1 2 3 4 | female infertility (11557891), |
| | 21 0 | number (15774719), abnormal | | early reproductive |
| | | spermatogonia | | senescence (15774719)small |
| | | proliferation (15774719), | | ovary (11557891) abnormal |
| | | oligozoospermia (15774719), | | ovulation (11557891), increased |
| | | abnormal | | circulating follicle stimulating |
| | | spermatogenesis (15774719), | | hormone |
| | | abnormal acrosome | | level (15774719) abnormal |
| | | morphology (15774719), abnormal | | ovulation (11557891), absent |
| | | spermiogenesis (15774719) | | mature ovarian |
| | | | | follicles (11557891), impaired |
| | | | | ovarian |
| | | | | folliculogenesis (11557891) abnormal |
| | | | | oocyte |
| | | | | morphology (11557891) |
| Prss21 | 15 16 17 18 21 | abnormal | 4 5 | decreased fertilization |
| | | spermatogenesis (19571264), | | frequency (19571264), impaired |
| | | oligozoospermia (19571264), | | fertilization (18754795, 19571264) |
| | | teratozoospermia (19571264), | | decreased fertilization |
| | | abnormal sperm flagellum | | frequency (19571264), impaired |
| | | morphology (19571264), detached | | acrosome reaction (18754795), |
| | | sperm flagellum (19571264), | | impaired |
| | | abnormal sperm head | | fertilization (18754795, 19571264) |
| | | morphology (19571264), coiled | | |
| | | sperm flagellum (19571264), | | |
| | | hairpin sperm flagellum (19571264), | | |
| Tex19.1 | 15 16 17 1 10 20 | abnormal | 0 4 7 11 | reduced female |
| | 21 0 | spermatogenesis (18802469), | | fertility (18802469) abnormal |
| | | reduced male fertility (18802469), | | chiasmata |
| | | oligozoospermia (18802469), | | formation (21103378) abnormal |
| | | abnormal male meiosis (18802469), | | placenta morphology (23674551), |
| | | decreased male germ cell | | small |
| | | number (18802469), arrest of male | | placenta (23674551)decreased |
| | | meiosis (21103378), abnormal | | litter size (18802469, 23674551), |
| | | spermatogenesis (23674551), | | partial prenatal |
| | | abnormal | | lethality (18802469, 21103378) |
| | | spermiogenesis (23674551), male | | |
| | | infertility (23674551), reduced male | | |
| | | fertility (23674551), | | |
| | | oligozoospermia (23674551), | | |
| | | azoospermia (23674551), | | |
| | | teratozoospermia (23674551), arrest | | |
| | | of male meiosis (23674551), | | |
| Cul4a | 15 16 17 1 10 20 | abnormal | 0 4 5 8 11 | reduced female |
| | 21 0 | spermatogenesis (21291880), male | | fertility (21291880) abnormal cell |
| | | infertility (21291880), | | cycle checkpoint |
| | | oligozoospermia (21291880), | | function (19481525, 19430492), |
| | | azoospermia (21291880), abnormal | | abnormal DNA repair (19481525), |
| | | male meiosis (21291880), abnormal | | abnormal double-strand DNA |
| | | spermatocyte | | break repair (21291880), |
| | | morphology (21291880), abnormal | | chromosomal |
| | | spermatid morphology (21291880), | | instability (19430492) abnormal |
| | | abnormal sperm flagellum | | cell cycle checkpoint |
| | | morphology (21291880), abnormal | | function (19481525, 19430492), |
| | | sperm head morphology (21291880), | | abnormal double-strand DNA |
| | | | | break repair (21291880) abnormal |
| | | | | cell cycle checkpoint |
| | | | | function (19481525, 19430492)decreased |
| | | | | litter size (21291880) |
| Spef2 | 15 21 0 | male infertility (21715716), | None | |
| | | abnormal | | |
| | | spermatogenesis (21715716), | | |
| | | oligozoospermia (21715716), | | |
| | | abnormal sperm flagellum | | |
| | | morphology (21715716), short | | |
| | | sperm flagellum (21715716), | | |
| | | abnormal sperm axoneme | | |
| | | morphology (21715716), | | |
| Hspa4 | 15 16 17 1 10 20 | abnormal | None | |
| | 21 0 | spermatogenesis (21487003), male | | |
| | | infertility (21487003), | | |
| | | oligozoospermia (21487003), | | |
| | | abnormal spermatocyte | | |
| | | morphology (21487003), abnormal | | |
| | | spermatid morphology (21487003), | | |
| | | arrest of male meiosis (21487003), | | |
| Katnal1 | 15 10 21 0 | abnormal | None | |
| | | spermatogenesis (22654668), male | | |
| | | infertility (22654668), | | |
| | | oligozoospermia (22654668), | | |
| | | abnormal spermatid | | |
| | | morphology (22654668), | | |
| Ttll1 | 15 16 17 21 0 | short sperm flagellum (20498047), | None | |
| | | abnormal | | |
| | | spermatogenesis (20442420), male | | |
| | | infertility (20442420), | | |
| | | oligozoospermia (20442420), | | |
| | | teratozoospermia (20442420), | | |
| | | absent sperm flagellum (20442420), | | |
| | | detached sperm | | |
| | | flagellum (20442420), absent sperm | | |
| | | head (20442420), abnormal sperm | | |
| | | midpiece morphology (20442420), | | |
| Ppp1cc | 15 1 10 20 21 0 | abnormal spermiogenesis (9882500), | 5 | abnormal preimplantation embryo |
| | | male infertility (9882500), | | development (12606345) |
| | | oligozoospermia (9882500), | | |
| | | abnormal male meiosis (9882500), | | |
| | | decreased male germ cell | | |
| | | number (9882500), | | |
| | | globozoospermia (17301292), | | |
| | | oligozoospermia (12606345), | | |
| | | azoospermia (17301292), abnormal | | |
| | | spermatid morphology (17301292), | | |
| | | arrest of spermiogenesis (17301292), | | |
| | | abnormal sperm flagellum | | |
| | | morphology (17301292), abnormal | | |
| | | sperm head | | |
| | | morphology (17301292, 12606345), | | |
| | | pinhead sperm (17301292), | | |
| | | abnormal sperm midpiece | | |
| | | morphology (17301292), abnormal | | |
| | | sperm mitochondrial sheath | | |
| | | morphology (17301292), absent | | |
| | | sperm mitochondrial | | |
| | | sheath (17301292), abnormal sperm | | |
| | | principal piece | | |
| | | morphology (17301292), | | |
| | | multiflagellated sperm (12606345), | | |
| Lipe | 15 16 17 1 10 21 | male infertility (10639158), | 2 9 | decreased circulating leptin |
| | 0 9 | oligozoospermia (10639158), | | level (11316346, 18335062) abnormal |
| | | abnormal | | white adipose tissue |
| | | spermiogenesis (11564684), male | | physiology (10639158, 11717312), |
| | | infertility (11564684), | | decreased adiponectin |
| | | azoospermia (11564684), decreased | | level (18335062, 12865325), |
| | | male germ cell number (11564684), | | decreased subcutaneous adipose |
| | | abnormal spermatid | | tissue amount (18335062), |
| | | morphology (11564684), male | | increased brown adipose tissue |
| | | infertility (12835327), | | amount (11316346), increased fat |
| | | oligozoospermia (12835327), | | cell size (10639158), abnormal fat |
| | | | | cell morphology (11316346), |
| | | | | abnormal brown adipose tissue |
| | | | | morphology (10639158), |
| | | | | abnormal white fat cell |
| | | | | morphology (18335062), |
| | | | | decreased white adipose tissue |
| | | | | amount (18335062), abnormal |
| | | | | white adipose tissue |
| | | | | morphology (10639158), |
| | | | | abnormal brown adipose tissue |
| | | | | physiology (11717312), abnormal |
| | | | | abdominal fat pad |
| | | | | morphology (11316346) |
| Prnd | 15 16 17 18 21 0 | abnormal | 4 5 | impaired |
| | | spermiogenesis (12110578), male | | fertilization (15161660) impaired |
| | | infertility (12110578), | | acrosome |
| | | oligozoospermia (12110578), | | reaction (12110578, 15161660), |
| | | teratozoospermia (12110578), | | impaired fertilization (15161660) |
| | | abnormal acrosome | | |
| | | morphology (12110578), abnormal | | |
| | | sperm head morphology (12110578), | | |
| | | hairpin sperm flagellum (12110578), | | |
| | | male infertility (15161660), | | |
| Brwd1 | 15 16 17 18 1 10 | globozoospermia (18353305), | 0 4 5 | female |
| | 21 0 | abnormal sperm midpiece | | infertility (18353305) abnormal |
| | | morphology (18353305), decreased | | female meiosis (18353305), |
| | | male germ cell number (18353305), | | abnormal oocyte morphology ( ), |
| | | teratozoospermia (18353305), | | impaired |
| | | oligozoospermia ( ), abnormal male | | fertilization (18353305) impaired |
| | | germ cell morphology (18353305), | | fertilization (18353305) |
| | | male infertility (18353305), | | |
| | | abnormal spermiogenesis ( ), | | |
| | | abnormal sperm flagellum | | |
| | | morphology (18353305), | | |
| | | necrospermia (18353305), abnormal | | |
| | | sperm head morphology (18353305) | | |
| Cstf2t | 15 16 17 18 21 0 | abnormal | 4 5 | impaired |
| | | spermiogenesis (18077340), male | | fertilization (18077340) impaired |
| | | infertility (18077340), | | fertilization (18077340) |
| | | globozoospermia (18077340), | | |
| | | oligozoospermia (18077340), | | |
| | | teratozoospermia (18077340), | | |
| | | abnormal spermiation (18077340), | | |
| | | abnormal spermatid | | |
| | | morphology (18077340), | | |
| Rimbp3 | 15 18 19 20 21 0 | abnormal | 4 5 | impaired |
| | | spermiogenesis (19091768), male | | fertilization (19091768) impaired |
| | | infertility (19091768), | | fertilization (19091768) |
| | | oligozoospermia (19091768), | | |
| | | abnormal spermatid | | |
| | | morphology (19091768), detached | | |
| | | sperm flagellum (19091768), | | |
| | | abnormal sperm head | | |
| | | morphology (19091768), detached | | |
| | | acrosome (19091768), abnormal | | |
| | | sperm nucleus | | |
| | | morphology (19091768), kinked | | |
| | | sperm flagellum (19091768), | | |
| | | ectopic manchette (19091768), | | |
| Agtpbp1 | 15 16 17 1 10 21 0 | male infertility (1061118), | 0 11 | infertility ( ), reduced female |
| | | oligozoospermia (1061118), | | fertility (1061118,) decreased litter |
| | | abnormal male germ cell | | size (2726749) |
| | | morphology (1061118), male | | |
| | | infertility (11884758), | | |
| | | oligozoospermia (11884758), | | |
| | | abnormal male germ cell | | |
| | | morphology (11884758), male | | |
| | | infertility (2726749), | | |
| | | azoospermia (2726749), male | | |
| | | infertility (16465590), | | |
| | | azoospermia ( ), male infertility ( ), | | |
| | | abnormal male germ cell | | |
| | | morphology ( ), oligozoospermia ( ), | | |
| | | teratozoospermia ( ), reduced male | | |
| | | fertility (11884758), | | |
| Csnk2a2 | 15 16 17 1 19 20 | male infertility (10471512), | None | |
| | 21 0 | globozoospermia (10471512), | | |
| | | oligozoospermia (10471512), | | |
| | | teratozoospermia (10471512), | | |
| | | abnormal spermatid | | |
| | | morphology (10471512), abnormal | | |
| | | sperm head morphology (10471512), | | |
| | | detached acrosome (10471512), | | |
| | | abnormal sperm nucleus | | |
| | | morphology (10471512), kinked | | |
| | | sperm flagellum (10471512), | | |
| Cd59b | 15 16 17 1 10 21 0 | oligozoospermia (12594949, 16272280), | 0 11 | early reproductive |
| | | abnormal male germ cell | | senescence (12594949) decreased |
| | | morphology (12594949), abnormal | | litter size (12594949) |
| | | sperm head morphology (16272280), | | |
| | | absent sperm head (16272280), | | |
| | | necrospermia (16272280), | | |
| Pms2 | 15 1 10 20 21 0 | globozoospermia (7628019), | 4 5 13 14 | abnormal mismatch |
| | | decreased male germ cell | | repair (7628019, 20624957), |
| | | number (20624957), | | abnormal DNA repair (20624957), |
| | | teratozoospermia (20624957), | | chromosomal |
| | | oligozoospermia (7628019), | | instability (17785530) abnormal |
| | | abnormal male meiosis (7628019), | | mismatch |
| | | coiled sperm flagellum (20624957), | | repair (7628019, 20624957)* abnormal |
| | | male infertility (7628019, 20624957), | | class switch |
| | | short sperm flagellum (20624957), | | recombination (20624957) premature |
| | | abnormal sperm flagellum | | death (20624957, 20624957, 17785530, |
| | | morphology (7628019), abnormal | | 18264106) |
| | | sperm head morphology (20624957) | | |
| Cnot7 | 15 16 17 1 10 21 0 | male infertility (15107851), | 4 | abnormal |
| | | oligozoospermia (15107851), | | gametogenesis (15199137) |
| | | teratozoospermia (15107851), | | |
| | | abnormal male germ cell | | |
| | | morphology (15107851), abnormal | | |
| | | spermatid morphology (15107851), | | |
| | | abnormal sperm flagellum | | |
| | | morphology (15107851), abnormal | | |
| | | sperm head morphology (15107851), | | |
| | | abnormal sperm mitochondrial | | |
| | | sheath morphology (15107851), | | |
| | | male infertility (15199137), | | |
| | | oligozoospermia (15199137), | | |
| | | Sertoli cell hypoplasia (15199137), | | |
| | | decreased male germ cell | | |
| | | number (15199137), abnormal male | | |
| | | germ cell morphology (15199137), | | |
| Fhl5 | 15 16 17 10 19 20 | oligozoospermia (15247423), | 4 | abnormal |
| | 21 0 | teratozoospermia (15247423), | | gametogenesis (15247423) |
| | | abnormal acrosome | | |
| | | morphology (15247423), delayed | | |
| | | male fertility (15247423), abnormal | | |
| | | sperm head morphology (15247423), | | |
| | | detached acrosome (15247423), | | |
| | | abnormal sperm nucleus | | |
| | | morphology (15247423), hairpin | | |
| | | sperm flagellum (15247423), | | |
| Dnaja1 | 15 16 17 10 20 21 0 | reduced male fertility (15660130), | None | |
| | | oligozoospermia (15660130), | | |
| | | abnormal spermatocyte | | |
| | | morphology (15660130), abnormal | | |
| | | spermatid morphology (15660130), | | |
| Adad1 | 15 16 17 18 21 0 | male infertility (15649457), | 4 5 | impaired |
| | | oligozoospermia (15649457), | | fertilization (15649457) impaired |
| | | teratozoospermia (15649457), | | fertilization (15649457) |
| Creb3l4 | 15 1 10 21 | oligozoospermia (16107712), | None | |
| | | decreased male germ cell | | |
| | | number (16999736), abnormal | | |
| | | spermatid morphology (16999736), | | |
| Agfg1 | 15 16 17 18 10 19 | male infertility (11711676), | 4 5 | abnormal |
| | 20 21 0 | globozoospermia (11711676), | | gametogenesis (11711676), |
| | | oligozoospermia (11711676), | | impaired |
| | | teratozoospermia (11711676, 15705627), | | fertilization (11711676) impaired |
| | | arrest of | | fertilization (11711676) |
| | | spermiogenesis (11711676), absent | | |
| | | acrosome (11711676), abnormal | | |
| | | acrosome morphology (11711676), | | |
| | | abnormal sperm nucleus | | |
| | | morphology (11711676, 15705627), | | |
| | | enlarged sperm head (15705627), | | |
| | | absent sperm mitochondrial | | |
| | | sheath (11711676), multiflagellated | | |
| | | sperm (15705627), | | |
| Vps54 | 15 16 17 10 21 | globozoospermia (1955109), | 0 2 4 8 | infertility (7416238) decreased |
| | | oligozoospermia (1955109), | | circulating estrogen |
| | | | | level (210748) abnormal |
| | | | | gametogenesis (1955109)*embryonic |
| | | | | growth |
| | | | | retardation (16244655), complete |
| | | | | embryonic lethality during |
| | | | | organogenesis (16244655) |
| Gba2 | 15 18 1 10 21 0 | reduced male fertility (17080196), | 5 11 | abnormal |
| | | globozoospermia (17080196), | | fertilization (17080196) decreased |
| | | oligozoospermia (17080196), | | litter size (17080196) |
| | | abnormal male germ cell | | |
| | | morphology (17080196), | | |
| Rsph1 | 15 10 21 0 | male infertility (18453535), | None | |
| | | oligozoospermia (18453535), | | |
| | | abnormal spermatid | | |
| | | morphology (18453535), | | |
| Sgol2 | 15 10 20 21 0 | oligozoospermia (18765791), male | 0 4 | female |
| | | infertility (18765791), abnormal | | infertility (18765791) abnormal |
| | | spermatid morphology (18765791), | | gametogenesis (18765791), |
| | | arrest of male meiosis (18765791) | | abnormal female |
| | | | | meiosis (18765791) |
| Nphp1 | 15 16 1 10 21 0 | male infertility (18684731), | None | |
| | | oligozoospermia (18684731), | | |
| | | teratozoospermia (18684731), | | |
| | | abnormal spermiation (18684731), | | |
| | | decreased male germ cell | | |
| | | number (18684731), abnormal | | |
| | | sperm flagellum | | |
| | | morphology (18684731), | | |
| Fkbp4 | 15 16 17 18 1 10 | hairpin sperm flagellum (17307907), | 0 4 5 7 11 14 | female |
| | 21 2 0 | oligozoospermia (17307907), | | infertility (16176985, 16873445, 17142810) |
| | | increased circulating testosterone | | impaired |
| | | level (17142810), male | | fertilization (15831525, 16176985, |
| | | infertility (16176985, 15831525, 17307907), | | 17307907) impaired |
| | | reduced male | | fertilization (15831525, 16176985, |
| | | fertility (17142810) | | 17307907) abnormal uterine |
| | | | | environment (16176985), |
| | | | | abnormal |
| | | | | decidualization (16873445)decreased |
| | | | | litter size (17142810), |
| | | | | abnormal |
| | | | | superovulation (16873445), partial |
| | | | | prenatal lethality (15831525) |
| Herc2 | 15 1 21 0 | male infertility ( ), | 0 3 8 | female infertility ( ), reduced |
| | | oligozoospermia ( ), decreased male | | female fertility ( ) decreased |
| | | germ cell number ( ), abnormal | | corpora lutea number ( ) complete |
| | | spermatid morphology ( ), | | prenatal lethality ( ) |
| Usp1 | 15 1 10 20 21 0 | male infertility (19217432), | 0 4 | reduced female |
| | | oligozoospermia (19217432), | | fertility (19217432) induced |
| | | abnormal male germ cell | | chromosome |
| | | morphology (19217432), abnormal | | breakage (19217432), decreased |
| | | spermatogonia | | oocyte number (19217432) |
| | | morphology (19217432), abnormal | | |
| | | spermatocyte | | |
| | | morphology (19217432), abnormal | | |
| | | spermatid morphology (19217432), | | |
| Galnt3 | 15 10 21 0 | male infertility (19213845), | None | |
| | | oligozoospermia (19213845), | | |
| | | teratozoospermia (19213845), | | |
| | | oligozoospermia (22912827), | | |
| Gtsf1 | 15 1 10 20 21 0 | male infertility (19735653), | None | |
| | | oligozoospermia (19735653), | | |
| | | teratozoospermia (19735653), arrest | | |
| | | of male meiosis (19735653), | | |
| Aspm | 15 16 17 10 21 0 | reduced male fertility (20823249), | 0 1 | reduced female |
| | | oligozoospermia (20823249), small | | fertility (20823249) decreased |
| | | sperm head (20823249), | | ovary weight (20823249) |
| Ing2 | 15 16 17 1 10 20 | male infertility (21124965), | None | |
| | 21 0 | globozoospermia (21124965), | | |
| | | oligozoospermia (21124965), | | |
| | | teratozoospermia (21124965), | | |
| | | decreased male germ cell | | |
| | | number (21124965), arrest of male | | |
| | | meiosis (21124965), enlarged sperm | | |
| | | head (21124965), coiled sperm | | |
| | | flagellum (21124965), short sperm | | |
| | | flagellum (21124965), | | |
| | | multiflagellated sperm (21124965), | | |
| Zfp42 | 15 1 10 20 21 | oligozoospermia (21641340), | 4 5 8 11 | abnormal DNA |
| | | decreased male germ cell | | methylation (21233130), abnormal |
| | | number (21641340), abnormal | | imprinting (21233130) abnormal |
| | | sperm head morphology (21641340), | | DNA methylation (21233130), |
| | | kinked sperm flagellum (21641340), | | abnormal |
| | | | | imprinting (21233130) abnormal |
| | | | | imprinting (21233130)decreased |
| | | | | litter size (21233130), partial |
| | | | | embryonic lethality (21233130), |
| | | | | partial prenatal |
| | | | | lethality (21233130) |
| Spink2 | 15 1 10 21 0 | reduced male fertility (21705336), | 11 | decreased litter |
| | | oligozoospermia (21705336), | | size (21705336)reduced male |
| | | teratozoospermia (21705336), | | fertility (21705336), |
| | | kinked sperm flagellum (21705336), | | oligozoospermia (21705336), |
| | | | | teratozoospermia (21705336), |
| | | | | kinked sperm |
| | | | | flagellum (21705336), |
| Mcm9 | 15 1 10 20 21 | oligozoospermia (21987787), | 0 1 2 3 4 | female |
| | | decreased male germ cell | | infertility (21987787, 22771120)ovary |
| | | number (21987787), abnormal | | hyperplasia (21987787), |
| | | spermatogonia | | decreased primordial germ cell |
| | | morphology (21987787), arrest of | | number (21987787) abnormal |
| | | male meiosis (21987787), | | ovary |
| | | oligozoospermia (22771120), | | physiology (21987787) abnormal |
| | | | | ovary physiology (21987787), |
| | | | | decreased primordial ovarian |
| | | | | follicle number (21987787), |
| | | | | abnormal ovarian follicle |
| | | | | number (21987787)spontaneous |
| | | | | chromosome |
| | | | | breakage (21987787, 22771120), |
| | | | | decreased oocyte |
| | | | | number (21987787), abnormal |
| | | | | female germ cell |
| | | | | morphology (21987787) |
| Catsperd | 15 16 17 10 21 0 | male infertility (21224844), | 1 | abnormal germ cell |
| | | oligozoospermia (21224844), | | morphology (21224844) |
| | | teratozoospermia (21224844), | | |
| Odf1 | 15 16 17 18 21 0 | male infertility (22037768), | 5 | impaired acrosome |
| | | oligozoospermia (22037768), | | reaction (22037768) |
| | | detached sperm | | |
| | | flagellum (22037768), coiled sperm | | |
| | | flagellum (22037768), abnormal | | |
| | | sperm midpiece | | |
| | | morphology (22037768), abnormal | | |
| | | sperm mitochondrial sheath | | |
| | | morphology (22037768), | | |
| Mns1 | 15 16 17 10 21 0 | male infertility (22396656), | None | |
| | | oligozoospermia (22396656), | | |
| | | abnormal spermatid | | |
| | | morphology (22396656), abnormal | | |
| | | sperm flagellum | | |
| | | morphology (22396656), kinked | | |
| | | sperm flagellum (22396656), short | | |
| | | sperm flagellum (22396656), | | |
| | | abnormal sperm mitochondrial | | |
| | | sheath morphology (22396656), | | |
| | | abnormal sperm principal piece | | |
| | | morphology (22396656), abnormal | | |
| | | sperm axoneme | | |
| | | morphology (22396656), | | |
| Katnb1 | 15 16 17 10 20 21 0 | male infertility (22654669), | 4 | abnormal meiotic spindle |
| | | globozoospermia (22654669), | | morphology (22654669) |
| | | oligozoospermia (22654669), | | |
| | | abnormal male meiosis (22654669), | | |
| | | abnormal spermatid | | |
| | | morphology (22654669), abnormal | | |
| | | sperm flagellum | | |
| | | morphology (22654669), abnormal | | |
| | | manchette morphology (22654669), | | |
| | | abnormal sperm axoneme | | |
| | | morphology (22654669), increased | | |
| | | Sertoli cell | | |
| | | phagocytosis (22654669), | | |
| Rabl2 | 15 16 17 10 21 0 | male infertility (23055941), | None | |
| | | oligozoospermia (23055941), short | | |
| | | sperm flagellum (23055941), | | |
| Alkbh5 | 15 1 10 21 0 | reduced male fertility (23177736), | 11 | decreased litter size (23177736) |
| | | oligozoospermia (23177736), | | |
| | | teratozoospermia (23177736), | | |
| M1ap | 15 1 10 20 21 0 | male infertility (23269666), | 4 5 | abnormal double-strand DNA |
| | | globozoospermia (23269666), | | break repair (23269666) abnormal |
| | | oligozoospermia (23269666), | | double-strand DNA break |
| | | abnormal male meiosis (23269666), | | repair (23269666) |
| | | abnormal male germ cell | | |
| | | morphology (23269666), arrest of | | |
| | | male meiosis (23269666), abnormal | | |
| | | X-Y chromosome synapsis during | | |
| | | male meiosis (23269666), | | |
| Eno4 | 15 16 17 10 21 0 | male infertility (23446454), | None | |
| | | oligozoospermia (23446454), | | |
| | | abnormal sperm flagellum | | |
| | | morphology (23446454), kinked | | |
| | | sperm flagellum (23446454), | | |
| | | abnormal sperm midpiece | | |
| | | morphology (23446454), abnormal | | |
| | | sperm annulus | | |
| | | morphology (23446454), absent | | |
| | | sperm annulus (23446454), | | |
| | | abnormal sperm principal piece | | |
| | | morphology (23446454), abnormal | | |
| | | sperm axoneme | | |
| | | morphology (23446454), | | |
| Jmjd1c | 15 1 10 21 0 | oligozoospermia (24006281), | 0 | early reproductive |
| | | decreased male germ cell | | senescence (24006281) |
| | | number (24006281), abnormal | | |
| | | spermatogonia | | |
| | | morphology (24006281), | | |
| Atat1 | 15 16 17 1 10 21 0 | reduced male fertility (23748901), | 11 | decreased litter size (23748901) |
| | | oligozoospermia (23748901), | | |
| | | teratozoospermia (23748901), short | | |
| | | sperm flagellum (23748901), | | |
| | | abnormal sperm annulus | | |
| | | morphology (23748901), | | |
| Inft4 | 15 16 17 21 0 | oligozoospermia (14711786), | None | |
| | | arrest of spermatogenesis | | |
| | | (14711786, 12955145) | | |
| Inft8 | 15 16 17 21 0 | oligozoospermia (14711786), | None | |
| | | arrest of spermatogenesis | | |
| | | (12955145, 14711786) | | |
| Inft9 | 15 16 17 21 0 | arrest of spermatogenesis | None | |
| | | (14711786, 12955145), | | |
| | | oligozoospermia (14711786) | | |
| Esgd12d | 15 16 17 21 0 | teratozoospermia ( ), | None | |
| | | oligozoospermia ( ), | | |
| | | abnormal spermatogenesis | | |
| | | (12855593), | | |
| | | azoospermia (12855593) | | |
| Swm2 | 15 16 17 18 19 20 | abnormal spermatogenesis | 5 | impaired fertilization (16920728) |
| | 21 0 | (12855593), | | |
| | | teratozoospermia (16920728), | | |
| | | abnormal sperm nucleus | | |
| | | morphology (16920728), | | |
| | | oligozoospermia | | |
| | | (12855593, 16920728) | | |
| Repro13 | 15 21 0 | oligozoospermia ( ), abnormal | None | |
| | | spermatogenesis ( ) | | |
| Repro54 | 15 16 17 10 21 0 | teratozoospermia ( ), abnormal | None | |
| | | spermiogenesis ( ), oligozoospermia ( ) | | |
| Repro10 | 15 16 17 21 0 | oligozoospermia ( ) | None | |
| Repro16 | 15 16 17 10 21 0 | oligozoospermia ( ) | None | |
| Repro17 | 15 16 17 21 0 | oligozoospermia ( ) | None | |
| Repro20 | 15 16 17 10 21 0 | oligozoospermia ( ) | None | |
| Repro21 | 15 16 17 10 21 0 | oligozoospermia ( ) | None | |
| Repro24 | 15 16 17 21 0 | oligozoospermia ( ) | None | |
| Repro26 | 15 16 17 10 21 0 | oligozoospermia ( ) | None | |
| Repro19 | 15 16 17 10 21 2 0 | teratozoospermia ( ), | 2 | increased circulating luteinizing |
| | | oligozoospermia ( ) | | hormone level ( ) |
| Repro2 | 15 16 17 18 19 20 | abnormal sperm nucleus | 5 | impaired fertilization (16920728) |
| | 21 0 | morphology (16920728), | | |
| | | teratozoospermia (16920728), | | |
| | | oligozoospermia (16920728) | | |
| Repro3 | 15 16 17 18 19 20 | oligozoospermia (16920728), | 5 | impaired fertilization (16920728) |
| | 21 0 | abnormal sperm nucleus | | |
| | | morphology (16920728), | | |
| | | teratozoospermia (16920728) | | |
| Rnf8 | 15 10 20 21 0 | abnormal | 4 11 | induced chromosome |
| | | spermatogenesis (20385750), | | breakage (20385750), spontaneous |
| | | azoospermia (20385750), | | chromosome |
| | | oligozoospermia (20385750) | | breakage (20385750), decreased |
| | | | | litter size (20385750) |
| Tssk1 | 15 16 17 21 0 | oligozoospermia (20053632), | None | |
| | | teratozoospermia (20053632), | | |
| | | abnormal | | |
| | | spermatogenesis (20053632) | | |
| Bglap | 15 1 10 20 21 2 9 | oligozoospermia (21333348) | 2 9 | increased circulating estradiol |
| | | | | level (21333348), increased |
| | | | | circulating luteinizing hormone |
| | | | | level (21333348), increased total |
| | | | | body fat amount (17693256), |
| | | | | abnormal fat cell |
| | | | | morphology (17693256) |

Using this approach, we identified 6,030 genetic loci in association with fertility-related phenotypes in male and female mice, including genes with multiple family members, and quantitative trait loci.

By identifying the human orthologs (and subsequently paralogs) of these loci, we are able to predict how they function in human reproduction, fecundity, and fertility. Many of these genes have never been associated with mechanisms of human reproduction or infertility, thus we provide novel gene targets.

Many human genes have more than one mouse ortholog and the phenotypes associated with these different orthologs may be different, perhaps reflecting mechanisms of genetic divergence and the possibility that the phenotypes associated with variants in particular human loci may be more severe or wide-ranging than those associated with their orthologs in other species.

Our algorithmic approach led to the inclusion of 1,056 genetic loci in our knowledgebase whose associated phenotypes linked them to at least one aspect of male-specific reproductive biology. Here, we present specific examples that highlight how our dataset (Table 6) can be used to expand our understanding of the genetics of male fertility, and identify candidates for study in humans as biomarkers of infertility.

The phenotypes of 254 genes demonstrated that they function in spermiogenesis, the process involving the morphological differentiation of spermatozoa during spermatogenesis. 100 of these genes were associated with sperm count phenotypes. For example, 80 of these genes were also reported to result in ‘oligozoospermia’ phenotypes, thus each of these 80 genes is assigned (at least) both ‘15’ and ‘21’ male-specific phenotypic categories. This suggests that, for at least some of these 80 genes, the role they play in spermiogenesis (upstream of spermatogenesis) is the contributing factor to an oligospermia phenotype. As indicated in Table 6, alteration to the Cyp19a1 gene is associated with ‘abnormal spermiogenesis’ and ‘oligozoospermia’. CYP19A1 is expressed in both mouse and human sperm, and variants in CYP19A1 have been associated with aromatase-deficiency phenotypes in men, including infertility. This example confirms a route between a biological process (spermiogenesis), a clinical parameter (sperm count) and a genetic variant in a set of 100 genes that become candidate biomarkers of human male infertility. Interestingly, our dataset shows that 8 of these genes, including for example Cyp19a1, are associated with endocrine dysfunction. Mutations in CYP19A1 have been associated with aromatase-deficiency phenotypes in men, including infertility. The remaining genes in this subgroup could therefore represent candidate markers of patients with oligozoospermia that may respond to hormonal-based therapies.

Besides making [sometimes non-obvious] links between male phenotypes that may explain the etiology of male infertility, our dataset allows us to assess phenotypes comparatively between female and males. Depending on the genes and phenotypes involved, this could have a number of different implications for human fertility.

A number of genes in Table 6 are associated with spermiogenesis phenotypes in males as well as oogenesis and/or ‘oocyte-to-embryo transition’ phenotypes in females. Both paternal and maternal physiology and genetics contribute to the fecundity of mating pairs in most mammalian species. Therefore, these genes represent candidates that, if mutated in both a male partner and a female partner, contribute to reproductive complications, such as longer times to live birth (with and without the use of ARTs), or complicated pregnancies. Examples of these genes include Camk4, Sirt1, Brwd1, Agtpbp1, Agfg1, Vps54, Sgol2, Fkbp4, Herc2, Usp1, and Zfp42. A number of the 80 genes are specific to the process of meiosis (e.g., Meig1 and Katnb1). Since both male and female gametogenesis involves meiosis, one expects the phenotypes to be reported for both male and female mice when these genes are targeted. However, in the case of Meig1 there are no reports of any reproductive phenotype in females. While this could indicate that there is no female-specific phenotype for Meig1, it could also indicate that female Meig1-targeted mice have not been carefully studied, thus Meig1 becomes a candidate for study in female gametogenesis.

Some of the genes in Table 6, such as H1fnt, are reported to be specific to the testis in their expression and function. Indeed, male H1fnt mice have reduced fertility due to spermiogenesis defects and, as a result, impaired fertilization (Martianov et al., 2005; Tanaka et al., 2005). Thus, H1fnt mouse lines are difficult to maintain. Interestingly, this phenotype is rescued with the use of intracytoplasmic sperm injection (ICSI) as a means of fertilization, but not IVF (Tanaka et al., 2005). H1fnt is one of a number of genes that fit a similar fertility-related paradigm, namely Prss21, Tex19.1, Prnd, Cstf2t, Rimbp3, Adad1, Fkbp4, and Odf1. Variants that alter the function or expression of these genes could therefore represent excellent candidates to study in humans, in order to establish whether these genes, genetic variants within the genes (or functionally related genes), might identify couples for whom ICSI, rather than IVF, is likely to be a more efficient route to conception.

In this study we comprehensively classified genetic loci according to their role in mechanisms of reproduction and male and female fertility, which has clarified how particular genes and genetic variants functionally contribute to the pathophysiology of infertility disorders. By highlighting genetic loci for which relatively little existing information links them mechanistically to human infertility, we provide novel, clinically actionable molecular targets.

What is claimed is:

1. A method of generating a combined fertility potential profile of a female and a male, comprising:
obtaining input data representative of one or more fertility-associated genomic, phenotypic, and/or environmental exposure characteristics from a female and a male;
obtaining reference data representative of one or more fertility-associated genomic, phenotypic, and environmental characteristics from a reference set of females and a reference set of males;
using a computer system comprising a processor coupled to a memory for:
training the reference data by determining one or more correlations between the reference data and known pregnancy and infertility-related outcomes from the reference set of females and the reference set of males to provide determinants of fertility;
applying the determinants to the input data to generate a combined fertility potential profile of the male and female.

2. The method of claim 1, wherein the one or more fertility-associated genetic characteristic is a genetic variant.

3. The method of claim 1, wherein the one or more fertility-associated genetic characteristic is a gene product of a gene having a genetic variant.

4. The method of claim 1, wherein the infertility-associated phenotypic and/or environmental characteristic is selected from Table 3.

5. The method of claim 1, wherein the infertility-associated phenotypic and/or environmental characteristic is obtained from at least one selected from the group consisting of a questionnaire, a medical history, a family medical history, results of an assay run on a sample from a person, and combinations thereof.

6. The method of claim 4, wherein the person is selected from the group consisting of: the female, the male, the intimate partner of the female or male, blood-related relatives of the female or male, and combinations thereof.

7. The method of claim 1, wherein the input data is obtained from conducting an assay on a sample from the male and the female.

8. The method of claim 7, wherein said sample is a human tissue or bodily fluid.

9. The method of claim 7, wherein the assay comprises determining the presence of at least one variant in one or more genes.

10. The method of claim 9, wherein the variant is selected from the group consisting of: a single nucleotide polymorphism, a deletion, an insertion, a rearrangement, a copy number variation, and a combination thereof.

11. The method of claim 9, wherein the assay is selected from the group consisting of: sequencing, hybridization to an array, and amplification.

12. The method of claim 7, wherein the assay comprises determining levels of one or more gene products.

13. A method of generating a combined fertility profile of a female and a male, comprising:
obtaining input data representative of one or more fertility-associated genomic, phenotypic, and/or environmental exposure characteristics from a female and a male;
obtaining reference data representative of one or more fertility-associated genomic, phenotypic, and environmental characteristics from a reference sets of females and a reference set of males;
using a computer system comprising a processor coupled to a memory for:
identifying variables predictive of infertility from the reference data;
generating weighted predictor variables based on a magnitude of change in fertility attributed to each predictor variable;
applying the weighted predictor variables to the to the input data to generate a fertility profile that reflects the combined fertility profile of the male and the female.

13. The method of claim 12, wherein the fertility-associated genetic characteristic is a genetic variant.

14. The method of claim 12, wherein the fertility-associated genetic characteristic is a gene product of a gene having a genetic variant.

15. The method of claim 12, wherein at least one infertility-associated phenotypic and/or environmental characteristic is selected from Table 3.

16. The method of claim 12, wherein the genotypic, phenotypic and/or environmental characteristics are obtained from the male and the female are obtained from at least one selected from the group consisting of a questionnaire, a medical history, a family medical history, results of an assay run on a sample from a person, and combinations thereof.

17. The method of claim 12, wherein the input data is obtained from conducting an assay on a sample from the male and the female.

18. The method of claim 17, wherein the assay comprises determining the presence of at least one variant in one or more genes.

19. The method of claim 18, wherein the variant is selected from the group consisting of: a single nucleotide polymorphism, a deletion, an insertion, a rearrangement, a copy number variation, and a combination thereof.

20. The method of claim 18, wherein the assay is selected from the group consisting of: sequencing, hybridization to an array, and amplification.

21. The method of claim 17, wherein the assay comprises determining levels of one or more gene products.

22. A system for determining the combined fertility potential of a female and a male, the system comprising:
a processor; and
a computer-readable storage device containing instructions that when executed by the processor cause the system to:
accept as input data, data representative of one or more fertility-associated genomic, phenotypic, and/or environmental exposure characteristics from a female and a male;
analyzing the input data using a predictor generated by:
obtaining a reference set of fertility-associated genomic, phenotypic, and/or environmental exposure characteristics data from a plurality of men and women;
training the predictor with said reference set data to provide outputs indicative of the combined fertility potential of a female and a male;
running an algorithm on said input data, the algorithm having been trained on a reference set of data obtained from a plurality of men and women to provide a probability of achieving a pregnancy at a selected point in time as a result of using the prognosis predictor on the input data; and
generating a fertility profile as a result of running an algorithm on said input data, the algorithm having been trained on a reference set of data obtained from a plurality of men and women.

23. A method for assessing the infertility and/or fertility of a male subject comprising:
conducting an analysis of genetic and phenotypic traits of the male subject, the analysis comprising:
conducting a laboratory procedure on a sample obtained from the male subject to determine the presence of one or more genetic biomarkers;
obtaining one or more phenotypic traits and/or environmental exposures of the male subject; and
assessing fertility and/or infertility of the male subject by applying weighted predictor variables identified from genetic, phenotypic and environmental exposure data obtained from a reference population to the results of the analysis.
